(19) United States
(12) Patent Application Publication
(10) Pub. No.: US 2009/0275031 A1
(43) Pub. Date: Nov. 5, 2009

(54) Biomolecular nano device

(75) Inventors: Maria E. Tanner, Wake Forest, NC (US); Elizabeth A. Vasievich, Chapel Hill, NC (US); Jonathan M. Protz, Durham, NC (US); Thomas H. Labean, Hillsborough, NC (US)

Correspondence Address: JENKINS, WILSON, TAYLOR & HUNT, P. A., Suite 1200 UNIVERSITY TOWER, 3100 TOWER BLVD.,, DURHAM, NC, 27707, US

(73) Assignee: Duke University, Durham, NC (US)

(21) Appl. No.: 12/383,996

(22) Filed: Mar. 31, 2009

Related U.S. Application Data
(60) Provisional application No. 61/072,439, filed on Mar. 31, 2008.

(51) Int. Cl. C12Q 1/68; C12Q 1/00; C12Q 1/48; C12P 19/34; C12N 15/74; C12N 1/20; C12N 1/21; G01N 33/68; G01N 33/50; C07K 2/00; C07H 21/00; C12N 9/00

(52) U.S. Cl. 435/6; 435/4; 435/15; 435/91.5; 435/91.52; 435/471; 435/252.1; 435/252.3; 436/86; 436/94; 530/300; 536/25.3; 435/183

(57) ABSTRACT

Methods for measuring environmental parameters using chemical recording are provided. In some embodiments, the methods include generating a polymer comprising an ordered series of chemical units, wherein the position and number of each chemical unit in the polymer is indicative of a reading of the environmental state variable at a given point in time. The presently disclosed subject matter also provides compositions that can be employed in and/or that employ the disclosed methods for recording environmental state variables.

### RELATED APPLICATIONS

This application claims priority to U.S. Provisional Patent Application Ser. No. 61/072,439, filed Mar. 31, 2008, which is herein incorporated in its entirety.

### TECHNICAL FIELD

The presently disclosed subject matter relates to methods for chemical recording of environmental state variables. The presently disclosed subject matter also relates to compositions that employ the disclosed methods for recording environmental state variables.

### BACKGROUND

Nanosensors have tremendous potential utility in most commercial industries, including medicine, manufacturing, consumer products, and defense. Size, weight, and power consumption are critical properties to most systems. Reductions in these properties allow for more capable systems, often at reduced costs, especially in terms of cheaper system integration. Nanosensors offer similar or superior performance as conventional macro- or micro-sensors but are packaged in a fraction of the size and use significantly less power. This savings in size allows for smaller and lighter end products.

With smaller size, lower cost, and higher sensitivity, nanosensors will be incorporated in many commercial applications including medicine, manufacturing, and consumer goods. In medicine, nanosensors will be used to provide cost-effective diagnostic tools and enhanced drug delivery. Further in the future, they might be used to monitor and repair cell damage. Nanosensors could be used in manufacturing to provide greater and more reliable quality control. Consumers will use nanosensors in devices that are significantly smaller and more powerful such as cell phones, computers, and GPS units. Cars and other sources of transportation will incorporate nanosensors for enhanced safety.

Another area where nanosensors can and will be incorporated is national security and defense technologies. They will be used in chemical and biological sensing and to increase soldier survivability through lighter and more effective protection, superior communication and surveillance capabilities, and health monitoring. Nanosensors could be built into skin-integral survivability countermeasures, which decrease the demand for sensors and countermeasures to be placed at the top of a ground vehicle and allow for the graceful degradation of the sensors because of the redundancy that can be built into the system. They can be used for tracking and to produce more accurate guided munitions and faster, lighter, and better protected ground vehicles. Just considering military applications alone, millions of nanosensors could be integrated into existing and future systems. Nanosensors will have a lasting impact in most, if not all, commercial and defense applications.

An example of a conventional sensor that could be scaled down to a nanosensor is an inertial measurement unit (IMU). An IMU is a system used to detect and record position similar to a global positioning system (GPS). However, GPS uses an external satellite to detect position, while an IMU is a closed system comprised of three accelerometers and three gyroscopes. This arrangement provides data on linear acceleration in three orthogonal directions and also rotational data. A nano-IMU has several potential applications, including targeted drug delivery and tracking. Targeted drug delivery could be achieved by using the nano-IMU to direct the drug to a targeted and specific area in the body. Another option is to use the nano-IMU for tracking where an object has been, by changing the frame of reference. Or it could simply be used as a replacement for larger IMUs to make end products smaller, lighter, more powerful, and cheaper.

Currently, there is a wide range of accelerometers and mechanisms through which they can sense acceleration. Piezoelectric accelerometers generate a voltage corresponding to the acceleration; capacitive accelerometers use the change in capacitance, which is converted to a voltage; piezoresistive accelerometers measure acceleration through the change in resistance. These devices and other varieties of accelerometers not described generally use electronics at some point to sense the acceleration or to transmit an output. However, at a nano scale, electronics are exceedingly difficult, if not impossible, to fabricate. Molecular transistors have recently been developed in an effort to fabricate smaller electronics. One version consists of a carbon nanotube connecting a source electrode at one end and a drain electrode at the other end (Tans et al., 1998; Bachtold et al., 2001). Here as well, however, it is exceedingly difficult to achieve acceptable electrical contact; the electrodes are patterned on the wafer and then nanotubes are placed until one connects a pair of electrodes.

What are needed, then, are new methods for recording environmental state variables that can be performed on the nano scale, and compositions that employ such methods.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides methods for recording of an environmental state variable. In some embodiments, the methods comprise generating a polymer comprising an ordered series of chemical units, wherein the position and number of each chemical unit in the polymer is indicative of a reading of the environmental state variable at a given point in time. In some embodiments, the environmental state variable is selected from the group consisting of position, velocity, acceleration, temperature, pressure, fluorescence, concentration, and pH, intensity of sound, intensity of light or electromagnetic radiation, and strength of magnetic field. In some embodiments, the chemical units are selected from the group consisting of sugars, amino acids, and nucleotides. In some embodiments, the chemical units are nucleotides. In some embodiments, the methods further comprise determining the nucleotide sequence of the polymer.

In some embodiments, the chemical units are present within a plurality of reservoirs, and further wherein (a) each member of the plurality of reservoirs is designed to release one or more of the chemical units present therein when the reservoir experiences an environmental state variable that exceeds a minimum threshold; and (b) each of the one or more chemical units that are released enters a reaction chamber in which the polymer is generated. In some embodiments, each of the plurality of reservoirs comprises a thermosensitive liposome designed to release one or more chemical units contained therein if the thermosensitive liposome experiences a temperature exceeding a minimum temperature. In some embodiments, the plurality of reservoirs comprises at least two different classes of thermosensitive liposomes, each class of thermosensitive liposomes having a different threshold above which the thermosensitive liposome releases one or more chemical units contained therein. In some embodiments, the chemical units present within the thermosensitive liposomes are identical among members of the same class of thermosensitive liposomes but are different among different classes of thermosensitive liposomes. In some embodiments, each chemical unit comprises a double stranded region and a single stranded overhang, and further wherein the chemical units present within different classes of thermosensitive liposomes differ in the sequence of the double stranded region but not the sequence of the single stranded overhang. In some embodiments, each chemical unit comprises a single nucleotide, and further wherein the chemical units present within different classes of thermosensitive liposomes differ in the particular type of nucleotide.

In some embodiments, each chemical unit comprises a single DNA strand, and further wherein the chemical units present within different classes of thermosensitive liposomes differ in the sequence of the single DNA strand. In some embodiments, the method further comprises a DNA strand complement. In some embodiments, the method further comprises a start sequence. In some embodiments, the reaction chamber comprises an enzyme that polymerizes the chemical units present therein to form the polymer. In some embodiments, the enzyme is selected from the group consisting of a ligase and a terminal deoxynucleotidyl transferase. In some embodiments, the enzyme is a ligase and the reaction chamber further comprises all reagents necessary to produce the polymer. In some embodiments, each of the plurality of reservoirs comprises a photosensitive liposome designed to release one or more chemical units contained therein if the photosensitive liposome experiences an incident light intensity exceeding a minimum threshold. In some embodiments, the plurality of reservoirs comprises at least two different classes of photosensitive liposomes, each class of photosensitive liposomes having a different threshold above which the photosensitive liposome releases one or more chemical units contained therein. In some embodiments, the chemical units present within the photosensitive liposomes are identical among members of the same class of photosensitive liposomes but are different among different classes of photosensitive liposomes. In some embodiments, each chemical unit comprises a double stranded region and a single stranded overhang, and further wherein the chemical units present within different classes of photosensitive liposomes differ in the sequence of the double stranded region but not the sequence of the single stranded overhang.

In some embodiments, each of the plurality of reservoirs comprises a protein or protein complex designed to release one or more chemical units contained therein by undergoing a conformational change in response to a stimulus such as a change in the temperature, pressure, salinity, or pH, of the environment which surround the protein or protein complex or to a binding event with an antigen or other molecule or particle. In some embodiments, the plurality of reservoirs comprises at least two different classes of protein or protein complex, each class of which is responsive to a different stimulus. In some embodiments, the chemical units present within the proteins or protein complexes are identical among members of the same class of proteins or protein complexes but are different among different classes of proteins or protein complexes. In some embodiments, each chemical unit comprises a double stranded region and a single stranded overhang, and further wherein the chemical units present within different classes of proteins or protein complexes differ in the sequence of the double stranded region but not the sequence of the single stranded overhang. In some embodiments, each of the plurality of reservoirs comprises a porous or perforated shell designed to release one or more chemical units contained therein by undergoing a change in porosity in response to a stimulus such as a change in the temperature, pressure, salinity, or pH, of the environment which surround the protein or protein complex or to a binding event with an antigen or other molecule or particle. In some embodiments, the porous or perforated shell is made from a plastic or organic polymer. In some embodiments, the plurality of reservoirs comprises at least two different classes of porous or perforated shell, each class of which is responsive to a different stimulus. In some embodiments, the chemical units present within the porous or perforated shells are identical among members of the same class of proteins or protein complexes but are different among different classes of porous or perforated shells. In some embodiments, each chemical unit comprises a double stranded region and a single stranded overhang, and further wherein the chemical units present within different classes of porous or perforated shells differ in the sequence of the double stranded region but not the sequence of the single stranded overhang. In some embodiments, each chemical unit comprises a single DNA strand, and further wherein the chemical units present within different classes of thermosensitive liposomes differ in the sequence of the single DNA strand. In some embodiments, the method further comprises a DNA strand complement. In some embodiments, the method further comprises a start sequence. In some embodiments, the porous or perforated shell is made from a ceramic. In some embodiments, the porous or perforated shell is made from a metal.

In some embodiments, the reaction chamber is a liposome. In some embodiments, the reaction chamber is a liposome contained within a vesosome. In some embodiments, the reaction chamber is a vesosome. In some embodiments, sensed information is simultaneously reported it is being chemically recorded. In some embodiments, the reporting signal is a level of fluorescence. In some embodiments, a particular fluorophore is associated with DNA binding so that the detectable fluorescence is altered as units are bound and/or to particular sequences so that the detectable fluorescence is altered in the mixing chamber as more chemical units are added.

In some embodiments, the chemical units are selected from the group of small particles, which might or might not be chemically inert. In some embodiments, the small particles are selected from the group consisting of metal beads, plastic beads, ferromagnetic beads, electrostatically-charged dielectric beads.

In some embodiments, the chemical units are selected from the group consisting of bacteria, archaea, and eukaryotic cells. In some embodiments, the chemical units are bound to a plurality of surfaces, wherein (a) each member of the plurality of surfaces is designed to release one or more of the chemical units present thereon when the surface experiences an environmental state variable that exceeds a minimum threshold; and (b) each of the one or more chemical units that are released enters a reaction chamber in which the polymer is generated.

In some embodiments, the chemical units are initially possessed of one of a plurality of specific conformations, each of which makes the chemical units unavailable for incorporation into a polymer, wherein (a) one or more of the chemical units which possess a conformation corresponding to each member of the plurality of conformations is designed to take on a new conformation when the unit experiences a change in an environmental state variable that exceeds a minimum threshold; and (b) each of the one or more chemical units that undergoes a change in conformation adopts a new conformation that makes it available incorporation into the polymer that is being generated in the reaction chamber.

The presently disclosed subject matter also provides compositions for chemical recording. In some embodiments, the compositions comprise (a) a plurality of reservoirs each containing one or more chemical units, wherein each reservoir is characterized by a thermal stability point at or above which one or more of the chemical units present within the reservoir is released from the reservoir; (b) a reaction chamber in which the one or more chemical units that have been released collect; and (c) an enzyme and all other reagents necessary for polymerizing the one or more chemical units present in the reaction chamber to form a polymer. In some embodiments, the plurality of reservoirs comprises one or more different classes of reservoirs, each class of reservoir having a different threshold above which the reservoir releases one or more chemical units contained therein. In some embodiments, the plurality of reservoirs comprises at least two different classes of thermosensitive liposomes, and further wherein each class of thermosensitive liposomes is characterized by a different threshold above which the thermosensitive liposome releases one or more of the chemical units contained therein. In some embodiments, the chemical units present within the thermosensitive liposomes are identical among members of the same class of thermosensitive liposomes but are different among different classes of thermosensitive liposomes. In some embodiments, each chemical unit comprises a nucleic acid molecule comprising a double stranded region and a single stranded overhang, and further wherein the chemical units present within different classes of thermosensitive liposomes differ in the sequence of the double stranded region but not the sequence of the single stranded overhang. In some embodiments, each chemical unit comprises a single DNA strand, and further wherein the chemical units present within different classes of thermosensitive liposomes differ in the sequence of the single DNA strand. In some embodiments, the composition further comprises a DNA strand complement. In some embodiments, the composition further comprises a start sequence. In some embodiments, the reaction chamber comprises an enzyme that polymerizes the chemical units present therein to form the polymer. In some embodiments, the enzyme is selected from the group consisting of a ligase and a terminal deoxynucleotidyl transferase. In some embodiments, the enzyme is a ligase and the reaction chamber further comprises all reagents necessary to produce the polymer.

In some embodiments, the compositions for chemical recording comprise (a) a plurality of photosensitive liposomes each containing one or more chemical units, wherein each photosensitive liposome is characterized by a stability point governed by the intensity of light or electromagnetic radiation incident on the reservoir such that beyond a given threshold for said light intensity one or more of the chemical units present within the reservoir is released from the reservoir; (b) a reaction chamber in which the one or more chemical units that have been released collect; and (c) an enzyme and all other reagents necessary for polymerizing the one or more chemical units present in the reaction chamber to form a polymer. In some embodiments, the plurality of reservoirs comprises one or more different classes of photosensitive liposomes, each class of reservoir having a different threshold above which the reservoir releases one or more chemical units contained therein. In some embodiments, the plurality of reservoirs comprises at least two different classes of photosensitive liposomes, and further wherein each class of photosensitive liposomes is characterized by a different threshold above which the photosensitive liposome releases one or more of the chemical units contained therein. In some embodiments, the chemical units present within the photosensitive liposomes are identical among members of the same class of photosensitive liposomes but are different among different classes of photosensitive liposomes. In some embodiments, each chemical unit comprises a nucleic acid molecule comprising a double stranded region and a single stranded overhang, and further wherein the chemical units present within different classes of photosensitive liposomes differ in the sequence of the double stranded region but not the sequence of the single stranded overhang. In some embodiments, the reaction chamber comprises an enzyme that polymerizes the chemical units present therein to form the polymer. In some embodiments, the enzyme is selected from the group consisting of a ligase and a terminal deoxynucleotidyl transferase. In some embodiments, the enzyme is a ligase and the reaction chamber further comprises all reagents necessary to produce the polymer.

The presently disclosed subject matter also provides methods for creating a microorganism that is capable of exhibiting genetic memory by recording the time history of one or more an environmental state variable into genetic material stored within its cell or cells. In some embodiments, the nucleotide sequence of the stored genetic material can be expressed. In some embodiments, the recorded genetic material is incorporated into the genome of the microorganism and is inheritable by the offspring of the organism. In some embodiments, the nucleotide sequence of the stored genetic material can be expressed. In some embodiments, the method for recording the time history of one or more an environmental state variable into genetic material is a method disclosed herein. In some embodiments, the chemical sensing and recording mechanism is contained within a vesosome. In some embodiments, the vesosome is inserted into the microorganism using a pipette. In some embodiments, the vesosome is inserted into the microorganism using a lipofection. In some embodiments, the vesosome is inserted into an artificial organism as part of the process of assembling that microorganism. In some embodiments, the chemical and sensing recording mechanism is inserted into the microorganism using a pipette. In some embodiments, the chemical and sensing recording mechanism is inserted into the microorganism using lipofection. In some embodiments, the chemical and sensing recording mechanism is inserted into an artificial microorganism as part of the process of assembling that microorganism. In some embodiments, the vesosome is inserted into an artificial organism by including it a mixture or solution that is used to hydrate the lipid film that ultimately becomes the lipid bilayer which constitutes the cell membrane of the artificial organism.

In some embodiments, the methods for creating a microorganism that is capable of exhibiting genetic memory by recording the time history of one or more of an environmental state variable into genetic material stored within its cell or cells yields an organism which exhibits genetic memory. In some embodiments, the method for creating a microorganism that is capable of exhibiting genetic memory by recording the time history of one or more an environmental state variable into genetic material stored within its cell or cells yields an organism is possessed of a programmable genome.

The presently disclosed subject matter also provides compositions for implementing an organism that exhibits genetic memory. In some embodiments, the organism contains within it (a) one or a plurality of sensing and chemical recording mechanisms which record the time history of changes to one or more environmental state variables to which the microorganism is exposed into a strand of genetic material in such a way that the nucleotide sequence in the genetic material provides a record of the time series of those environmental variables; and (b) a chamber containing a mixture of enzymes and other reagents which are able to incorporate the strand of genetic material written by the chemical recording mechanism into the genetic material of the host microorganism by viral or other mechanisms. In some embodiments, the chemical sensing and recording mechanisms comprise: (a) a plurality of reservoirs each containing one or more chemical units, wherein each reservoir is characterized by a thermal stability point at or above which one or more of the chemical units present within the reservoir is released from the reservoir; (b) a reaction chamber in which the one or more chemical units that have been released collect; and (c) an enzyme and all other reagents necessary for polymerizing the one or more chemical units present in the reaction chamber to form a polymer. In some embodiments, the chemical sensing and recording mechanisms comprise: (a) a plurality of photosensitive liposomes each containing one or more chemical units, wherein each photosensitive liposome is characterized by a stability point governed by the intensity of light or electromagnetic radiation incident on the reservoir such that beyond a given threshold for said light intensity one or more of the chemical units present within the reservoir is released from the reservoir; (b) a reaction chamber in which the one or more chemical units that have been released collect; and (c) an enzyme and all other reagents necessary for polymerizing the one or more chemical units present in the reaction chamber to form a polymer. In some embodiments, the chemical sensing and recording mechanisms are contained within one or a plurality of vesosomes. In some embodiments, the vesosome is originally inserted into the microorganism using a pipette. In some embodiments, the vesosome is originally inserted into the microorganism using lipofection.

It is an object of the presently disclosed subject matter to provide methods for chemical recording of environmental state variables.

An object of the presently disclosed subject matter having been stated above, other objects and advantages will become apparent to those of ordinary skill in the art after a study of the following description of the presently disclosed subject matter and non-limiting Examples.

### BRIEF DESCRIPTION OF THE DRAWINGS

The patent or application file contains at least one drawing executed in color. Copies of this patent with color drawings(s) will be provided by the Patent and Trademark Office upon request and payment of necessary fee.

FIG. 1 depicts a representative nanoimprint process: (1) the substrate is covered with a polymer resist layer (polymer film), (2) the polymer is heated about its glass transition point, (3) the mold (rigid master) is pressed into the polymer, (4) the temperature is lowered, and (5) the mold removed (Gates et al., 2004)

FIG. 2 is a free-body diagram of a cantilever with a proof mass located at the end of the beam.

FIG. 3 is a free body diagram of a single cantilever modeled as a harmonic oscillator.

FIG. 4 is a series of Bode plots for a driven harmonic oscillator: (top) amplitude and (bottom) phase.

FIG. 5 is a free body diagram for a single cantilever modeled as a damped, driven harmonic oscillator.

FIG. 6 is a series of Bode plots for a damped, driven harmonic oscillator: (top) amplitude and (bottom) phase.

FIG. 7 is a plot of acceleration and cantilever length using the properties summarized in Value Set 1 of Table 2.

FIG. 8 is a plot of acceleration and cantilever length using the properties summarized in Value Set 2 of Table 2.

FIG. 9 is a plot of the acceleration (left) and corresponding cantilever position (right) data calculated from Value Set 3 of Table 5.

FIG. 10 is a plot of the acceleration (left) and corresponding cantilever position (right) data calculated from Value Set 3 of Table 6.

FIG. 11 is a schematic of the buoyancy driven concept with a high density region of chemical units and a low density region of chemical units. The arrows denote the direction of fluid flow.

FIG. 12 is depicts how if chemical unit A is in greater concentration than chemical unit B, concentration and velocity data forms three regions.

FIG. 13 is a depiction of the different temperature regions of an exemplary device. Gravity is noted and the resulting fluid flow is shown with the arrows.

FIG. 14 depicts the forces that can be experienced by the system. The track is modeled as a 1-D channel with repeating boundary conditions.

FIG. 15 depicts an energy balance for the system shown over an infinitesimal control volume with distance ds.

FIG. 16 depicts an exemplary concept overview that can be used to achieve the chemical documentation of the deflection of the cantilevers. As a particular acceleration is experienced, the corresponding cantilever will deflect and the assigned chemical unit, denoted with different colors here, will be released. These units could be strung together as a record of the acceleration and time data.

FIG. 17 depicts an exemplary data strand (left) where for a given acceleration, the cantilever deflects releasing the assigned unit as well as all cantilevers with accelerations smaller than the given acceleration resulting in a mix of units. In the right section of the figure is a sample graph of the accelerations experienced.

FIGS. 18A and 18B are examples of a DNA sticky end (FIG. 18A) and a DNA blunt end (FIG. 18B).

FIG. 19 is a plot showing that as the DNA sequence increases in base pairs, the time to diffuse 1 μm increases.

FIG. 20 is a plot displaying the increase in base pair length with the cumulative time. The sticky end scenario is shown as a solid, blue line and the blunt end scenario is shown as a dashed, green line.

FIG. 21 is a schematic of the device integrating mechanical sensing and chemical recording.

FIG. 22 is a block diagram for the device dynamics.

FIG. 23 is a diagram depicting the location of three exemplary points of interest of the device when determining the chemical dynamics.

FIG. 24 is a series of Bode plots for the chemical dynamics: (top) amplitude and (bottom) phase.

FIG. 25 is a series of Bode plots for the combined mechanical and chemical dynamics.

FIG. 26 is a modified block diagram of device dynamics that takes into account the valve dynamics.

FIG. 27 is a diagram depicting the location of three exemplary concentration points of interest of the device when determining the valve dynamics.

FIG. 28 is a series of Bode plots for the combined valve and chemical dynamics of the device: (top) amplitude and (bottom) phase.

FIG. 29 is a series of plots showing the system bandwidth and gain when different system parameters are varied. (top) A comparison of all varied parameters; (bottom left) an enlarged view of the number of base pairs in the DNA segment and (bottom right) the dimensions of the valve and volume of the mixing chamber.

FIG. 30 is a comparison of an exemplary device of the presently disclosed subject matter to current, conventional accelerometers. The relative size of the accelerometers is denoted by the size of the circle. The proposed device is represented by a star and its size is not to scale.

FIG. 31 is a series of plots of temperature profiles employed for testing the behaviors of two thermosensitive liposomes: Liposome A and Liposome B. The transition temperature for Liposome A was 50° C. and for Liposome B was 60° C.

FIGS. 32A-32D are a series of plots of temperature profiles for testing the behaviors of Liposomes A and B under various conditions. Axes: bottom−Strand Length*; Right−log CG/AT; Top−Time*; and Left−Temperature (° C.). *Non-dimensionalized.

FIGS. 32A and 32B depict the results of separately testing Liposome A and B, respectively, at their respective transition temperatures.

FIGS. 32C and 32D depict the results of testing the combination of Liposome A and B with two different temperature profiles. FIG. 32C shows data from experiments testing Liposome A and B at the lower transition temperature of A. FIG. 32D shows data from experiments testing Liposome A and B at both transition temperatures, along with two methods of adding ligase to the experiment

FIG. 33 is a schematic representation of an embodiment of the presently disclosed subject matter with respect to chemical recording of temperature as the environmental state variable as described in EXAMPLE 1.

FIG. 34 is a schematic representation of an embodiment of the presently disclosed subject matter with respect to chemical recording of temperature as the environmental state variable as described in the EXAMPLE 2.

FIG. 35 is a photographic image of a polyacrylamide gel from a single strand DNA ligation experiment described in EXAMPLE 2.

### DETAILED DESCRIPTION

### I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the articles “a”, “an”, and “the” refer to “one or more” when used in this application, including in the claims. For example, the phrase “a symptom” refers to one or more symptoms. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments, ±20%, in some embodiments, ±10%, in some embodiments, ±5%, in some embodiments, ±1%, in some embodiments, ±0.5%, and in some embodiments, ±0.1% from the specified amount, as such variations are appropriate to perform the disclosed method.

### II. Mechanical Sensing of Acceleration

Current accelerometers use a variety of methods to sense acceleration and are available in a variety of sizes. An encapsulated piezoresistive accelerometer with the smallest published volume of 0.034 mm³ and dimensions of 387 μm×387 μm×230 μm was recently fabricated (Park et al., 2006). An exemplary accelerometer as disclosed herein with the greatest dimension between 6-8 μm, is significantly smaller. In order to achieve the desired device package size, using electronics to sense or record the accelerations can be difficult. Therefore, in some embodiments, the presently disclosed devices rely on mechanical features such as cantilevers to sense acceleration.

One mechanism for sensing the acceleration mechanically is through a cantilever. A cantilever of a given mass and length deflects a corresponding distance when subjected to an appropriate acceleration. Through this action, a cantilever can be used to sense acceleration. The acceleration required to deflect a given distance is a₀. Any acceleration less than a₀ might deflect the cantilever some distance, but only an acceleration equal to or greater than a₀ deflects the cantilever completely. When complete deflection is achieved, the acceleration is recorded.

In some embodiments, an array of nanocantilevers is employed to measure a range of accelerations. In an array of cantilevers, if each cantilever has a unique mass and length, then each cantilever deflects at a given, unique acceleration, a₀, which for vertical situations, also includes gravity. The resolution and range of accelerations that can be detected can depend on the number and length of the cantilevers in the array. By varying these parameters and the material properties of the cantilevers, virtually any range and resolution of accelerations can be achieved.

II.A. Exemplary Fabrication Techniques

A variety of top-down fabrication techniques and processes have been developed for creating nano-sized mechanical features, a representative number of which are surveyed below. Each of these techniques can be employed to create nanocantilevers and/or arrays of nanocantilevers and a range of sizes can be achieved. These techniques have can also be employed in the fabrication of nanochannels, an alternative approach to nanocantilevers.

II.A.1 Typical Fabrication Processes—Nanocantilevers

Nanocantilevers can be fabricated using a variety of processes, but a typical process includes patterning the substrate, depositing the cantilever material onto the pattern, and releasing the cantilevers from the resist.

Metallic nanocantilevers can be fabricated using this basic method (see e.g., Luo & Chakraborty, 2005). The fabrication process disclosed in this reference involved spin coating a layer of 495K polymethyl methacrylate (PMMA) resist onto a silicon wafer and patterning it using electron beam lithography. A thin metallic layer was then deposited onto the PMMA using either sputtering or thermal evaporation and the pattern was transferred using a lift-off process. The nanocantilevers were released using deep reactive ion etching (DRIE). The cantilevers fabricated using this method were made from either aluminum or gold and measured about 40 nm thick, 200-300 nm wide, and 5 μm long.

A similar method was used to produce an array of “ultra-short” nanocantilevers that measured 50 nm thick, 150 nm wide, and 2 μm long with a pitch of approximately 500 nm (see Nilsson et al., 2003). The process disclosed in this reference involves a two layer resist system and electron beam lithography to create the pattern. Lift-off, deposition of a Cr layer, and release of the cantilevers is then performed.

II.A.2 Typical Fabrication Processes—Nanochannels

There are two typical fabrication processes for creating nanochannels. Bulk micromachining involves patterning the substrate, etching to create trenches, and closing the trenches using wafer bonding to attach a top layer (see Mijatovic et al., 2005). Another common method is surface machining which involves depositing the material desired for the nanochannel walls onto the substrate, followed by a sacrificial layer which is patterned, and a top layer of the nanochannel wall material which is also patterned (see Mijatovic et al., 2005). After the sacrificial layer is removed through etching, the nanochannel is formed. However, removing the sacrificial layer can be extremely time intensive. Another alternative is buried channels which was recently developed and uses a series of etching steps to create channels that are embedded in the substrate (see e.g., de Boer et al., 2000; Perry & Kandlikar, 2005).

II.B. Patterning Techniques

Several patterning techniques have been developed for nanofabrication including photolithography, electron beam lithography, and nanoimprint lithography. The resolution obtained in patterning determines the size of the feature created. Therefore, techniques that offer tighter resolutions will produce smaller cantilevers or channels.

II.B.1. Photolithography

Photolithography is commonly used in microfabrication. A mask of the desired pattern is prepared and placed onto a substrate that has a photoresist layer. Using ultraviolet light, the resist is exposed, transferring the mask pattern to the substrate. The resist is then etched leaving behind the exposed resist. The resolution of photolithography has continued to be improved in order to use it as a nanofabrication technique by improving the resist performance and adjusting exposure techniques. Linewidths of 50 nm have been reported (Itani et al., 2002).

II.B.2. Electron Beam Lithography

Electron beam lithography uses a high energy electron beam to expose resists that are sensitive to electrons such as PMMA. Unlike photolithography, no mask is required. The beam writes the desired pattern across the surface. While the beam can be extremely narrow, on the order of 0.5 nm, the ultimate resolution of the technique is on the order of 5 nm due to proximity effects. These effects are the result of scattered electrons exposing resist away from the point of impact (Madou, 2002). The process is typically very slow and expensive. The resolution limits of electron beam lithography for both isolated features and dense arrays of features have been explored (Vieu, 2000). For an isolated feature, linewidths between 3 and 5 nm were achieved and the pitch for arrays of features ranged from 20 to 50 nm depending on the type of feature being patterned. In addition to the smaller linewidths and tighter pitches, further processing such as lift-off and RIE were successfully completed.

II.B.3. Nanoimprint Lithography

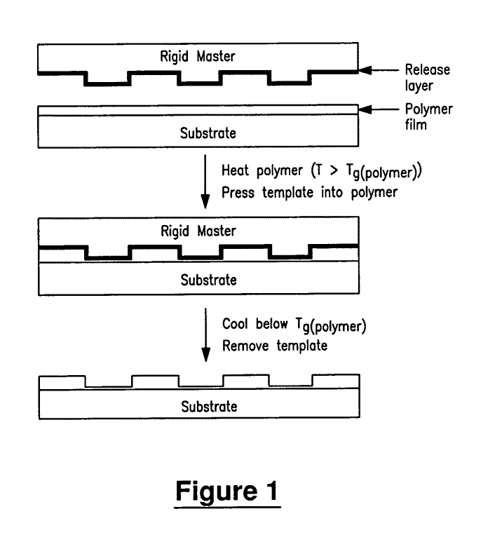

Nanoimprint lithography has been considered an unconventional fabrication technique. It was developed in the 1990s by Professor Chou and his lab at Princeton (Chou et al., 1996). The standard process (see FIG. 1) involves pressing a mold into a substrate that is coated with a polymer film. The film is heated to a temperature that allows the polymer to flow into the recesses of the mold and then cooled. The mold is removed and typically DRIE is used to remove the compressed areas of polymer. At this point, further processes can be done such as sputtering and liftoff. The mold with the nanofeatures is created using electron beam lithography and can be made for a range of materials including silicon, silicon dioxide, gallium arsenide, various metals, and ceramics.

Nanoimprint lithography is a high-throughput, low cost process because after the initial cost of creating the mold, the mold can be used many times patterning entire wafers. The method shows a lot of promise of becoming a manufacturing process.

Using nanoimprint lithography, a mold that had lateral features of 25 nm was created (Chou et al., 1996). PMMA was used as the resist. The recommended aspect ratio of the features is 3:1 and the resist layer should be thicker than the mold intrusion in order to prevent contact between the mold and the substrate and prolong the life of the mold. Using the standard process, a variety of structures were created including 25 nm diameter holes with a 120 nm pitch and 30 nm strips with a 70 nm pitch.

A variation of the nanoimprint process called photocurable nanoimprint lithography has been established (see Austin et al., 2004). In this variation, instead of varying the temperature to cure the polymer resist, a photocurable resist is cured using UV light. Using this process, a grating with 7 nm linewidth and 14 nm pitch was demonstrated. This grating is the thinnest linewidth and tightest pitch that has been reported using nanoimprint lithography.

II.C. Release and Lift-Off Techniques

Once a feature such as a cantilever has been patterned, it needs to be released from the substrate. Releasing is typically achieved through dry or wet etching of the sacrificial layer.

II.C.1. Deep Reactive Ion Etch

The deep reactive ion etch (DRIE) release technique is an anisotropic dry etching method (Madou, 2002; see also “Deep reactive ion etching” in Wikipedia). It has an etch rate of 5-10 μm/minute and is used to create high aspect ratio features. There are two typical processes. The first process involves cryogenically cooling the wafer to about 100° C. which prevents isotropic chemical etching and allows the ion bombardment to be highly directional. The alternative process is called the Bosch process and involves cycling between etching and passivation to achieve vertical etching.

II.C.2. Critical Point Drying

A critical point is the property for a given material where at a critical temperature, pressure, and volume, the liquid state and the gas state have the same density. As a result, a material can pass from the liquid phase to the gas phase without an abrupt change of state. This is beneficial because as a result there is no surface tension.

Water has a critical point at 374° C. and 3212 psi; carbon dioxide's critical point is at 31.1° C. and 1072 psi. Because of the lower temperature and pressure of the critical point, carbon dioxide (CO₂) is commonly used in critical point drying. In biological samples, in order to replace the water with carbon dioxide, an intermediate fluid is required. Intermediate fluids include acetone, ethanol, and Freon (“Critical point drying incorporating Emitech K850” (1999) Emitech Ltd.). The sample is flushed with the intermediate fluid and then the transitional fluid, CO₂. For fabrication, the same principle of an intermediate fluid can be used. The photoresist is etched with acetone and then replaced with CO₂ which is used in the critical point drying process.

II.D. Design of a Nanocantilever Array

In some embodiments, an array of nanocantilevers is employed to measure acceleration. In some embodiments, each nanocantilever has the same width and thickness but a different length, although all the dimensions of each cantilever can be variable. In some embodiments, only the length is varied. Because of the variation in dimensions, each nanocantilever has a unique mass, length, and stiffness, and deflects a given distance at a different acceleration, a₀.

II.D.1. Undamped

The undamped analysis of the cantilevers is applicable when the cantilever array is operated in air. As disclosed herein, the array was modeled two ways in this situation: constant acceleration and cyclic acceleration.

II.D.1.a. Constant Acceleration

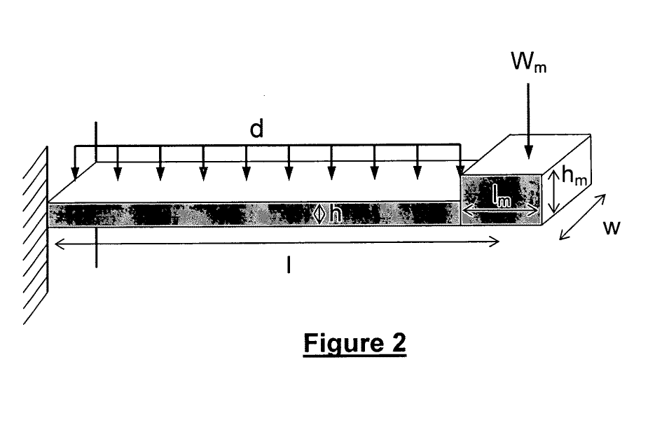

Using beam theory, cantilevers were modeled with a distributed load, d, due to the mass of the beam and a point mass, Wₘ, due to the addition of a proof mass at the end of the beam. A free-body diagram of a single cantilever is shown in FIG. 2.

The loads were defined as:

d=whρa₀

Wₘ=Mₚa₀=whₘlₘρa₀ (1)

where w and h are the width and thickness of the cantilever respectively, ρ is the density of the cantilever material, and Mₚ is the proof mass. The proof mass has dimensions of length (lₘ), width (w), and thickness (hₘ). Using these loads, the total deflection of the beam, Δ, is

$$Δ = \frac{d {L}^{4}}{8 EI} + \frac{{W}_{m} {L}^{3}}{3 EI} = \frac{3 d {L}^{4} + 8 {W}_{m} {L}^{3}}{24 EI} , \qquad ( 2 )$$

where E is the Young's Modulus and l is the moment of inertia defined as

$$I = \frac{{wh}^{3}}{12} , \qquad ( 3 )$$

for a rectangular cantilever geometry. Combining and simplifying Equations (1), (2), and (3) yields the equation for the deflection of the cantilever

$$Δ = \frac{3 {L}^{4} h ρ {a}_{0} + 8 {L}^{3} {h}_{m} {l}_{m} ρ {a}_{0}}{2 {Eh}^{3}} . \qquad ( 4 )$$

Solving this equation for a₀ gives

$${a}_{0} = \frac{2 Δ {Eh}^{3}}{3 h ρ {L}^{4} + 8 {h}_{m} {l}_{m} ρ {L}^{3}} , \qquad ( 5 )$$

which is the equation for the acceleration required to deflect a given cantilever a specified distance.

II.D.1.b. Harmonic Acceleration

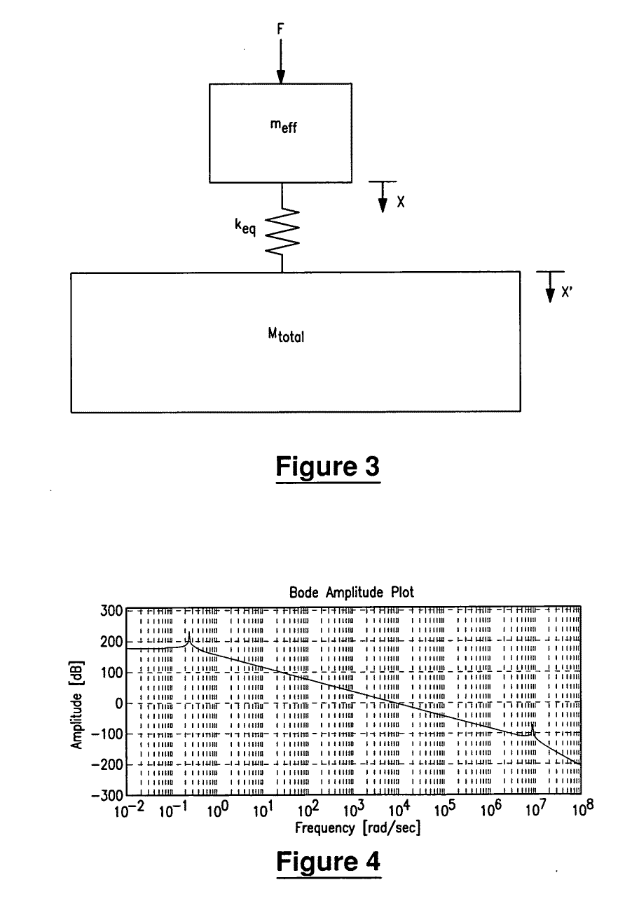

Using a sinusoidal input to achieve a harmonic acceleration, the cantilever is modeled as a driven harmonic oscillator. A free-body diagram is shown in FIG. 3.

The general equation for a driven harmonic oscillator is

$${m}_{eff} \frac{{d}^{2} x}{d {t}^{2}} + {k}_{eq} x = {F}_{d} = {k}_{eq} {x}^{′} . \qquad ( 6 )$$

It is an inhomogeneous differential equation where x is the position of the cantilever, the effective mass (m_{eff}) and spring constant (k_{eq}) are beam properties, and F_{d} is the force exerted on the cantilever. The position of the entire cantilever array with mass Mₜₒₜₐₗ is designated x′ and equal to

x′=A_{d} sin(ω_{d}t) (7),

where A_{d} is the driving amplitude and ω_{d} is the driving frequency. The driving acceleration, a_{d}, equals

$${a}_{d} = \frac{{kx}^{′}}{{m}_{eff}} . \qquad ( 8 )$$

The effective mass of a single cantilever is the distributed mass of the beam combined with the proof mass located at the end of the beam and is equal to

$${m}_{eff} = {M}_{p} + m + {M}_{p} ( \frac{{l}^{2}}{{l}_{m}^{2}} + \frac{l}{{l}_{m}} + \frac{1}{4} ) + m ( \frac{1}{3} + \frac{{h}^{2}}{12 {l}^{2}} ) . \qquad ( 9 )$$

The full derivation is as follows (equations not numbered): The effective mass of the cantilever and proof mass is calculated using the kinetic energy of the beam.

$$T = \frac{1}{2} M {\dot{x}}^{2} + \frac{1}{2} I {\dot{θ}}^{2} ,$$

where

M=Mₚ+m

I=I_{c}+Iₚ,

which upon substitution gives

$$T = \frac{1}{2} {M}_{p} {\dot{x}}^{2} + \frac{1}{2} m {\dot{x}}^{2} + \frac{1}{2} {I}_{p} {\dot{θ}}^{2} + \frac{1}{2} {I}_{c} {\dot{θ}}^{2} .$$

Using the parallel axis theorem:

I_{axisofrotation}=I_{masscenter}+md²

where d is the perpendicular distance between the axis of rotation and mass center, the mass moment of inertias are calculated.

$${I}_{c} = m ( \frac{{l}^{2} + {h}^{2}}{12} ) + m \frac{{l}^{2}}{4} = m ( \frac{{l}^{2} + {h}^{2}}{12} + \frac{{l}^{2}}{4} )$$

$${I}_{p} = {{M}_{p} ( l + \frac{{l}_{m}^{′}}{2} )}^{2}$$

### Substituting

$${\dot{θ}}_{c} = \frac{\dot{x}}{l}$$

$${\dot{θ}}_{p} = \frac{\dot{x}}{{l}_{m}}$$

results in the kinetic equation becoming

$$T = \frac{1}{2} {M}_{P} {\dot{x}}^{2} + \frac{1}{2} m {\dot{x}}^{2} + \frac{1}{2} {{I}_{p} ( \frac{\dot{x}}{{l}_{m}} )}^{2} + \frac{1}{2} {{I}_{c} ( \frac{\dot{x}}{l} )}^{2} .$$

Differentiating the energy gives a force

$$\frac{d T}{d t} = F = {M}_{p} \ddot{x} + m \ddot{x} + \frac{{I}_{p}}{{l}_{m}^{2}} \ddot{x} + \frac{{I}_{c}}{{l}^{2}} \ddot{x}$$

$$F = ( {M}_{p} + m + \frac{{I}_{p}}{{l}_{m}^{2}} + \frac{{I}_{c}}{{l}^{2}} ) \ddot{x} = {m}_{eff} \ddot{x}$$

which can be solved for the effective mass

$${m}_{eff} = {M}_{p} + m + \frac{{{M}_{p} ( l + \frac{{l}_{m}}{2} )}^{2}}{{l}_{m}^{2}} + \frac{m ( \frac{{l}^{2} + {h}^{2}}{12} + \frac{{l}^{2}}{4} )}{{l}^{2}}$$

$${m}_{eff}^{′} = {M}_{p} + m + {M}_{p} ( \frac{{l}^{2}}{{l}_{m}^{2}} + \frac{l}{{l}_{m}} + \frac{1}{4} ) + m ( \frac{1}{3} + \frac{{h}^{2}}{12 {l}^{2}} ) .$$

The spring constant of the beam is calculated from

$${k}_{eq} = \frac{{Eh}^{3} w}{4 {l}^{3}} . \qquad ( 10 )$$

Dividing Equation (6) by m_{eff} gives

$$\frac{{d}^{2} x}{d {t}^{2}} + {ω}_{n}^{2} x = {ω}_{n}^{2} {A}_{d} sin ( {ω}_{d} t ) , \qquad ( 11 )$$

where the natural frequency, ωₙ, is substituted and equals

$${ω}_{n} = \sqrt{\frac{{k}_{eq}}{{m}_{eff}}} . \qquad ( 12 )$$

Solving Equation (11) results in a general solution of

$$x ( t ) = {c}_{1} {e}^{- i {ω}_{n} t} + {c}_{2} {e}^{{iω}_{n} t} + \frac{{A}_{d} {ω}_{n}^{2}}{{ω}_{n}^{2} - {ω}_{d}^{2}} sin ( {ω}_{d} t ) . \qquad ( 13 )$$

Using the initial conditions

t=0,x=\dot{x}=\ddot{x}=0 (14),

the coefficients c₁ and c₂ can be solved and Equation (13) becomes

$$x ( t ) = \frac{{A}_{d} {ω}_{n} {ω}_{d}}{2 i ( {ω}_{n}^{2} - {ω}_{d}^{2} )} ( {e}^{- i {ω}_{n} t} - {e}^{i {ω}_{o} t} ) + \frac{{A}_{d} {ω}_{n}^{2}}{{ω}_{n}^{2} - {ω}_{d}^{2}} sin ( {ω}_{d} t ) , \qquad ( 15 )$$

which is the equation of motion for a single cantilever modeled as a driven harmonic oscillator.

The full derivation is as follows (equations not numbered): the general equation for a driven harmonic oscillator is

$${m}_{eff} \frac{{d}^{2} x}{d {t}^{2}} + {k}_{eq} x = {F}_{d} = {k}_{eq} {x}^{′}$$

where F_{d} is the input force and x′ is defined as

$${x}^{′} = {A}_{d} sin ( {ω}_{d} t )$$

$${a}_{d} = \frac{{k}_{eq} {x}^{′}}{{m}_{eff }}$$

where a_{d} is the driving acceleration, A_{d} is driving amplitude and ω_{d} is the driving frequency.
Dividing through by the effective mass and making the following substitution

$${ω}_{n} = \sqrt{\frac{{k}_{eq}}{{m}_{eff}}}$$

results in

$$\frac{{d}^{2} x}{d {t}^{2}} + {ω}_{n}^{2} x = {ω}_{n}^{2} {A}_{d} sin ( {ω}_{d} t ) .$$

Using Method of Undetermined Coefficients to solve the differential equation

\ddot{x}+ωₙ²x=ωₙ²A_{d} sin(ω_{d}t)

where the general solution is composed of a homogeneous solution (xₕ) and a particular solution (xₚ).

x(t)=xₕ+xₚ

The homogeneous solution is calculated

\ddot{x}ₕ+ωₙ²xₕ=0

λ²+ωₙ²=0

λ=±iωₙ

xₕ(t)=c₁e^{-iω}^{ₙ}^{t}+c₂e^{iω}^{ₙ}^{t}

The inhomogeneous solution is found

xₚ(t)=C cos(ω_{d}t)+D sin(ω_{d}t)

xₚ′(t)=−Cω_{d} sin(ω_{d}t)+Dω_{d} cos(ω_{d}t)

xₚ″(t)=−Cω_{d}² cos(ωt)−Dω_{d}² sin(ω_{d}t)

−Cω_{d}² cos(ω_{d}t)−Dω_{d}² sin(ω_{d}t)+ωₙ²C cos(ω_{d}t)+ωₙ²D sin(ω_{d}t)=ωₙ²A_{d} sin(ω_{d}t)

[−Cω_{d}²+ωₙ²C] cos(ω_{d}t)=[ωₙ²A_{d}+Dω_{d}²−ωₙ²D] sin(ω_{d}t)

In order to be true for all t, the coefficient on both sides must be zero.

$$- C {ω}_{d}^{2} + C {ω}_{n}^{2} = 0$$

$$C = 0 ;$$

$$or$$

$${ω}_{d}^{2} = {ω}_{n}^{2}$$

$${A}_{d} {ω}_{n}^{2} + D {ω}_{d}^{2} - D {ω}_{n}^{2} = 0$$

$$D = \frac{{A}_{d} {ω}_{n}^{2}}{{ω}_{n}^{2} - {ω}_{d}^{2}} \\ ⇒ {ω}_{n}^{2} \neq {ω}_{d}^{2}$$

$${x}_{p} ( t ) = \frac{{A}_{d} {ω}_{n}^{2}}{{ω}_{n}^{2} - {ω}_{d}^{2}} sin ( {ω}_{d} t )$$

The solution is

$$x ( t ) = {c}_{1} {e}^{- i {ω}_{n} t} + {c}_{2} {e}^{i {ω}_{n} t} + \frac{{A}_{d} {ω}_{n}^{2}}{{ω}_{n}^{2} - {ω}_{d}^{2}} sin ( {ω}_{d} t )$$

Using the initial conditions to solve for the coefficients c₁ and c₂

$$t = 0 , x = \dot{x} = \ddot{x} = 0$$

$$x ( t ) = {c}_{1} {e}^{- i {ω}_{n} t} + {c}_{2} {e}^{i {ω}_{n} t} + \frac{{A}_{d} {ω}_{n}^{2}}{{ω}_{n}^{2} - {ω}_{d}^{2}} sin ( {ω}_{d} t )$$

$$x ( 0 ) = {c}_{1} + {c}_{2} = 0$$

$${c}_{1} = - {c}_{2}$$

$$\dot{x} ( t ) = - {c}_{1} i {ω}_{n} {e}^{- i {ω}_{n} t} + {c}_{2} i {ω}_{n} {e}^{i {ω}_{n} t} + \frac{{A}_{d} {ω}_{n}^{2} {ω}_{d}}{{ω}_{n}^{2} - {ω}_{d}^{2}} cos ( {ω}_{d} t )$$

$$\dot{x} ( 0 ) = - {c}_{1} i {ω}_{n} + {c}_{2} i {ω}_{n} + \frac{{A}_{d} {ω}_{n}^{2} {ω}_{d}}{{ω}_{n}^{2} - {ω}_{d}^{2}} = 0$$

Combining the equations

$${c}_{2} i {ω}_{n} + {c}_{2} i {ω}_{n} + \frac{{A}_{d} {ω}_{n}^{2} {ω}_{d}}{{ω}_{n}^{2} - {ω}_{d}^{2}} = 0$$

$$2 {c}_{2} i {ω}_{n} = \frac{- {A}_{d} {ω}_{n}^{2} {ω}_{d}}{{ω}_{n}^{2} - {ω}_{d}^{2}}$$

$${c}_{2} = \frac{- {A}_{d} {ω}_{n} {ω}_{d}}{2 i ( {ω}_{n}^{2} - {ω}_{d}^{2} )}$$

$${c}_{1} = \frac{{A}_{d} {ω}_{n} {ω}_{d}}{2 i ( {ω}_{n}^{2} - {ω}_{d}^{2} )}$$

results in the final solution

$$x ( t ) = \frac{{ω}_{n} {A}_{d} {ω}_{d}}{2 i ( {ω}_{n }^{2} - {ω}_{d}^{2} )} ( {e}^{- i {ω}_{n} t} - {e}^{i {ω}_{n} t} ) + \frac{{ω}_{n}^{2} {A}_{d}}{( {ω}_{n}^{2} - {ω}_{d}^{2} )} sin ( {ω}_{d} t )$$

which is the equation of motion for a single cantilever modeled as a driven harmonic oscillator.

The transfer function for an undamped, driven harmonic oscillator described in Equation (11) is

$$X ( s ) = \frac{{ω}_{n}^{2} {A}_{d} {ω}_{d}}{( {s}^{2} + {ω}_{n}^{2} ) ( {s}^{2} + {ω}_{d}^{2} )} \qquad ( 16 )$$

and can be used to obtain the Bode plots shown in FIG. 4.

II.D.2. Damped

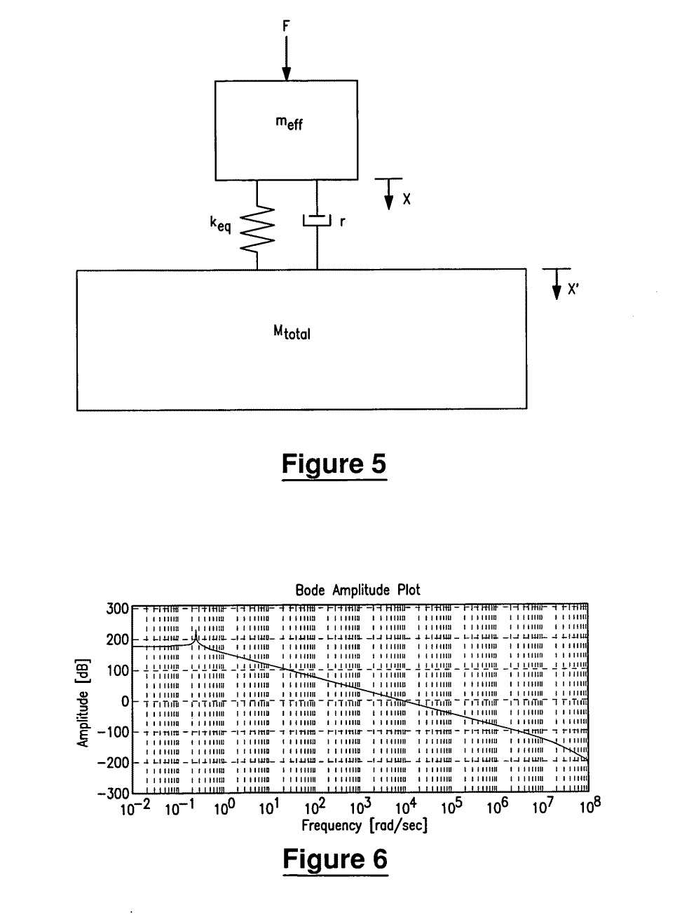

The damped analysis of the cantilevers an be employed to analyze beam deflections when the array is operated in fluid. Using a cyclic acceleration approach, the cantilever can be modeled as a driven, damped harmonic oscillator. A free-body diagram is shown in FIG. 5.

The general equation for a damped, driven harmonic oscillator is

$${m}_{eff} \frac{{d}^{2} x}{d {t}^{2}} + r \frac{d x}{d t} + {k}_{eq} x = {F}_{d} = {k}_{eq} {x}^{′} , \qquad ( 17 )$$

where r is the damping coefficient. The equation is inhomogeneous due to the driving force.
The following substitutions were made

$$2 ζ {ω}_{n} = \frac{r}{{m}_{eff}} \\ {ω}_{n} = \sqrt{\frac{{k}_{eq}}{{m}_{eff}}} \qquad ( 18 )$$

into Equation (17), which results in

\ddot{x}+2ζωₙ\dot{x}+ωₙ²x=ωₙ²A_{d} sin(ω_{d}t) (19).

Solving this equation results in a general solution of

$$x ( t ) = {c}_{1} {e}^{( - ζ {ω}_{n} + \sqrt{{( ζ {ω}_{n} )}^{2} - {ω}_{n}^{2}} ) t} + {c}_{2} {e}^{( - ζ {ω}_{n} - \sqrt{{( ζ {ω}_{n} )}^{2} - {ω}_{n}^{2}} ) t} + \frac{2 ζ {ω}_{n}^{3} {A}_{d} {ω}_{d}}{( {ω}_{d}^{4} - 2 {ω}_{o}^{2} {ω}^{2} + 4 {ζ}^{2} {ω}_{n}^{2} {ω}_{d}^{2} + {ω}_{n}^{4} )} cos ( {ω}_{d} t ) - \frac{{ω}_{n}^{2} {A}_{d} ( {ω}_{d}^{2} - {ω}_{n}^{2} )}{( {ω}_{d}^{4} - 2 {ω}_{o}^{2} {ω}^{2} + 4 {ζ}^{2} {ω}_{n}^{2} {ω}_{d}^{2} + {ω}_{n}^{4} )} sin ( {ω}_{d} t ) . \qquad ( 20 )$$

Using the same initial conditions as the undamped case, Equation (14), the coefficients c₁ and c₂ can be solved and Equation (20) becomes

$$x ( t ) = ( \begin{matrix}\frac{\begin{matrix}2 ζ {ω}_{n}^{3} {A}_{d} {ω}_{d} ( ζ {ω}_{n} - \sqrt{{( ζ {ω}_{n} )}^{2} - {ω}_{n}^{2}} ) - \\ {ω}_{n}^{2} {A}_{d} ( {ω}_{d}^{2} - {ω}_{n}^{2} ) {ω}_{d}\end{matrix}}{\begin{matrix}( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} ) \\ ( - 2 \sqrt{{( ζ {ω}_{n} )}^{2} - {ω}_{n}^{2}} )\end{matrix}} - \\ \frac{2 {ζω}_{n}^{3} {A}_{d} {ω}_{d}}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )}\end{matrix} ) {e}^{( - ζ {ω}_{n} + \sqrt{{( ζ {ω}_{n} )}^{2} - {ω}_{n}^{2}} ) t} - ( \frac{\begin{matrix}2 ζ {ω}_{n }^{3} {A}_{d} {ω}_{d} ( ζ {ω}_{n} - \sqrt{{( {ζω}_{n} )}^{2} - {ω}_{n}^{2}} ) - \\ {ω}_{n}^{2} {A}_{d} ( {ω}_{d}^{2} - {ω}_{n}^{2} ) {ω}_{d}\end{matrix}}{\begin{matrix}( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} ) \\ ( - 2 \sqrt{{( ζ {ω}_{n} )}^{2} - {ω}_{n}^{2}} )\end{matrix}} ) {e}^{( - ζ {ω}_{n} - \sqrt{{( ζ {ω}_{n} )}^{2} - {ω}_{n}^{2}} ) t} + \frac{2 ζ {ω}_{n}^{3} {A}_{d} {ω}_{d}}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )} cos ( {ω}_{d} t ) - \frac{{ω}_{n}^{2} {A}_{d} ( {ω}_{d}^{2} - {ω}_{n}^{2} )}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )} sin ( {ω}_{d} t ) \qquad ( 21 )$$

which is the equation of motion for a single cantilever modeled as a damped, driven harmonic oscillator.

The full derivation is as follows (equations not numbered): the general equation for a damped driven harmonic oscillator is

$${m}_{eff} \frac{{d}^{2} x}{d {t}^{2}} + r \frac{d x}{d t} + {k}_{eq} x = {F}_{d} = {k}_{eq} {x}^{′}$$

where F_{d} is the input force and x′ is defined as

$${x}^{′} = {A}_{d} sin ( {ω}_{d} t )$$

$${a}_{d} = \frac{{k}_{eq} {x}^{′}}{{m}_{eff}}$$

where a_{d} is the driving acceleration, A_{d} is driving amplitude and ω_{d} is the driving frequency.
Dividing through by the effective mass

$$\frac{{d}^{2} x}{d {t}^{2}} + \frac{r}{{m}_{eff}} \frac{d x}{d t} + \frac{{k}_{eq}}{{m}_{eff}} x = \frac{{k}_{eq}}{{m}_{eff}} {x}^{′}$$

$$\frac{{d}^{2} x}{d {t}^{2}} + 2 ζ {ω}_{n} \frac{d x}{d t} + {ω}_{n}^{2} x = {ω}_{n}^{2} {A}_{d} sin ( {ω}_{d} t )$$

where the following substitutions are made

$$2 ζ {ω}_{n} = \frac{r}{{m}_{eff}}$$

$${ω}_{n} = \sqrt{\frac{{k}_{eq}}{{m}_{eff}}}$$

Using Method of Undetermined Coefficients to solve the differential equation

\ddot{x}+2ζωₙ\dot{x}+ωₙ²x=ωₙ²A_{d} sin(ω_{d}t)

where the general solution is composed of a homogeneous solution (xₕ) and a particular solution (xₚ).

x(t)=xₕ+xₚ

The homogeneous solution is calculated

$${\ddot{x}}_{h} + 2 ζ {ω}_{n} {\dot{x}}_{h} + {ω}_{n}^{2} {x}_{h} = 0$$

$${λ}^{2} + 2 ζ {ω}_{n} λ + {ω}_{n}^{2} = 0$$

$$λ = \frac{- 2 {ζω}_{n} \pm \sqrt{{( 2 {ζω}_{n} )}^{2} - 4 {ω}_{n}^{2}}}{2} = - ζ {ω}_{n} \pm \sqrt{{( ζ {ω}_{n} )}^{2} - {ω}_{n}^{2}}$$

$${x}_{h} ( t ) = {c}_{1} {e}^{( - {ζω}_{n} + \sqrt{{( {ζω}_{n} )}^{2} - {ω}_{n}^{2}} ) t} + {c}_{2} {e}^{( - {ζω}_{n} - \sqrt{{( {ζω}_{n} )}^{2} - {ω}_{n}^{2}} ) t}$$

The inhomogeneous solution is found

$${x}_{p} ( t ) = E cos ( {ω}_{d} t ) + F sin ( {ω}_{d} t )$$

$${\dot{x}}_{p} ( t ) = - E {ω}_{d} sin ( {ω}_{d} t ) + F {ω}_{d} cos ( {ω}_{d} t )$$

$${\ddot{x}}_{p} ( t ) = - E {ω}_{d}^{2} cos ( {ω}_{d} t ) - F {ω}_{d}^{2} sin ( {ω}_{d} t ) \\ - E {ω}_{d}^{2} cos ( {ω}_{d} t ) - F {ω}_{d}^{2} sin ( {ω}_{d} t ) - E 2 ξ {ω}_{n} {ω}_{d} sin ( {ω}_{d} t ) + F 2 {ξω}_{n} {ω}_{d} cos ( {ω}_{d} t ) + E {ω}_{n}^{2} cos ( {ω}_{d} t ) + F {ω}_{n}^{2} sin ( {ω}_{d} t ) = {ω}_{n}^{2} {A}_{d} sin ( {ω}_{d} t ) \\ [ - E {ω}_{d}^{2} + F 2 ξ {ω}_{n} {ω}_{d} + E {ω}_{n}^{2} ] cos ( {ω}_{d} t ) = [ \begin{matrix}{ω}_{n}^{2} {A}_{d} + F {ω}_{d}^{2} + \\ E 2 {ξω}_{n} {ω}_{d} - F {ω}_{n}^{2}\end{matrix} ] sin ( {ω}_{d} t )$$

In order to be true for all t, the coefficient on both sides must be zero.

$$- E {ω}_{d}^{2} + 2 F {ζω}_{n} {ω}_{d} + E {ω}_{n}^{2} = 0$$

$${ω}_{n}^{2} {A}_{d} + F {ω}_{d}^{2} + 2 E ζ {ω}_{n} {ω}_{d} - F {ω}_{n}^{2} = 0$$

$$E = \frac{2 F {ζω}_{n} {ω}_{d}}{{ω}_{d}^{2} - {ω}_{n}^{2}}$$

$${ω}_{n}^{2} {A}_{d} + F {ω}_{d}^{2} + \frac{4 F {ζ}^{2} {ω}_{n}^{2} {ω}_{d}^{2}}{{ω}_{d}^{2} - {ω}_{n}^{2}} - F {ω}_{n}^{2} = 0$$

$${ω}_{n}^{2} {A}_{d} + F ( {ω}_{d}^{2} + \frac{4 {ζ}^{2} {ω}_{n}^{2} {ω}_{d}^{2}}{{ω}_{d}^{2} - {ω}_{n}^{2}} - {ω}_{n}^{2} ) = 0$$

$${ω}_{n}^{2} {A}_{d} + F ( \frac{{ω}_{d}^{4} - 2 {ω}_{n}^{2} {ω}_{d}^{2} + 4 {ζ}^{2} {ω}_{n}^{2} {ω}_{d}^{2} + {ω}_{n}^{4}}{{ω}_{d}^{2} - {ω}_{n}^{2}} ) = 0$$

$$F = \frac{- {ω}_{n}^{2} {A}_{d} ( {ω}_{d}^{2} - {ω}_{n}^{2} )}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )}$$

$$E = \frac{2 ζ {ω}_{n}^{3} {A}_{d} {ω}_{d}}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )}$$

$${x}_{p} ( t ) = \frac{2 ζ {ω}_{n}^{3} {A}_{d} {ω}_{d}}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )} cos ( {ω}_{d} t ) - \frac{{ω}_{n}^{2} {A}_{d} ( {ω}_{d}^{2} - {ω}_{n}^{2} )}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )} sin ( {ω}_{d} t )$$

The solution is

$$x ( t ) = {c}_{1} {e}^{( - ζ {ω}_{n} + \sqrt{{( {ζω}_{n} )}^{2} - {ω}_{n}^{2}} ) t} + {c}_{2} {e}^{( - ζ {ω}_{n} - \sqrt{{( ζ {ω}_{n} )}^{2} - {ω}_{n}^{2}} ) t} + \frac{2 ζ {ω}_{n}^{3} {A}_{d} {ω}_{d}}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )} cos ( {ω}_{d} t ) - \frac{{ω}_{n}^{2} {A}_{d} ( {ω}_{d}^{2} - {ω}_{n}^{2} )}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )} sin ( {ω}_{d} t )$$

Using the initial conditions to solve for the coefficients c₁ and c₂, t=0, x=\dot{x}=\ddot{x}=0

$$x ( t ) = {c}_{1} {e}^{( - ζ {ω}_{n} + \sqrt{{( {ζω}_{n} )}^{2} - {ω}_{n}^{2}} ) t} + {c}_{2} {e}^{( - ζ {ω}_{n} - \sqrt{{( ζ {ω}_{n} )}^{2} - {ω}_{n}^{2}} ) t} + \frac{2 ζ {ω}_{n}^{3} {A}_{d} {ω}_{d}}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )} cos ( {ω}_{d} t ) - \frac{{ω}_{n}^{2} {A}_{d} ( {ω}_{d}^{2} - {ω}_{n}^{2} )}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )} sin ( {ω}_{d} t )$$

$$x ( 0 ) = {c}_{1} + {c}_{2} + \frac{2 ζ {ω}_{n}^{3} {A}_{d} {ω}_{d}}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )} = 0$$

$${c}_{1} = - {c}_{2} - \frac{2 ζ {ω}_{n}^{3} {A}_{d} {ω}_{d}}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )} = 0$$

$$\dot{x} ( t ) = - {c}_{1} ( ζ {ω}_{n} - \sqrt{{( ζ {ω}_{n} )}^{2} - {ω}_{n}^{2}} ) {e}^{( - ζ {ω}_{n} + \sqrt{{( ζ {ω}_{n} )}^{2} - {ω}_{n}^{2}} ) t} - {c}_{2} ( ζ {ω}_{n} + \sqrt{{( ζ {ω}_{n} )}^{2} - {ω}_{n}^{2}} ) {e}^{( - ζ {ω}_{n} - \sqrt{{( ζ {ω}_{n} )}^{2} - {ω}_{n}^{2}} ) t} - \frac{2 {ζω}_{n}^{3} {A}_{d} {ω}_{d}^{2}}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )} sin ( {ω}_{d} t ) - \frac{{ω}_{n}^{2} {A}_{d} ( {ω}_{d}^{2} - {ω}_{n}^{2} ) {ω}_{d}}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )} cos ( {ω}_{d} t )$$

$$\dot{x} ( 0 ) = - {c}_{1} ( ζ {ω}_{n} - \sqrt{{( {ζω}_{n} )}^{2} - {ω}_{n}^{2}} ) - {c}_{2} ( ζ {ω}_{n} + \sqrt{{( {ζω}_{n} )}^{2} - {ω}_{n}^{2}} ) - \frac{{ω}_{n}^{2} {A}_{d} ( {ω}_{d}^{2} - {ω}_{n}^{2} ) {ω}_{d}}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )} = 0$$

Combining the equations:

$$- ( ζ {ω}_{n} - \sqrt{{( {ζω}_{n} )}^{2} - {ω}_{n}^{2}} ) ( - {c}_{2} - \frac{2 {ζω}_{n}^{3} {A}_{d} {ω}_{d}}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )} ) - {c}_{2} ( ζ {ω}_{n} + \sqrt{{( {ζω}_{n} )}^{2} - {ω}_{n}^{2}} ) - \frac{{ω}_{n}^{2} {A}_{d} ( {ω}_{d}^{2} - {ω}_{n}^{2} ) {ω}_{d}}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )} = 0$$

$${c}_{2} ( ζ {ω}_{n} - \sqrt{{( {ζω}_{n} )}^{2} - {ω}_{n}^{2}} - {ζω}_{n} - \sqrt{{( {ζω}_{n} )}^{2} - {ω}_{n}^{2}} ) = \frac{- 2 {ζω}_{n}^{3} {A}_{d} {ω}_{d}}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )} ( ζ {ω}_{n} - \sqrt{{( {ζω}_{n} )}^{2} - {ω}_{n}^{2}} ) + \frac{{ω}_{n}^{2} {A}_{d} ( {ω}_{d}^{2} - {ω}_{n}^{2} ) {ω}_{d}}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )}$$

$${c}_{2} ( - 2 \sqrt{{( {ζω}_{n} )}^{2} - {ω}_{n}^{2}} ) = \frac{- 2 ζ {ω}_{n}^{3} {A}_{d} {ω}_{d} ( ζ {ω}_{n} - \sqrt{{( {ζω}_{n} )}^{2} - {ω}_{n}^{2}} ) + {ω}_{n}^{2} {A}_{d} ( {ω}_{d}^{2} - {ω}_{n}^{2} ) {ω}_{d}}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )}$$

$${c}_{2} = \frac{- 2 ζ {ω}_{n}^{3} {A}_{d} {ω}_{d} ( ζ {ω}_{n} - \sqrt{{( {ζω}_{n} )}^{2} - {ω}_{n}^{2}} ) + {ω}_{n}^{2} {A}_{d} ( {ω}_{d}^{2} - {ω}_{n}^{2} ) {ω}_{d}}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} ) ( - 2 \sqrt{{( {ζω}_{n} )}^{2} - {ω}_{n}^{2}} )}$$

$${c}_{1} = \frac{2 ζ {ω}_{n}^{3} {A}_{d} {ω}_{d} ( ζ {ω}_{n} - \sqrt{{( {ζω}_{n} )}^{2} - {ω}_{n}^{2}} ) - {ω}_{n}^{2} {A}_{d} ( {ω}_{d}^{2} - {ω}_{n}^{2} ) {ω}_{d}}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} ) ( - 2 \sqrt{{( {ζω}_{n} )}^{2} - {ω}_{n}^{2}} )} - \frac{2 {ζω}_{n}^{3} {A}_{d} {ω}_{d}}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )}$$

results in the final solution:

$$x ( t ) = ( \begin{matrix}\frac{2 ζ {ω}_{n}^{3} {A}_{d} {ω}_{d} ( ζ {ω}_{n} - \sqrt{{( {ζω}_{n} )}^{2} - {ω}_{n}^{2}} ) - {ω}_{n}^{2} {A}_{d} ( {ω}_{d}^{2} - {ω}_{n}^{2} ) {ω}_{d}}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} ) ( - 2 \sqrt{{( {ζω}_{n} )}^{2} - {ω}_{n}^{2}} )} - \\ \frac{2 {ζω}_{n}^{3} {A}_{d} {ω}_{d}}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )}\end{matrix} ) {e}^{( - ζ {ω}_{n} + \sqrt{{( ζ {ω}_{n} )}^{2} - {ω}_{n}^{2}} ) t} - ( \frac{2 ζ {ω}_{n}^{3} {A}_{d} {ω}_{d} ( ζ {ω}_{n} - \sqrt{{( {ζω}_{n} )}^{2} - {ω}_{n}^{2}} ) - {ω}_{n}^{2} {A}_{d} ( {ω}_{d}^{2} - {ω}_{n}^{2} ) {ω}_{d}}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} ) ( - 2 \sqrt{{( {ζω}_{n} )}^{2} - {ω}_{n}^{2}} )} ) {e}^{( - ζ {ω}_{n} - \sqrt{{( ζ {ω}_{n} )}^{2} - {ω}_{n}^{2}} ) t} + \frac{2 ζ {ω}_{n}^{3} {A}_{d} {ω}_{d}}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )} cos ( {ω}_{d} t ) - \frac{{ω}_{n}^{2} {A}_{d} ( {ω}_{d}^{2} - {ω}_{n}^{2} )}{( {ω}_{d}^{4} - 2 {ω}_{n}^{2} {ω}_{d}^{2} + 4 {ζ}^{2} {ω}_{n}^{2} {ω}_{d}^{2} + {ω}_{n}^{4} )} sin ( {ω}_{d} t )$$

which is the equation of motion for a single cantilever modeled as a damped, driven harmonic oscillator.

The transfer function for an undamped, driven harmonic oscillator described by Equation (19) is

$$X ( s ) = \frac{{ω}_{n}^{2} {A}_{d} {ω}_{d}}{( {s}^{2} + 2 {ξω}_{n} s + {ω}_{n}^{2} ) ( {s}^{2} + {ω}_{d}^{2} )} \qquad ( 22 )$$

and can be used to obtain the Bode plots shown in FIG. 6.

II.D.3. Stiction

Stiction is a common problem in MEMS devices, particularly during fabrication. The micro- or nano-structure is drawn down to the substrate via capillary forces either during fabrication or operation of the device and does not release. As a result, the device's operation can be compromised. Equations for the critical length of cantilevers during fabrication and operation have been developed (Tas et al., 1996). Cantilevers with lengths greater than these critical lengths will suffer from stiction. For a cantilever deflected at its tip by an acceleration, the critical length is

$${l}_{crit} = \sqrt[4]{\frac{{gt}^{2} E}{4 n ρ a}} , \qquad ( 23 )$$

where g is gap spacing, t is the thickness of the cantilever, n is the number of times its own weight the cantilever is loaded, and a is the acceleration. For gold cantilevers undergoing maximum accelerations of 100 m/s², the critical length is 21.3 μm. Therefore, as long as GOLD cantilevers do not exceed this length, stiction should not be an issue.

II.E. Materials

A range of materials were considered for the nanocantilevers. Table 1 summarizes the various thin-film material properties of exemplary materials.

TABLE 1
Thin-film Material Properties of Potential Nanocantilever Materials^{a}

| | E | ρ | |
|---|---|---|---|
| | (GPa) | (kg/m³) | E/ρ |
| Si | 125-180 | 2330 | ~0.071 |
| SiO₂ | 70 | 2200 | 0.032 |
| Al | 70 | 2700 | 0.026 |
| GaAs | 85 | 5320 | 0.016 |
| Au | 70 | 19300 | 0.004 |
| Ti | 120 | 4500 | 0.027 |
| Si₃N₄ | 250 | 3100-3300 | ~0.061 |
| W | | 19250 | |
| SiC | 400 | 3300 | 0.13 |
| Ni | 200 | 8900 | 0.022 |
| Cr | 140 | 7150 | 0.0195 |

^{a}As set forth in “Material Index” (2006) MEMS and Nanotechnology Clearinghouse

The chosen material is ideally dense and flexible (a low E/ρ value) in order to maximize the weight of the cantilever while allowing the cantilever to deflect under reasonable accelerations. In some embodiments, the material is also inexpensive to obtain and commonly used in nanofabrication. In some embodiments, the material is gold, which has an E/ρ value of 0.004.

II.F. Simulation and Results

Using the models previously developed, material properties selected, and varying the beam parameters, the following results were obtained.

II.F.1. Undamped

Simulations of the two undamped models, constant acceleration and harmonic acceleration resulting from a sinusoidal input, were conducted and the results are summarized in the following sections.

II.F.1.a. Constant Acceleration

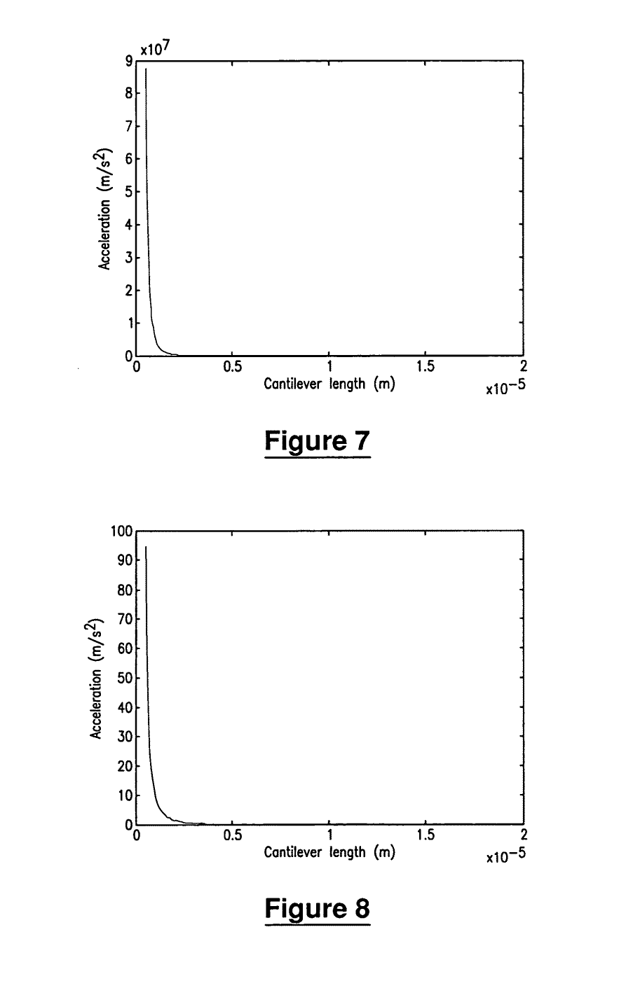

Using Equation (5) and the material and beam properties summarized in Value Set 1 of Table 2, the plot shown in FIG. 7 demonstrates the exponential decay of the acceleration as the cantilever lengths are increased.

TABLE 2
Material and Beam Properties Used to
Calculate the Acceleration Required

| Property | Value Set 1 | | Value Set 2 | |
|---|---|---|---|---|
| Material | Gold | | Gold | |
| E | 79 × 10⁹ | Pa | 79 × 10⁹ | Pa |
| P | 19300 | kg/m3 | 19300 | kg/m3 |
| L | 500 nm-20 μm | | 500 nm-20 μm | |
| H | 10 | nm | 10 | nm |
| W | 30 | nm | 30 | nm |
| Δ | 20 | nm | 20 | nm |
| proofmass | 0 | kg | 1 × 10⁻¹² | kg |

Table 3 summarizes a few acceleration values, cantilever lengths, and corresponding potential applications for devices with similar acceleration thresholds.

TABLE 3
Cantilever Lengths with Corresponding Accelerations
and Potential Applications

| Beam | | | |
|---|---|---|---|
| Length | Acceleration | | Typical Range |
| (nm) | (m/s²) | Application | (g's) |
| 2250 | 212,950 | Shock/Impact | 2000-20,000 |
| 4050 | 20,286 | Crash Testing | 2000 |
| 10,200 | 504 | Gun Firing | 20-100 |
| 15,300 | 100 | Aircraft | 1-10 |
| 20,000 | 34 | Vehicle Motion | 1-10 |
| N/A | 10 | Human Motion | 1-2 |

Using the same model and parameters, with the exception of adding a proof mass at the end of the cantilevers, Value Set 2 of Table 2, the data depicted in FIG. 8 was obtained.

With the addition of the proof mass, accelerations between 10 to 100 m/s² can be achieved with relatively short cantilevers. However, a proof mass of 1×10⁻¹² kg is significantly larger than the mass of a 1 μm cantilever, which is 5.79×10⁻¹⁸ kg. There thus can be a tradeoff between the size of the proof mass used and the length of the cantilevers. Table 4 gives a sampling of proof masses and the resulting range in cantilever lengths required to have accelerations between 10-100 m/s².

TABLE 4
Range of Cantilevers Required to Achieve 10-100
m/s2 accelerations with Addition of a Proof Mass

| Proof | Cantilever |
|---|---|
| Mass | Lengths |
| (kg) | (nm) |
| 1 × 10⁻¹² | 500-1050 |
| 1 × 10⁻¹³ | 1050-2250 |
| 1 × 10⁻¹⁴ | 2250-4900 |
| 1 × 10⁻¹⁵ | 4900-10,500 |
| 1 × 10⁻¹⁶ | 10,500-20,000 |

This tradeoff can be considered when selecting the length of the cantilevers and size of the proof mass in terms of the difficulty of fabrication and final package size.

II.F.1.b. Harmonic Acceleration

Using Equation (15) with the material and beam properties summarized in Value Set 3 of Table 5, the harmonic acceleration and resulting position data depicted in FIG. 9 were obtained.

TABLE 5
Material and Beam Properties Used to
Calculate the Acceleration Required

| Property | Value Set 3 | |
|---|---|---|
| Material | Gold | |
| E | 79 × 10⁹ | Pa |
| ρ | 19300 | kg/m³ |
| l | 500 nm-20 μm | |
| h | 10 | nm |
| w | 30 | nm |
| Δ | 20 | nm |
| proofmass | 0 | kg |

| | |
|---|---|
| A_{d} | 2.5 × 10⁻⁸ |
| ω_{d} | 2π |
| r | 5 × 10⁻⁴ |

The cantilever reaches 20 nm of deflection in 0.15 seconds.

II.F.2. Damped

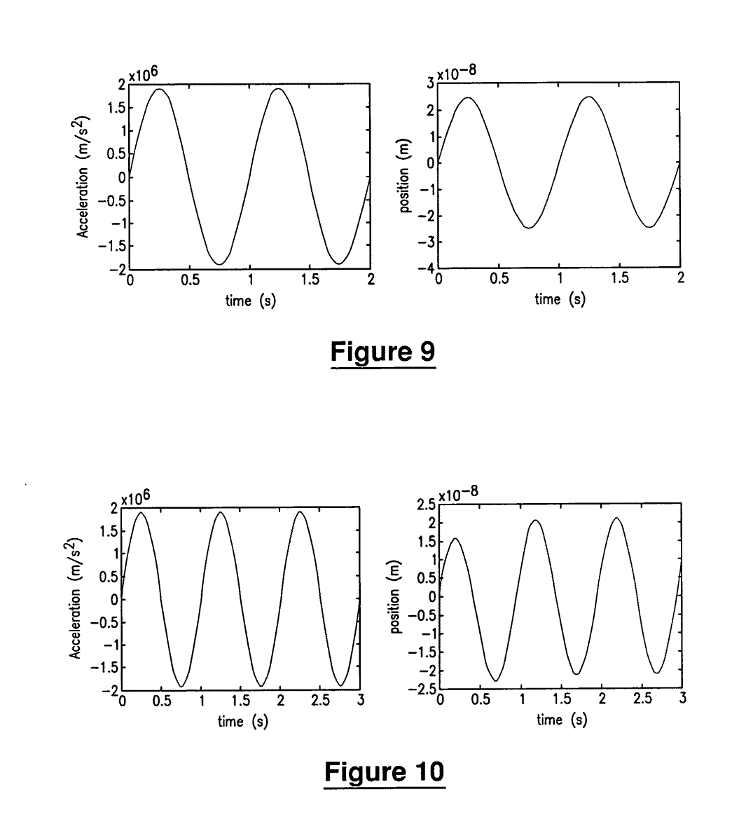

Using Equation (21) with the material and beam properties summarized in Value Set 3 of Table 6, the cyclic acceleration and resulting position data depicted in FIG. 10 were obtained.

TABLE 6
Material and Beam Properties Used to
Calculate the Acceleration Required

| Property | Value Set 3 | |
|---|---|---|
| Material | Gold | |
| E | 79 × 10⁹ | Pa |
| P | 19300 | kg/m3 |
| L | 500 nm-20 μm | |
| H | 10 | nm |
| W | 30 | nm |
| Δ | 20 | nm |
| Proofmass | 0 | kg |

| | |
|---|---|
| Ad | 2.5 × 10⁻⁸ |
| Ωd | 2π |
| R | 5 × 10⁻⁴ |

The damped cantilever reaches the same deflection in 1.15 seconds.

II.F.3. Comparison

In order to compare the different models, the acceleration required to deflect a 1 μm long cantilever with no additional proof mass was calculated. All deflections were 20 nm. There were two undamped models, one based on static acceleration and the other on harmonic acceleration, and one undamped model using a harmonic acceleration. This data is summarized in Table 7.

TABLE 7
Data Obtained from the Various Models Using the Same Parameters

| Method | Acceleration (m/s²) |
|---|---|
| Static Acceleration | 5.5 × 10⁶ |
| Driven Harmonic Oscillator | 1.6 × 10⁶ |
| Damped Driven Harmonic Oscillator | 1.6 × 10⁶ |

The results from the two undamped models were similar within an order of magnitude. The acceleration for a cantilever with damping was the same as the same cantilever without damping. The difference was in the time it takes the cantilever to reach the desired amount of deflection. The undamped cantilever reached 20 nm of deflection in 0.15 seconds, while the damped cantilever reached the same deflection in 1.15 seconds. This difference can be taken into account in the device design.

II.G. Device Design

In some embodiments, the device includes an array of cantilevers that maximizes the amount of resolution attainable while minimizing the overall device size. The fabrication of the device can also be considered when designing the device. In some embodiments, the array measures 1-10 g logarithmically. This particular range is relevant to many applications and can be easily tested in a laboratory setting. The array can also measure less than 6-8 μm in any dimension.

The array can include any number of nanocatilevers. In some embodiments, the array includes 1, 5, 10, 20, 30, 40, 50, 60, 70, 80, 90, 100, or any whole number between 1 and 100 inclusive nanocantilevers that are each 30 nm wide with a pitch of 50 nm. The smallest pitch possible is desirable in order to maximize the number of cantilevers in a given amount of space. In some embodiments, the cantilevers are 10 nm thick. If the cantilevers are designed without a proof mass, which simplifies the fabrication process significantly, the cantilevers can vary in length from 15.2 μm to more than 25 μm, which exceeds the stiction critical length of 21.3 μm and also significantly exceeds the desired size of 6-8 μm. Therefore, in such embodiments, a proof mass can be employed.

The addition of a proof mass to each cantilever allows the cantilevers to be shorter in length. However, the tradeoff between length of cantilevers and the size of the proof mass should be carefully considered. As the proof mass increases in mass, the length of the cantilevers can be substantially reduced but the dimensions of the proof mass also increase significantly. The proof mass dimensions can be constrained by the width of the cantilever and in some embodiments, are as short in length as possible in order to avoid adding additional stiffness to the cantilever. The proof mass can be made from virtually any material, but for ease of fabrication, the same material as the cantilever can be used.

Table 8 summarizes potential proof mass sizes, cantilever lengths, and proof mass dimensions. The proof mass dimensions given are for the cantilever of the shortest length in the range provided.

TABLE 8
Potential Proof Mass Sizes Along with Dimensions that Could be
Added to Each Cantilever and the Resulting Cantilever Lengths^{b}

| Proof | Cantilever | Proof Mass Dimensions | |
|---|---|---|---|
| Mass | Lengths (l) | (w × hₘ × lₘ) | |
| (kg) | (nm) | (nm) | |
| 1 × 10⁻¹² | 500-1050 | lₘ = 0.1(l) | 10 × 3.5E7 × 50 |
| | | lₘ = 0.25(l) | 10 × 1.4E7 × 125 |
| | | lₘ = 0.33(l) | 10 × 1.0E7 × 165 |
| | | lₘ = 0.5(l) | 10 × 6.9E6 × 250 |
| 1 × 10⁻¹³ | 1050-2250 | lₘ = 0.1(l) | 10 × 1.6E6 × 105 |
| | | lₘ = 0.25(l) | 10 × 6.6E5 × 263 |
| | | lₘ = 0.33(l) | 10 × 5.0E5 × 247 |
| | | lₘ = 0.5(l) | 10 × 3.3E5 × 525 |
| 1 × 10⁻¹⁴ | 2250-4900 | lₘ = 0.1(l) | 10 × 76,761 × 225 |
| | | lₘ = 0.25(l) | 10 × 30,704 × 563 |
| | | lₘ = 0.33(l) | 10 × 23,261 × 743 |
| | | lₘ = 0.5(l) | 10 × 15,352 × 1250 |
| 1 × 10⁻¹⁵ | 4900-10,500 | lₘ = 0.1(l) | 10 × 3525 × 490 |
| | | lₘ = 0.25(l) | 10 × 1410 × 1225 |
| | | lₘ = 0.33(l) | 10 × 1068 × 1617 |
| | | lₘ = 0.5(l) | 10 × 705 × 2450 |
| 1 × 10⁻¹⁶ | 10,500-20,000 | lₘ = 0.1(l) | 10 × 164 × 1050 |
| | | lₘ = 0.25(l) | 10 × 66 × 2625 |
| | | lₘ = 0.33(l) | 10 × 50 × 3465 |
| | | lₘ = 0.5(l) | 10 × 33 × 5250 |

^{b}The proof mass dimensions given are for the cantilever of the shortest length in the range provided

The addition of a 1×10⁻¹² kg proof mass gives the most desirable cantilever lengths, but the proof mass dimensions are infeasible. To satisfy the desired maximum package size of 6-8 μm, a proof mass of up to 1×10⁻¹⁴ kg would have to be added to each cantilever. However, the dimensions for a proof mass of that size might not be feasible. Adding a proof mass between 1×10⁻¹⁵ and 1×10⁻¹⁶ kg might surpass the desired maximum device size, but the cantilever lengths would be less than the stiction critical length and the proof mass dimensions are relatively feasible. Selecting a 1×10⁻¹⁵ kg proof mass results in an array that ranges in length 5-11 μm which might not successfully meet the target dimensions.

II.H. Fabrication Method

Considering the fabrication methods commonly used in literature to fabricate nanocantilevers, in some embodiments, using a combination of electron beam lithography, nanoimprint lithography, and release techniques can be employed. The pitch and size of the cantilever array can be very aggressive and might require the use a high resolution patterning technique. In some embodiments, electron beam lithography can be employed to make the first model and can subsequently be used to make the mold used in nanoimprint lithography in order to make many arrays for testing. After these processes, the cantilevers would need to be released and in some embodiments, critical point drying can be used to ensure that stiction did not occur during the fabrication process.

II.I. Measurement Methods

In addition, a method to measure the cantilever deflection prior to integration with the chemistry is required. An exemplary method can be to use a laser to measure the acceleration as the cantilever array spins on a centrifuge. This option can require careful calibration of the laser and centrifuge timing. Another option can be to attach a biological molecule like streptavidin to the substrate and biotin to the cantilever. When streptavidin and biotin come into contact upon cantilever deflection, they can bind to each other. A limitation to this technique can be that once a cantilever has deflected, it cannot return to its initial state without unbinding the two molecules.

II.J. Alternative Approaches

Instead of using an array of nanocantilevers to sense the acceleration, several potential alternative approaches, including nanochannels and buoyancy-driven flow, can be employed.

II.J.1. Nanochannels

Nanochannels are currently used in research to model biological processes on a nanoscale, such as cell sorting, DNA analysis, and in the study of biological components such as DNA and cells (Tegenfeldt et al., 2004). At the nanoscale, the flow is laminar and driven by viscous forces instead of inertial forces. Fluids are typically driven through nanochannels using a pressure difference and/or electrokinetically.

To sense acceleration using nanochannels, accelerations can cause fluid to move through the channels and by measuring some quantifiable difference in concentration or mass flow rate of particle species present in the fluid flow, the acceleration magnitude and duration can be identified and recorded. Four major factors that can contribute to the fluid flow are the acceleration of the channel, diffusion, a difference in pressure between the ends of the channel, and charged channel walls. The mass transfer of ionic species in a nanochannel with charged walls has been modeled and can be used to develop a model of all the contributing factors to the fluid flow (see Conlisk et al., 2002).

II.J.2 Buoyancy-Driven Convection—Acceleration

Buoyancy-driven flow is another possible alternative approach. This concept was recently used to develop a polymerase chain reaction (PCR) device that used a micro-“race track” to heat and cool the fluid (Ness et al., 2004). PCR requires cycling temperatures to denature and anneal the DNA strands in order to amplify the strands. Using different temperatures at different areas of the track, the DNA segment is amplified as it travels around the track.

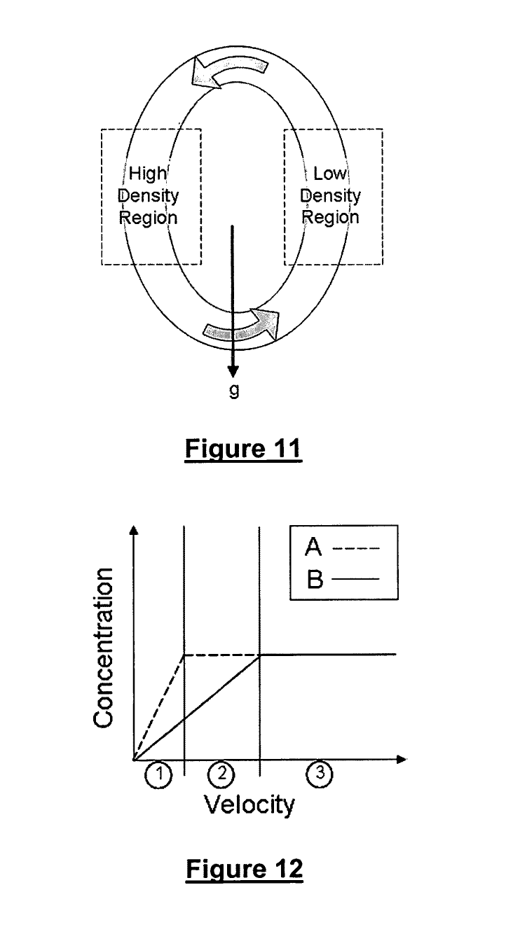

A similar configuration could be used to measure acceleration. One region of the track would contain a high density of chemical units and the opposite region would contain a low density of chemical units (FIG. 11). Essentially the high density region is a source, so it could be that an inlet would be located that would serve as the addition point for the specific chemical units. Another option is to have the walls contain the desired chemical unit. The low density region is a sink where the chemical units are consumed, for example, through binding. Therefore, the concentration increases through the high density region and decreases through the low density region. A lower velocity results in a lower concentration.

Multiple units in different concentrations can be present in the high density region. For instance, if A and B are present on the wall and A is in greater concentration than B, A will saturate before B. Sample data is shown in FIG. 12. As the velocity increases, the concentration of A and B increase at different rates which results in three regions. In Region 1, A and B are increasing with increasing velocity but at different rates and by evaluating the ratio of A to B, the velocity can be determined. In Region 2, A has saturated and B is still increasing, and by evaluating the level of B, the velocity can be determined. In Region 3, both A and B have saturated and the maximum recordable velocity has been reached.

As an initial approximation, mass convection can be used to calculate the length of the channel that the fluid carrying a mass species will move under a specific acceleration. Using

$$Ra = Gr \cdot Sc = \frac{a ( \frac{Δ ρ}{ρ} ) {L}^{3}}{{v}^{2}} \cdot \frac{v}{{D}_{AB}} , \qquad ( 24 )$$

where Ra is the Rayleigh number, Gr is the Grashof number, and Sc is the Schmidt number, which are all dimensionless numbers. The Grashof number relates buoyancy to viscosity (v) and the Schmidt number relates viscosity to mass diffusivity, D_{AB}. Between the range

1708<Ra<5×10⁴,

convection will occur. A length of 23 μm is obtained, using Ra=2000, a=10 m/s², water as the fluid, and the diffusivity of short DNA segments, D_{AB}=6.12×10⁻¹¹ m²/s. At a=100 m/s², the length decreases to 11 μm. For comparison, using sugar as the particle, which has a D_{AB} value of 6.9×10⁻¹⁰ m²/s, the length is 52 μm at a=10 m/s². These lengths are reasonable, although they do not fall into the desired package size of 6-8 μm.

Buoyancy-driven flow can also be the result of a density gradient which is achieved through different temperatures or concentrations. Fluids at higher temperatures often have lower densities compared to fluids at lower temperatures. In the regions of higher temperature, the fluid will rise and in the regions of lower temperature, the fluid will sink, creating a circulating flow. Similarly, regions containing different concentrations also create a circulating flow.

Using this phenomenon, the presently disclosed subject matter can in some embodiments, comprise a micro-fabricated track and the mechanical sensing of the acceleration is accomplished by variations in fluid flow. By establishing regions of different temperatures and/or concentrations of chemical species, different accelerations can be recorded. Different regions of temperature can be achieved by using materials with different conductivities to construct the track, developing heat sinks, and heating specific areas of the track. For different regions of concentration, one region of the track acts as a source and chemical species are added either through an inlet or through pre-existing concentrations on the walls. Another region of the track acts as a sink either through binding of the chemical reactants or through an outlet.

II.J.2.a. Heat Transfer

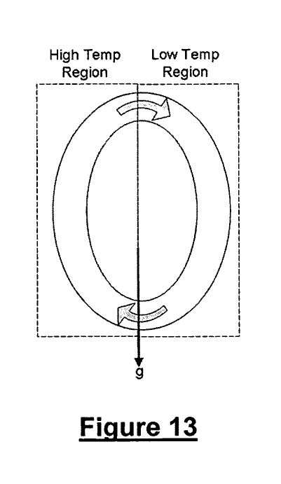

In some embodiments, a track can be divided into a high temperature region and a low temperature region, depicted in FIG. 13. Due to the density gradient and presence of gravity, a fluid flow is created.

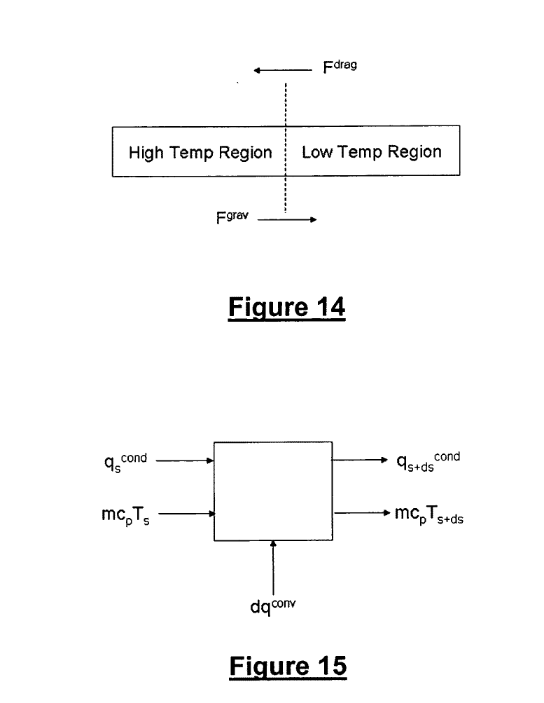

To model the system, the conservation of momentum is applied. The circular track is modeled as a 1-D channel with repeating boundary conditions, shown in FIG. 14. The forces on the system include a gravitational force and a frictional drag force. The gravitational force over the length of the channel, L, is

$${F}^{grav} = {\int }_{0}^{L} ρ {A}_{c} {g}_{s} d s \qquad ( 25 )$$

where ρ is the density of the fluid, A_{c} is the cross-sectional area of the channel, and gₛ is defined as

$${g}_{s} = g sin ( 2 π \frac{s}{L} ) . \qquad ( 26 )$$

The drag force over the length of the channel is

$${F}^{drag} = {\int }_{0}^{L} \frac{1}{2} {C}_{D} ρ {u}^{2} ( π {D}_{H} ) d s \qquad ( 27 )$$

where u is the fluid velocity, DH is the hydraulic diameter, and C_{D} is the drag coefficient defined as

$${C}_{D} = \frac{16 μ}{ρ {uD}_{H}} \qquad ( 28 )$$

where μ is the viscosity of the fluid. This substitution results in the following equation for the drag force

$${F}^{drag} = {\int }_{0}^{L} 8 μ u π d s . \qquad ( 29 )$$

The conservation of momentum is

$${\int }_{0}^{L} ρ {A}_{c} {g}_{s} d s - {\int }_{0}^{L} 8 πμ u d s = \overline{ρ} {A}_{c} L \frac{\partial u}{\partial t} . \qquad ( 30 )$$

The following substitutions are made

ρ=ρ^{o}+ρ′

u=u^{o}+u′ (31)

where ρ^{o} and u^{o} are constants and represent a biased background value for density and fluid velocity respectively and ρ′ and u′ vary in time and space. With these substitutions, the momentum equation becomes

$${\int }_{0}^{L} \frac{{ρ}^{′}}{{ρ}^{o}} {g}_{s} \frac{d s}{L} - \frac{8 πμ}{\overline{ρ} {A}_{c}} {u}^{o} = \frac{\partial {u}^{′}}{\partial t} . \qquad ( 32 )$$

In order to solve this partial differential equation, Fourier series expansions are defined as

$$\frac{{ρ}^{′}}{{ρ}^{o}} = {\sum }_{n} [ {a}_{n} cos ( \frac{2 π n s}{L} ) + {b}_{n} sin ( \frac{2 π n s}{L} ) ] \\ {u}^{′} = {\sum }_{n} [ {A}_{n} cos ( \frac{2 π n s}{L} ) + {B}_{n} sin ( \frac{2 π n s}{L} ) ] \qquad ( 31 )$$

Finally, a basis function is used to solve the equation which resulted in two ordinary differential equations

$$\frac{{b}_{n} g}{2} - \frac{8 πμ}{\overline{ρ} {A}_{c}} {u}^{o} = \frac{L}{2} \frac{\partial {A}_{n}}{\partial t} \\ \frac{{b}_{n} g}{2} - \frac{8 πμ}{\overline{ρ} {A}_{c}} {u}^{o} = \frac{L}{2} \frac{\partial {B}_{n}}{\partial t} \qquad ( 32 )$$

Next, the conservation of energy is applied. The energy balance is

Ėᵢₙ−Ėₒᵤₜ+Ė_{gen}=Ėₛₜ (33)

and is depicted in FIG. 15. After substituting for the different energy terms, the energy balance becomes

$$k {A}_{c} \frac{\partial }{\partial s} ( \frac{\partial T}{\partial s} ) ds + h d {A}_{wall} ( T - {T}_{wall} ) + \dot{m} {c}_{p} \frac{\partial T}{\partial s} ds = {c}_{p} {A}_{c} \frac{\partial T}{\partial t} ds \qquad ( 34 )$$

where T is the temperature, k is the thermal conductivity, h is the heat transfer coefficient, \dot{m} the mass flow rate, cₚ is the specific heat of the fluid. The following substitutions are made

$$d {A}_{wall} = π {D}_{H} ds \\ h = \frac{k}{L} N u = 3.66 \frac{k}{L} \\ α = \frac{{c}_{p}}{k} \qquad ( 35 )$$

which results in the energy equation

$$\frac{\partial }{\partial s} ( \frac{\partial T}{\partial s} ) + \frac{3.66 π {D}_{H}}{{A}_{c} L} ( T - {T}_{wall} ) + \frac{\dot{m} {c}_{p}}{k {A}_{c}} \frac{\partial T}{\partial s} = α \frac{\partial T}{\partial t} \qquad ( 36 )$$

Similar to the substitutions made in the conservation of momentum, the following substitutions are made

T=T^{o}+T′

m=m^{o}+m′ (37)

which results in the following equation

$$\frac{\partial }{\partial s} ( \frac{\partial {T}^{′}}{\partial s} ) + \frac{α {\dot{m}}^{o}}{{A}_{c}} \frac{\partial {T}^{′}}{\partial s} + \frac{3.66 π {D}_{H}}{{A}_{c} L} ( {T}^{o} + {T}^{′} - {T}_{wall} ) = α \frac{\partial {T}^{′}}{\partial t} . \qquad ( 38 )$$

The following Fourier series expansions are used to solve the partial differential equation

$${T}^{′} = {\sum }_{n} [ {c}_{n} cos ( \frac{2 π n s}{L} ) + {d}_{n} sin ( \frac{2 π n s}{L} ) ] \\ {T}_{wall} = \frac{{T}_{H} + {T}_{C}}{2} + \frac{4 ( {T}_{H} - {T}_{C} )}{2 π} {\sum }_{n = 1 , 3 , 5 …}^{∞} \frac{1}{n} sin ( \frac{2 π n s}{L} ) . \qquad ( 39 )$$

Solving the energy equation with basis functions results in the following ordinary differential equations

$$\frac{- 2 {π}^{2} {c}_{n}}{L} + \frac{{d}_{n} π α {\dot{m}}^{o}}{{A}_{c}} + \frac{3.66 π {D}_{H}}{{A}_{c} L} ( {T}^{o} + \frac{{c}_{n} L}{2} - \frac{{T}_{H} + {T}_{C}}{2} + \frac{4 ( {T}_{H} - {T}_{C} ) L}{3 {π}^{2}} ) = \frac{α L}{2} \frac{\partial {c}_{n}}{\partial t} \\ \frac{- 2 {π}^{2} {d}_{n}}{L} - \frac{{c}_{n} π α {\dot{m}}^{o}}{{A}_{c}} + \frac{3.66 π {D}_{H}}{{A}_{c} L} ( {T}^{o} + \frac{{d}_{n} L}{2} - \frac{{T}_{H} + {T}_{C}}{2} ) = \frac{α L}{2} \frac{\partial {d}_{n}}{\partial t} . \qquad ( 40 )$$

The system of equations for the heat transfer model is

$$[ \begin{matrix}{\dot{A}}_{n} \\ {\dot{B}}_{n} \\ {\dot{c}}_{n} \\ {\dot{d}}_{n}\end{matrix} ] = [ \begin{matrix}0 & 0 & 0 & 0 \\ 0 & 0 & 0 & 0 \\ 0 & 0 & - \frac{4 {π}^{2}}{α {L}^{2}} + \frac{3.66 π {D}_{H}}{α {A}_{c}} & \frac{2 π {m}^{o}}{{A}_{c} L} \\ 0 & 0 & - \frac{4 {π}^{2}}{α {L}^{2}} + \frac{3.66 π {D}_{H}}{α {A}_{c}} & - \frac{2 π {m}^{o}}{{A}_{c} L}\end{matrix} ] [ \begin{matrix}{A}_{n} \\ {B}_{n} \\ {c}_{n} \\ {d}_{n}\end{matrix} ] + [ \begin{matrix}\frac{{b}_{n} g}{L} + \frac{16 π μ {u}^{o}}{{ρ}^{o} {A}_{c} L} \\ \frac{{b}_{n} g}{L} + \frac{16 π μ {u}^{o}}{{ρ}^{o} {A}_{c} L} \\ \frac{3.66 π {D}_{H}}{α {A}_{c} {L}^{2}} ( 2 {T}^{o} - {T}_{H} - {T}_{C} + \frac{8 ( {T}_{H} - {T}_{C} ) L}{3 {π}^{2}} ) \\ \frac{3.66 π {D}_{H}}{α {A}_{c} {L}^{2}} ( 2 {T}^{o} - {T}_{H} - {T}_{C} + \frac{8 ( {T}_{H} - {T}_{C} ) L}{3 {π}^{2}} )\end{matrix} ] \qquad ( 41 )$$

The last additional relationship required is the thermal expansion coefficient, β, which is equal to

$$β = - \frac{1}{ρ} ( \frac{\partial ρ}{\partial T} ) . \qquad ( 42 )$$

After rearranging the equation, applying the same Fourier series previously defined, and solving with the basis function, the following two equations are obtained

βcₙ+aₙ=0

βdₙ+bₙ=0 (43)

The transfer function of the system is

$$G ( s ) = \frac{3.66 {D}_{H} {d}_{n}^{2} β}{α {A}_{c} {A}_{n} s + \frac{α 16 π μ {u}^{o}}{{ρ}^{o} L}} \qquad ( 44 )$$

which can be used to calculate the system bandwidth and gain.

### III. Chemical Recording

III.A. Generally

The mechanical or chemical characteristics of the device determine the instantaneous deflection, deformation, or other physical or chemical change of a valve or some other regulatory mechanism, which provides information about the amount of acceleration or some other relevant change in environmental state variable applied at that particular time. To make this deflection useful, a reporting and recording of the deflection is necessary. However, on the nano size scale, electronics currently cannot be fabricated without great difficulty and expense. Therefore, in addition to the mechanical characteristics of the presently disclosed devices, a chemical component that acts as a mechanism of documenting the deflection is discussed herein. Consistent with these embodiments, a measurable, a quantifiable chemical change can occur that can record the acceleration or other relevant change in environmental state variable and provide some measure of time as each valve deflects. Using the data generated, the time history of the position, in the case of an acceleration or position sensor, or the level of some other relevant and sensed environmental state variable can be calculated.

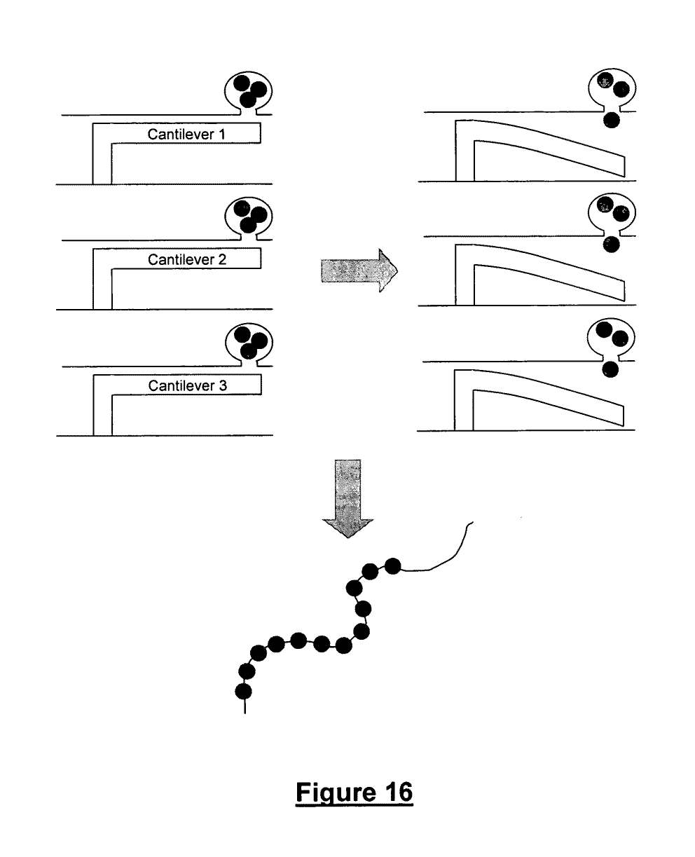

FIG. 16 illustrates the basic concept. In some embodiments, each of the acceleration amplitudes, or threshold level of some other relevant environmental state variable, is assigned to a different chemical unit. For each time unit during which a particular acceleration or other relevant change in environmental state variable is experienced, a discrete number (e.g., one) of chemical units is released. Therefore, the presence of a certain quantity of chemical units can be interpreted to correspond to an amount of time that a particular acceleration or other relevant change in environmental state variable was experienced. As the device goes through a sequence of accelerations or other changes in environmental state variable over time, the chemical units are bound together and the time history of the sensed environmental state variable, in this instance the path taken by the device, can be deduced by analyzing the chain of units and calculating how long each of the accelerations was experienced. A particular chemical unit can be assigned to each of the reservoirs or valves, and can be released as the valves deflect. Potential chemical units include, but are not limited to sugars, amino acids, and nucleotides, either singly or polymers thereof, and combinations thereof. In some embodiments, the units are not exclusively chemical units. They might be, for example, small polystyrene particles that link to each other electrostatically or small magnetic particles that link to each other magnetically; in which case the chains are not necessarily chemically bound. They can also be changes in some detectable property of the chemical unit including, but not limited to color, hardness, stiffness, shape, temperature, or fluorescence. Furthermore, the mechanism for storing the chemical units prior to their release for incorporation into a polymer, referred to here as storage in a “reservoir,” can involve storage by mechanisms other than containment in a physical volume. In some embodiments, the chemical units can be chemically, magnetically, or electrostatically bound to specific sites on a surface, stored within the folds of a conformationally-changing protein, freely floating in the reaction chamber but with a conformation, or otherwise made inaccessible for inclusion into a polymer.

Furthermore, changes in any of a variety of detectable properties, referred to herein as “environmental state variables”, can be recorded. In some embodiments, sensed environmental variables other than acceleration can be detected and recorded. These include, but are not limited to, changes in temperature, pressure, concentration, pH, intensity of incident light or other electromagnetic radiation, frequency of incident light, sound intensity, sound frequency, binding events of antigens or other molecules to a binding site, velocity, strength of magnetic field, and electric field strength. Furthermore a variety of mechanisms for the governing of the release of chemical units from the reservoirs, referred to herein as “valves” or “valve mechanisms”, can be used. The valve mechanism illustrated in the text above is a cantilever, but other valve mechanisms can be used. In some embodiments, potential valve mechanisms include, but are not limited to: cantilever structures; variable porosity membranes such as those of thermosensitive liposomes or other types of liposomes, porous protein complexes, or other biomolecular containers; pores, perforations, or hatches in shells made of plastics, ceramics, metals, or other non-biological materials; and conformation changes of proteins, protein complexes, or other structures with multiple stable conformations.

III.B. Unit Selection

There are many embodiments for the chemical units assigned to each of the acceleration magnitudes or sensed levels of other relevant environmental state variables, including but not limited to sugars, amino acids, nucleotides, either singly or polymers thereof, and combinations thereof, and changes in a variety of properties such as, but not limited to, fluorescence, concentration, and pH. For the techniques described herein, the chemical units selected are nucleic acids. This selection was done for a number of reasons. First, unique sequences can be designed for each valve that can be later used in the data analysis to identify the corresponding change in sensed environmental state variable. Secondly, these sequences can bind without being affected by sterics. Finally, the double helix of DNA that results from polymerization of individual nucleic acids is compact in size, approximately 2-3 nm in diameter and 3-4 nm long for a full turn of the helix, which corresponds to between 11 and 12 base pairs.

III.C. Unit Storage

Before the chemical units are released by deflection of a valve, they can be stored in a reservoir. Under the valve approach outlined herein above, each valve can have its own respective reservoir. There are at least two primary options for how the units could be stored in these reservoirs. They could be stored as individual units that float throughout the reservoirs. To prevent the units from binding together in the storage reservoirs, environmental conditions can be maintained so that in the reservoir the units remain unbound. In the mixing chamber, however, the environmental conditions can be optimized for binding, polymerization, etc. Another option is to have individual units stored in a strand or other polymer that can be cut at predetermined points along the polymer during the deflection of the valve using, for example, a nuclease.

III.D. Data Analysis

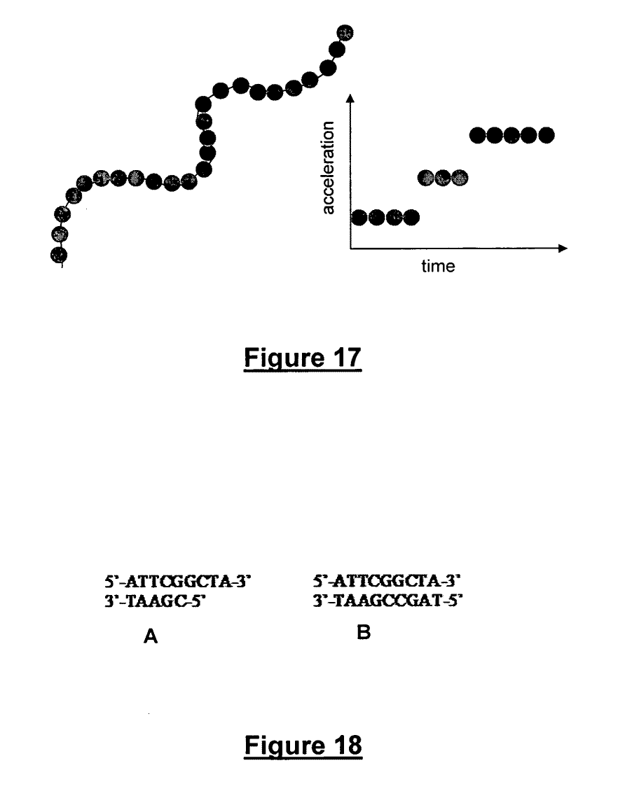

FIG. 16 shows a simplified strand of data resulting from a series of accelerations and valve deflections. In some embodiments, each of the valves in the array is designed to deflect a specified amount when it experiences a particular acceleration or a change in some other relevant environmental state variable. However, at such an acceleration or change, the assigned valve as well as any other valves that corresponds to a lower acceleration will deflect. For example, assigning “Cantilever 1” in FIG. 16 to the cantilever sensing the lowest acceleration and each subsequent cantilever to an increasing acceleration, when Cantilever 2 deflects, Cantilever 1 also deflects. Therefore, the data strand (i.e., the polymer of nucleotides, sugars, amino acids, etc.) might be expected to appear more like the strand shown in FIG. 17. The units released for each of the accelerations bind either randomly or in a predetermined order that can be selected during sequence design.

This potential result signifies that data analysis subsequent to any “run” can require taking into account the potential for multiple units to be released by greater accelerations or changes in some other relevant environmental state variable. In some embodiments, data analysis is simplified by including a starting sequence onto which all subsequent units bind in the chamber or area where the individual units flow to interact and ultimately be measured. This starting sequence, also called herein a primer, can be designed in such a way as to allow binding in only one direction, thus preventing the units from binding randomly on either side which can complicate the data analysis. Preferably, the units are designed to bind sequentially and the data is analyzed by looking for the unit corresponding to the highest acceleration in each section, cancelling out an equal number of lower accelerations, and determining the amount of time the particular acceleration was experienced iteratively down the length of the polymer. Furthermore, in some embodiments, this starting sequence can be, but is not required to be, bound to a surface to prevent it moving freely through the reaction chamber.

It should be noted that it is possible that “subchains” can form. If chemical units come into contact with each other before reaching the main chain, then it can be possible for them to bind to each other prematurely, thereby forming an aberrant subchain. However, even in the presence of subchains, the chains could be evaluated in a similar matter as described because there should be a probabilistic distribution of lengths and unit content.

III.E. Fluorescence

In some embodiments, fluorescence is employed for an instantaneous reporting of a state. Each of the reservoirs or valves can be equated with a given fluorescence which is released as an acceleration, temperature change, or other relevant change in environmental state variable is experienced. A particular fluorophore can be associated with DNA binding so that the detectable fluorescence is altered as units are bound and/or to particular sequences so that the detectable fluorescence is altered in the mixing chamber as more chemical units are added. This reporting scheme can provide an ability to report an overall state of the system at a particular timepoint, but need not provide any inherent recording of the history. In some embodiments, this reporting scheme may also be used to read information already recorded into a polymer strand.

III.F. Description of Exemplary Techniques

The following sections outline potential chemical techniques that can be used to accomplish this measurement. It is understood that the following sections are not intended to represent an exhaustive list of techniques that can be employed with the presently disclosed subject matter.

III.F.1. Chemical Unit Release

As previously described, in some embodiments each valve of the array has a specific chemical unit assigned to it. Upon the deflection of a given valve, one or more of the units assigned thereto (in some embodiments, one) is released. Preferably, the units that are released by different valves bind to each other sufficiently quickly and efficiently to provide an accurate time history and order that can be used to decipher the sequence and time lengths of valve deflections. This recording technique can thus depend on diffusion and binding times and the ability to extract the string of units in order to analyze the sequence.

Furthermore, as previously mentioned, a variety of mechanisms may be used to accomplish and govern the release of chemical units from the reservoirs in which they may be stored. Collectively, these various mechanisms are referred to herein as “valves” or “valve mechanisms.” In some embodiments, the valve mechanisms may be, but are not limited to be: cantilever structures; variable porosity membranes such as those of thermosensitive liposomes or other types of liposomes, porous protein complexes, or other biomolecular containers; pores, perforations, or hatches in shells made of plastics, ceramics, metals, or other nonbiological materials; and conformation changes of proteins, protein complexes, or other structures with multiple stable conformations. Also, herein the phrase “deflect” refers to any change in a “valve” or “valve mechanism” which causes a change in the rate at which chemical units are permitted to pass through the valve mechanism.

III.F.2. Predetermined DNA Path

In some embodiments, a predetermined DNA path is employed to record a series of accelerations or the time history of some other relevant environmental variable. The path can be composed of a single strand of DNA and can detail a specific progression of accelerations or changes in other relevant environmental variables and times. Each of the valves can be assigned to its own unique single stranded DNA sequence either of the same or varying base pair length. As a specific valve is deflected at a given acceleration, it releases its assigned DNA sequence at a given known time rate. These sequences can be the complement of different portions of the predetermined path and can thus bind to specific area along the path.

While this method is among the easiest techniques to execute and analyze, using a predetermined path as a basis for recording data from the operation of the presently disclosed subject matter can limit its application. Particularly, only knowledge of progression on that specific path would be provided. No variations of the path could be recorded. For example, if the predetermined path was ABBBCDDEEEE, where each letter corresponds to a valve, its respective DNA sequence, and one time unit, and the device actually completed AAABCCC, only the following data would be recorded:

| | |
|---|---|
| Actual Path: | A A A B C C C |
| Predetermined Path: | A B B B C D D E E E E |
| Data Recorded: | A B C |

which is an incomplete and erroneous record of what was actually experienced by the system. The applicability of this recording technique thus can depend on the accuracy and reliability of the DNA sequences binding with their complement on the predetermined path, and can be limited to those applications that only need to determine whether a certain predetermined path has been completed. It also depends on the ability to separate and extract the predetermined path along with whatever sequence complements have bound from all of the extraneous sequences.

III.F.3. DNA Computing

In order to provide more flexibility over a single predetermined DNA path, the principles of DNA computing can be employed. DNA computing was proposed in 1994 by Adleman who solved a Hamiltonian path problem consisting of seven points (Adleman, 1994). Similarly, predetermined paths can be generated for many paths or maybe even every possible path of a certain length. The sequences can be released and bind to the proper complements. Ligase can be released at the end of the device's operation to ligate the segment complements into a complete unbroken single strand. Other enzymes can also be employed depending on the chemical nature of the recording unit. For example, terminal deoxynucleotidyl transferase (TdTase) can also be employed for polymerizing nucleotides as set forth in more detail hereinbelow.

The feasibility of this technique can depend on the accuracy and reliability of the DNA sequences binding with their complement on the predetermined path and the ability to separate and extract the predetermined path along with whatever sequence compliments have bound from all of the extraneous sequences. This former dependency can be important since for each time unit a given deflection releases a particular sequence and this sequence can bind to its complement on any of the given number of paths. The next time unit, a given deflection also releases a particular sequence, but this sequence would not necessarily bind to the same path that the previous sequence had. Therefore, the path data would be scattered among all the different possible paths and the time data could be lost. This scattering can be accounted for by increasing the release rate of sequences, which can increase the probability of a sequence binding with previous sequences.

III.F.4. DNA Sticky Ends

A DNA sticky end is a single stranded overhang that extends from a double strand of DNA (see FIG. 18A). In some embodiments, each valve can have a specific DNA sequence with a sticky end on one or both sides, which would represent a given acceleration. As a device underwent a series of accelerations or changes in other relevant environmental state variables, the corresponding valves deflect and the analogous sequences are released. As long as the sticky ends are complementary, the sequences would bind to each other via the overhangs. The feasibility of this embodiment can depend on several things including the reliability and efficiency of the DNA binding between complementary sticky ends, the ability to extract out the data, and the sequence design of the overhangs.

The sequence design can be particularly important. If sequences are designed so that sequence A can only bind to sequence B and sequence B can only bind to sequences A and C, etc. when the progression AAABBCCCCD is completed, the resulting strands would look like: ABCD and ABC with the additional sequences not binding to anything. An alternative sequence design is to design all of the sequences to have the same sticky end sequences but to also include a unique sequence within the double stranded region that serves as the identifier of the specific reservoir or valve to which the sequence was assigned. In these embodiments, all of the sequences can potentially concatenate with each other with equal probability. The identifying sequence within the duplex region can be in some embodiments, at least four base pairs in length (e.g., 4, 5, 6, 7, 8, 9, 10, 11, 12, or more base pairs in length) to ensure sufficient sequence variety. Additionally, the sticky ends can be at least 4, 5, 6, 7, 8, 9, 10, 11, 12, or more nucleotides in length to ensure that stable binding occurs between the sticky ends under the conditions of the run (e.g., at room temperature). In some embodiments, the sticky ends are at least 7 nucleotides in length, which can makes each segment at least eighteen nucleotides long (e.g., at least a four base pair segment with two seven nucleotide sticky ends).

III.F.5. Blunt End Ligation

Blunt end ligation refers to the joining of non-single-stranded (e.g., double-stranded) nucleotide sequences that do not have terminal overhangs (see FIG. 18B). In some embodiments, each valve or reservoir is assigned a unique blunt ended sequence of at least four base pairs. These segments are released upon deflection of the valve or valve mechanism and bind to other segments in the presence of a specific ligase. The efficiency of this option can depend on several things including the reliability and efficiency of binding between the respective blunt ends, the ability to extract out the data, and data analysis.

III.F.6. Single Strand DNA Recording Units

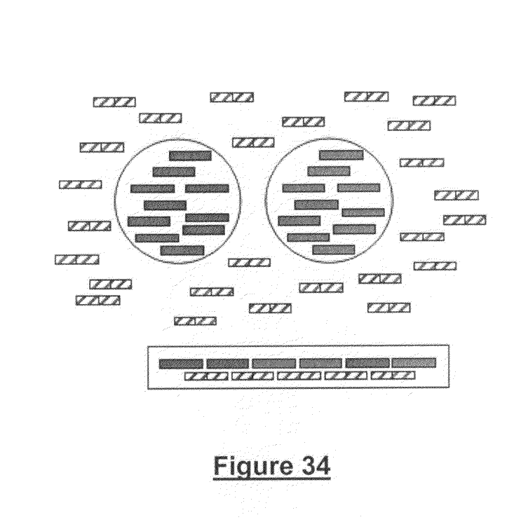

In this concept, shown in FIG. 34, each variety of thermosensitive liposome encapsulates a unique 20 to 24 nucleotide long single strand of DNA. The length of the oligonucleotides was balanced between efforts to make it as short as possible while also ensuring that the melting temperature of the strand would be high enough to withstand the elevated liposome transition temperatures. This was done primarily through the GC content. The sequence design also had to ensure that there were no internal structures that would prevent the sequences from properly binding with each other.

In the surrounding solution, in addition to ligase and cofactors, the complement of each of these DNA strands is present, as well as the linkage complement. The linkage complement takes the last half of one DNA strand complement and the first half of another DNA strand complement. As depicted in FIG. 34, solid blue and red bars denote the unique DNA strands (chemical recording unit) and hashed blue and red bars denote the complements to the solid blue and red bars, respectively. In addition to the exact complement of red and blue, linkage complements must also be available. Thus, if a “red” DNA strand and a “blue” DNA strand are released, a complement link must be present in order to record the order. Upon encountering the threshold temperature the unique DNA strands encapsulated within the liposomes (circles) are released, whereby the DNA strands bind with the corresponding complement to form a single strand of DNA (inset), wherein the position and number of each DNA strand (chemical recording unit) in the single strand of DNA is indicative of a reading of the environmental state variable, e.g. temperature, at a given point in time.

Also present in solution is a start sequence. The start sequence is either 20 or 22 nucleotides long. At one end of the start is a double strand stretch of 10 bases that is the same across all the start sequences. The remainder of the start sequence is half of the complement of one of the DNA strand sequences. The start sequence can be selected to be the complement to any of the DNA strands with the original DNA present in solution.

This DNA scheme uses T4 DNA ligase present in solution. It is also possible to add the ligase to samples removed from the solution during experiments.

III.G. Efficiency Parameters

To determine the efficiency of the different recording techniques discussed herein, several parameters can be considered including, but not limited to binding rates, diffusion times, data analyses, ease of experiments, and capabilities. The binding rates can determine the amount of time required for the DNA segments to bind, and faster binding rates can be desirable. The diffusion times can determine how long it takes a DNA segment to move between two points, for example, from the entrance of a mixing chamber to a binding site. A faster diffusion time can also be desirable. Data analysis can consider the ease in analyzing the resulting data strand after the segments have been released and bound. The ease of the experiment parameter can consider the level of difficulty inherent in running each of the recording techniques proposed in terms of materials and equipment required, experiment sensitivity, and/or reliability. The recording technique's capability describes its ability to record a variety of acceleration scenarios.

III.G.1. Binding of DNA Segments Using T4 DNA Ligase

Enzymatic ligases can be used to bind strands of DNA and/or RNA together under specific conditions either by closing nicks such as those that occur when sticky ends bind or through blunt end ligation (see e.g., Sgaramella et al., 1970). T4 DNA ligase can be obtained from infecting susceptible E. coli with bacteriophage T4 and can be used to bind a 3′ hydroxyl group on one nucleotide strand to a 5′ phosphoryl group on the same or another nucleotide strand in three reaction steps (Lehman, 1974). The first step in the reaction is the activation of the enzyme through the presence of ATP which results in the formation of ligase-adenylate and pyrophosphate (PPi). Next, a pyrophosphate linkage is made between the 5′ end of the DNA to be joined and the adenosine monophosphate (AMP) by the ligase transferring the adenylate group to the DNA. Finally, a phosphodiester bond is formed between the 5′ phosphate end and the 3′ hydroxyl end and the AMP is removed (Weiss et al., 1968; Rossi et al., 1997). The presence of Mg²⁺ aids in DNA ligation (Weiss et al., 1968; Cherepanov & de Vries, 2003). Therefore, for ligation to occur, DNA fragments, T4 DNA ligase, ATP, Mg²⁺, and water should be present in the solution.

Under the same conditions, T4 DNA ligase has approximately twice the activity in binding sticky ends, which requires nick sealing, than it does in ligating blunt ends (Ferretti & Sgaramella, 1981). However, in the additional presence of RNA ligase, the turnover number for the blunt end ligation increases significantly (although nick sealing performance appears unaffected), which results in the ligation of sticky ends and of blunt ends to be approximately equivalent in efficiency (Sugino et al., 1977). The presence of ATP in certain concentrations can reduce the efficiency of both blunt end ligation and the ligation between the sticky ends. In the presence of 5 mM ATP, blunt end ligation is inhibited, and increasing the concentration to 7.5 mM of ATP inhibits the ligation of the sticky ends (Ferretti & Sgaramella, 1981).

III.G.2. Diffusion

As additional DNA sequences are added upon the deflection of a valve or valve mechanism, they diffuse throughout the solution until they come into proximity to a complementary sequence and bind. An estimate of the time, t, at which the sequences diffuse depends on the distance, x, and the diffusion constant, D. This approximation equals

$$t = \frac{1}{2 D} {( \frac{x}{0.67} )}^{2} . \qquad ( 25 )$$

See Weiss, 1996. The diffusion constant of DNA in water has been calculated experimentally and was found to be

D=4.9×10⁻⁶ cm²/s·[bp]^{-0.72} (26),

where bp is the number of base pairs (Lukacs et al., 2000). Because the DNA sequence is ideally constantly growing in base pair length, the diffusion constant should decrease, increasing the amount of time required to diffuse x.

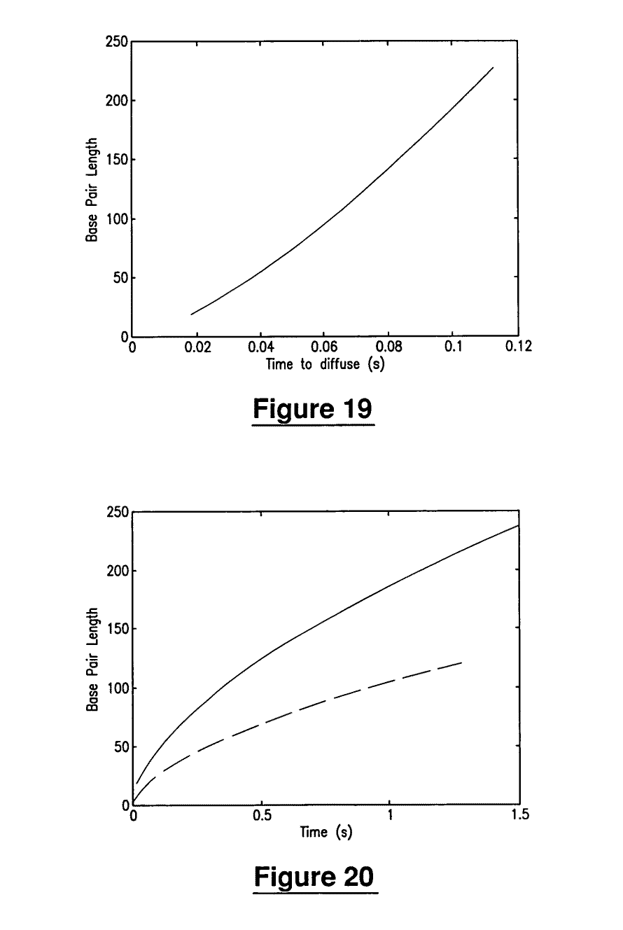

For DNA sticky end embodiments, a sequence that is eighteen base pairs in length, the time to diffuse 1 μm is 0.018 seconds. As an approximation, if the sequence binds to another sequence in the time to diffuse 1 μm, effectively growing eleven base pairs (due to the fact that two of the sticky ends bind together), the diffusion time increases as shown in FIG. 19.

Similarly, for DNA blunt end embodiments, a sequence that is four base pairs in length, the time to diffuse 1 μm is 0.006 seconds. As an approximation, if the sequence binds to another sequence in the time to diffuse 1 μm, effectively growing four base pairs, the diffusion time increases in the same way as shown in FIG. 19 for the sticky end scenario.

FIG. 20 is a graph of the increasing base pair length with the cumulative time, showing that in one second a sticky end sequence embodiment is already 194 base pairs in length, while the blunt end scenario sequence is 104 base pairs in length. In just a minute of operation, under these conditions, the sticky end scenario sequence will be 2053 base pairs long and the blunt end scenario sequence will be 1140 base pairs long.

III.H. Feasibility Comparison

To compare the feasibility of the various recording techniques described, various parameters were considered including the rate of binding of units, diffusion times, data analysis, difficulty of experiment, and capability of the recording technique. A summary of the parameters and techniques is provided in a Pugh Chart shown in Table 9.

TABLE 9
Evaluation of Exemplary Recording Techniques
Using Various Feasibility Parameters

| Recording | Binding | Diffusion | Data | Ease of | |
|---|---|---|---|---|---|
| Technique | Rate | Times | Analysis | Experiment | Capability |
| Chemical | + | +++ | +++ | + | +++ |
| Unit | | | | | |
| Release | | | | | |
| Pre- | +++ | ++ | +++ | +++ | + |
| determined | | | | | |
| DNA Path | | | | | |
| DNA | +++ | ++ | + | ++ | +++ |
| Computing | | | | | |
| DNA Sticky | +++ | ++ | ++ | ++ | +++ |
| Ends | | | | | |
| Blunt End | + | +++ | ++ | ++ | +++ |
| Ligation | | | | | |

Using a qualitative method of comparison, the predetermined DNA path and DNA sticky end methods can be considered the most feasible, but all of the methods are reasonably feasible and can be used as the chemical recording technique. The easiest method to initially test is the predetermined DNA path method.

### IV. Integrated Device Assessment

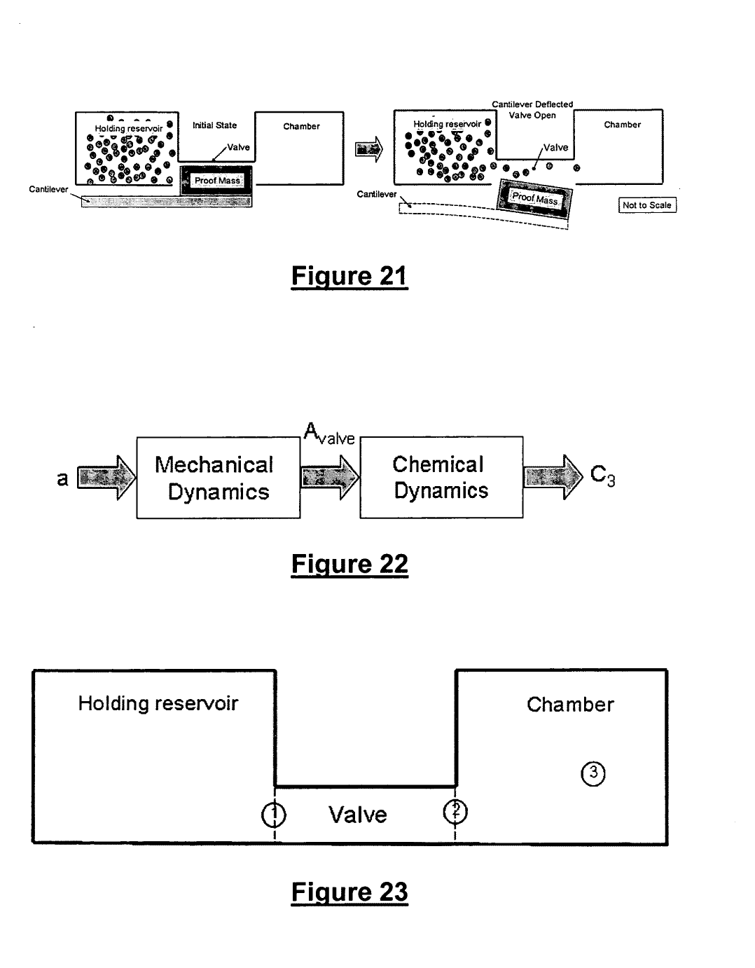

In some embodiments, the devices disclosed herein employ a combination of mechanical sensing and chemical recording. In some embodiments, an array of nanocantilevers has been proposed to sense the acceleration and the binding of unique sticky end sequences as a strategy for recording acceleration. A schematic of the device in operation is shown in FIG. 21. The sticky-end DNA segments are stored in the holding reservoir. When acceleration is experienced, the valve, here a cantilever, deflects and the valve throat opens, which allows the segments to travel into the mixing chamber.

In order to calculate the relationship between the magnitude of the acceleration and the amount of material transferred, transfer functions for the mechanical dynamics and chemical dynamics, depicted as a block diagram in FIG. 22, were developed. Using acceleration, a, as the input, the mechanical dynamics give an output of deflection, x, which can be used to find the cross-sectional area of the valve, Aᵥₐₗᵥₑ. Using Aᵥₐₗᵥₑ as an input to the chemical dynamics, the concentration of the mixing chamber, C3, can be calculated.

The mechanical dynamics are based on the cantilever models previously developed and represent the mechanical sensing of acceleration. The chemical recording comprises the valve transport dynamics, mixing dynamics, and binding dynamics. The valve transport dynamics is the diffusion of the DNA segments through the valve from the holding reservoir to the mixing chamber; the mixing dynamics is the diffusion of the DNA segments through the chamber to the binding site; and the binding dynamics is the consumption of the DNA segments through binding into a single strand of data.

IV.A. Mechanical Dynamics

The mechanical dynamics for the device are in some embodiments, based on a damped cantilever. The transfer function relating acceleration as the input and deflection as the output is

$$\frac{X ( s )}{A ( s )} = \frac{1}{{s}^{2} + 2 {ξω}_{n} s + {ω}_{n}^{2}} . \qquad ( 27 )$$

The gain for the mechanical dynamics is

$$K = \frac{1}{{ω}_{n}^{2}} = \frac{{m}_{eff}}{{k}_{eq}}$$

and the bandwidth is

$$\frac{1}{τ} = 2 {ξω}_{n} .$$

IV.B. Chemical Dynamics

The chemical dynamics were modeled two ways. The first model used a lumped sum approximation and neglected valve dynamics, while the second model took into account a time delay due to the time it would take the particles to travel through the valve. The analysis presented here is cast in terms of an acceleration-sensing embodiment that uses cantilevers as valves. However, it is to be understood that the modeling of the chemical dynamics is generalizable to all mechanisms for the mechanical or chemical sensing of changes in one or more environmental state variables and the chemical recording of the time history of these changes.

IV.B.1. First Approximation

To determine the chemical dynamics and resulting transfer function, a lumped sum approximation was made. This approximation is justified because the Biot number, Bi, is significantly less than 1. Using FIG. 23, the three points of interest are the start of the valve, the end of the valve, and the mixing chamber. The mass flow rates, m, between the different points are

$${\dot{m}}_{1 - 2} = \frac{{MD}_{AB} {A}_{1 - 2}}{{L}_{1 - 2}} ( {C}_{1} - {C}_{2} ) \\ {\dot{m}}_{2 - 3} = {\dot{m}}_{1 - 2} \\ {\dot{m}}_{3 - ∞} = - k [ {C}_{3} - 0 ] {MV}_{3} + {\dot{m}}_{2 - 3} = \frac{\partial {C}_{3}}{\partial t} {MV}_{3} , \qquad ( 28 )$$

where M is the molecular weight, D_{AB} is the diffusion coefficient of DNA in water, A₁₋₂ is the cross-sectional area of the valve, L₁₋₂ is the length of the valve, V₃ is the volume of the mixing chamber, C₁, C₂, and C₃ are the concentrations at the start and end of the valve and the mixing chamber respectively, and k is the reaction rate. Assumptions about the concentrations are made. C₁ is considered a constant and C₂=C₃. Applying these assumptions and combining the mass flow rates

$$\frac{\partial {C}_{3}}{\partial t} {V}_{3} M = - {kC}_{3} {MV}_{3} + \frac{{MD}_{AB}}{{L}_{1 - 2}} ( {C}_{1} - {C}_{2} ) \\ \frac{\partial {C}_{3}}{\partial t} = - {kC}_{3} + \frac{{D}_{AB} {A}_{1 - 2}}{{V}_{3} {L}_{1 - 2}} ( {C}_{1} - {C}_{3} ) \qquad ( 29 )$$

results in the steady background which needs to be linearized for perturbations

$$\frac{\partial {C}_{3}^{′}}{\partial t} = - {kC}_{3}^{′} + \frac{{D}_{AB}}{{V}_{3} {L}_{1 - 2}} ( {A}_{1 - 2}^{′} {\overline{C}}_{1} - {\overline{A}}_{1 - 2} {C}_{3}^{′} ) . \qquad ( 30 )$$

Rearranging Equation (30) and taking the Laplace Transform gives

$$\frac{{C}_{3}^{′}}{{\overline{C}}_{1}} s + ( k + \frac{{D}_{AB} {\overline{A}}_{1 - 2}}{{V}_{3} {L}_{1 - 2}} ) \frac{{C}_{3}^{′}}{{\overline{C}}_{1}} = \frac{{D}_{AB}}{{V}_{3} {L}_{1 - 2}} \frac{{A}_{1 - 2}^{′}}{{A}_{ref}} {A}_{ref} , \qquad ( 31 )$$

which can be further rearranged to give

$$\frac{{C}_{3}^{′} / {\overline{C}}_{1}}{{A}_{1 - 2}^{′} / {A}_{ref}} = \frac{\frac{{D}_{AB} {A}_{ref}}{{V}_{3} {L}_{1 - 2}}}{s + k + \frac{{D}_{AB} {\overline{A}}_{1 - 2}}{{V}_{3} {L}_{1 - 2}}} . \qquad ( 32 )$$

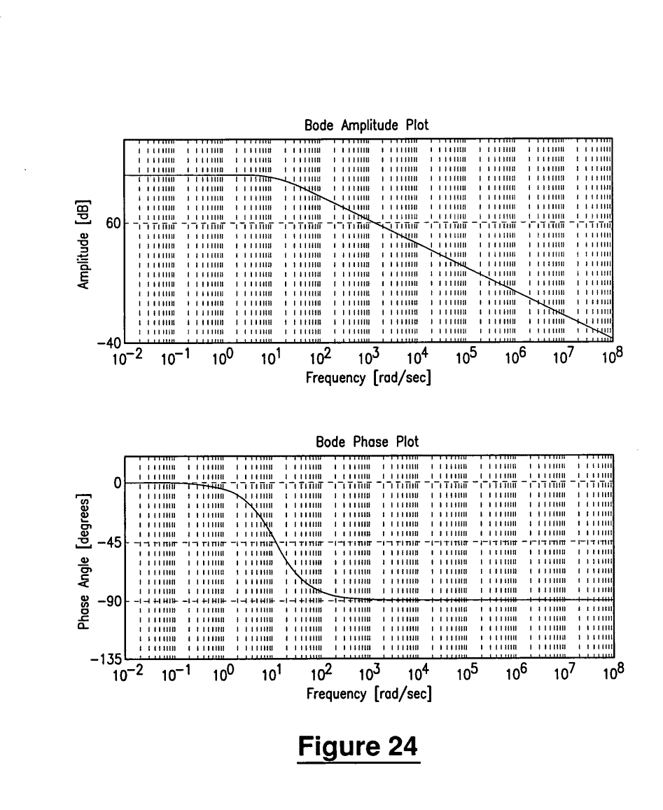

This equation is the ratio of the percent change of concentration to cross-sectional area and the transfer function for the chemical dynamics. Bode plots of the transfer function were generated (FIG. 24).

The gain for the chemical dynamics is

$$K = \frac{{D}_{AB} {A}_{ref}}{k {V}_{3} {L}_{1 - 2} + {D}_{AB} {\overline{A}}_{1 - 2}} , \qquad ( 33 )$$

and the bandwidth is

$$\frac{1}{τ} = \frac{k {V}_{3} {L}_{1 - 2} + {D}_{AB} {\overline{A}}_{1 - 2}}{{V}_{3} {L}_{1 - 2}} . \qquad ( 34 )$$

Combining the mechanical and chemical dynamics results in a transfer function of

$$\frac{C ( s )}{G ( s )} = \frac{1}{{s}^{2} + 2 {ζω}_{n} s + {ω}_{n}^{2}} \cdot \frac{\frac{{D}_{AB} {A}_{ref}}{{V}_{3} {L}_{1 - 2}}}{s + k + \frac{{D}_{AB} {\overline{A}}_{1 - 2}}{{V}_{3} {L}_{1 - 2}}} \qquad ( 35 )$$

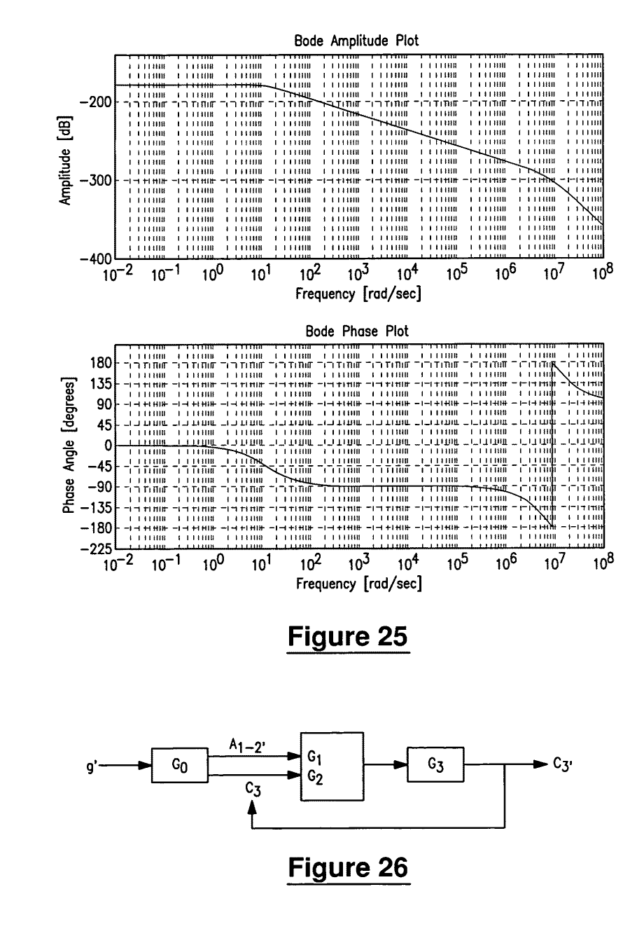

with which the Bode plots in FIG. 25 were obtained.

Based on these results, the model was deemed too idealistic and modifications were made.

IV.B.2. Modified Model

The first approximation of the model neglected the valve dynamics of the device. A more accurate model takes into account these dynamics. The modified block diagram is depicted in FIG. 26. The mechanical dynamics with transfer function, G₀, as an input of the acceleration and output of the valve cross-sectional area. The valve dynamics require two transfer functions, G₁ and G₂, with inputs of the valve cross-sectional area and concentration of the mixing chamber, which is obtained as feedback, and output of the mass flow rate out of the valve into the mixing chamber. The chemical dynamics uses this flow rate as the input and outputs the mixing chamber concentration for transfer function, G₃.

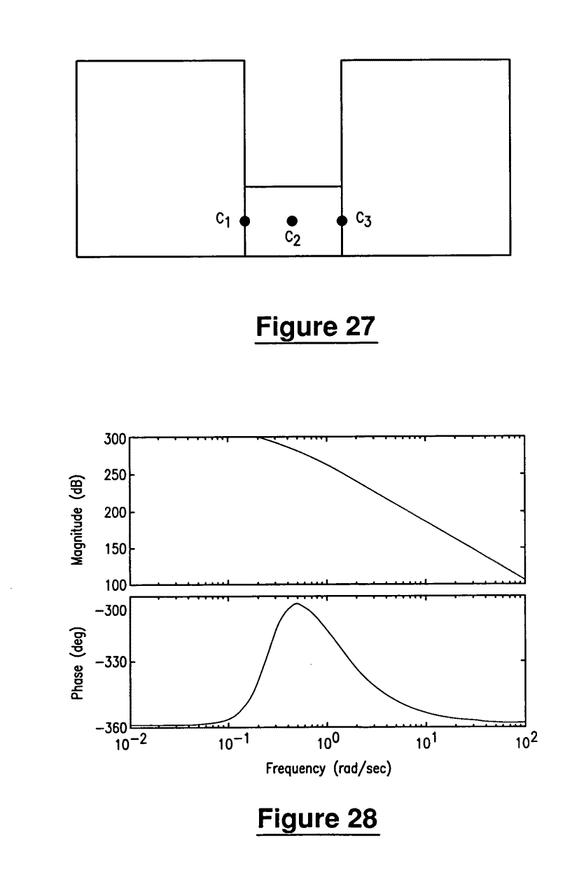

The three concentration points of interest are defined differently from the first approximation and are depicted in FIG. 27. C₁ is defined as the concentration in the holding reservoir and assumed constant; C₂ is the concentration at the midpoint of the valve, and C₃ is the concentration at the entrance to the mixing chamber.

The mass flow into the valve, \dot{m}ᵢₙ, is

$$\begin{matrix}{\dot{m}}_{in} = ( {C}_{1} - {C}_{2} ) \frac{{A}_{1 - 2}}{{L}_{1 - 2} / 2} {D}_{AB} \\ = ( {C}_{1} - ( {\overline{C}}_{2} + {C}_{2}^{′} ) ) \frac{( {\overline{A}}_{1 - 2} + {A}_{1 - 2}^{′} )}{{L}_{1 - 2} / 2} {D}_{AB}\end{matrix} \qquad ( 36 )$$

which has also been linearized for perturbations resulting from the displacement of the cantilever and resulting change in cross-sectional area of the valve, A₁₋₂. The mass flow into the valve can be decomposed into steady, {dot over ( m̅ᵢₙ, and unsteady components, \dot{m}ᵢₙ′,

$${{m}^{\overline{.}}}_{in} = ( {C}_{1} - {\overline{C}}_{2} ) \frac{{\overline{A}}_{1 - 2}}{{L}_{1 - 2} / 2} {D}_{AB} \\ {\dot{m}}_{in}^{′} = ( {C}_{1} - {\overline{C}}_{2} + ) \frac{{A}_{1 - 2}^{′}}{{L}_{1 - 2} / 2} {D}_{AB} - {C}_{2}^{′} \frac{{\overline{A}}_{1 - 2}}{{L}_{1 - 2} / 2} {D}_{AB} \qquad ( 37 )$$

The unsteady mass flow out of the valve, \dot{m}ₒᵤₜ′, is

$${\dot{m}}_{out}^{′} = ( {C}_{2}^{′} - {C}_{3}^{′} ) \frac{{\overline{A}}_{1 - 2}}{{L}_{1 - 2} / 2} {D}_{AB} , \qquad ( 38 )$$

which can be rearranged to yield

$${C}_{2}^{′} = \frac{{\dot{m}}_{out}^{′} + \frac{{C}_{3}^{′} {\overline{A}}_{1 - 2} {D}_{AB}}{{L}_{1 - 2} / 2}}{\frac{{\overline{A}}_{1 - 2} {D}_{AB}}{{L}_{1 - 2} / 2}} = \frac{{L}_{1 - 2} {\dot{m}}_{out}^{′}}{2 {\overline{A}}_{1 - 2} {D}_{AB}} + {C}_{3}^{′} . \qquad ( 39 )$$

Establishing the relationship

$${V}_{1 - 2} \frac{\partial {C}_{2}^{′}}{\partial t} = {\dot{m}}_{in}^{′} - {\dot{m}}_{out}^{′} , \qquad ( 40 )$$

and substituting in the appropriate relationship in Equation (37) results in

$${V}_{1 - 2} \frac{\partial {C}_{2}^{′}}{\partial t} = ( {C}_{1} - {\overline{C}}_{2} ) \frac{{A}_{1 - 2}^{′}}{{L}_{1 - 2} / 2} {D}_{AB} - {C}_{2}^{′} \frac{{\overline{A}}_{1 - 2}}{{L}_{1 - 2} / 2} {D}_{AB} - {\dot{m}}_{out}^{′} , \qquad ( 41 )$$

which can be rearranged to

$${\dot{m}}_{out}^{′} = - {V}_{1 - 2} \frac{\partial {C}_{2}^{′}}{\partial t} - {C}_{2}^{′} \frac{{\overline{A}}_{1 - 2}}{{L}_{1 - 2} / 2} {D}_{AB} + ( {C}_{1} - {\overline{C}}_{2} ) \frac{{A}_{1 - 2}^{′}}{{L}_{1 - 2} / 2} {D}_{AB} . \qquad ( 42 )$$

Substituting Equation (39) into Equation (42) and simplifying results in

$$\frac{{V}_{1 - 2} {L}_{1 - 2}}{2 {\overline{A}}_{1 - 2} {D}_{AB}} \frac{\partial {\dot{m}}_{out}^{′}}{\partial t} + {\dot{m}}_{out}^{′2} = - {V}_{1 - 2} \frac{\partial {C}_{3}^{′}}{\partial t} - \frac{2 {\overline{A}}_{1 - 2} {D}_{AB} {C}_{3}^{′}}{{L}_{1 - 2}} + \frac{2 {A}_{1 - 2}^{′} {D}_{AB}}{{L}_{1 - 2}} ( {C}_{1} - {\overline{C}}_{2} ) . \qquad ( 43 )$$

Taking the Laplace Transform of Equation (43) gives

$$\frac{{V}_{1 - 2} {L}_{1 - 2}}{2 {\overline{A}}_{1 - 2} {D}_{AB}} \frac{\partial {\dot{m}}_{out}^{′}}{\partial t} + {\dot{m}}_{out}^{′2} = - {V}_{1 - 2} \frac{\partial {C}_{3}^{′}}{\partial t} - \frac{2 {\overline{A}}_{1 - 2} {D}_{AB} {C}_{3}^{′}}{{L}_{1 - 2}} + \frac{2 {A}_{1 - 2}^{′} {D}_{AB}}{{L}_{1 - 2}} ( {C}_{1} - {\overline{C}}_{2} ) , \qquad ( 44 )$$

which can be used to find the two transfer functions of the valve dynamics, G₁ and G₂,

$$\frac{{\dot{m}}_{out}^{′}}{{A}_{1 - 2}^{′}} = \frac{4 {D}_{AB}^{2} {\overline{A}}_{1 - 2} ( {C}_{1} - {\overline{C}}_{2} )}{2 {\overline{A}}_{1 - 2} {L}_{1 - 2} {D}_{AB} {s}^{2} + {V}_{1 - 2} {L}_{1 - 2}^{2} s} \\ \frac{{\dot{m}}_{out}^{′}}{{C}_{3}^{′}} = \frac{- 2 {\overline{A}}_{1 - 2} {D}_{AB} ( 2 {\overline{A}}_{1 - 2} {D}_{AB} + {V}_{1 - 2} {L}_{1 - 2} s )}{2 {\overline{A}}_{1 - 2} {L}_{1 - 2} {D}_{AB} {s}^{2} + {V}_{1 - 2} {L}_{1 - 2}^{2} s} . \qquad ( 45 )$$

The chemistry dynamics are defined as

$${V}_{3 } \frac{\partial {C}_{3}^{′}}{\partial t} = {\dot{m}}_{out}^{′} - k {V}_{3} {C}_{3}^{′} , \qquad ( 46 )$$

which says that the mass in the mixing chamber is the mass flow out of the valve and into the chamber minus the mass consumed in the binding reaction. The transfer function, G₃, is

$$\frac{{C}_{3}^{′}}{{\dot{m}}_{out}^{′}} = \frac{1}{{V}_{3} ( s + k )} . \qquad ( 47 )$$

The combined dynamics of the valve and chemistry results in the transfer function

$$\frac{{C}_{3}^{′}}{{A}_{1 - 2}^{′}} = \frac{2 {D}_{AB} ( {C}_{1} - {\overline{C}}_{2} )}{\begin{matrix}{V}_{3} {L}_{1 - 2} {s}^{4} + {V}_{3} {L}_{1 - 2} ( \frac{{V}_{1 - 2} {L}_{1 - 2}}{2 {\overline{A}}_{1 - 2} {D}_{AB}} + k ) {s}^{3} + \\ \frac{{V}_{1 - 2} {L}_{1 - 2}}{2 {\overline{A}}_{1 - 2} {D}_{AB}} {V}_{3} {L}_{1 - 2} {ks}^{2} + {V}_{1 - 2} {L}_{1 - 2} s + 2 {\overline{A}}_{1 - 2} {D}_{AB}\end{matrix}} . \qquad ( 48 )$$

This transfer function produces the Bode plots in FIG. 28.

This model shows a lower cut-off frequency than the first approximation due to the time lag related to the DNA segments traveling through the valve. The gain of the combined chemical and valve dynamics is

$$K = \frac{2 {D}_{AB} ( {C}_{1} - {\overline{C}}_{2} )}{2 {\overline{A}}_{1 - 2} {D}_{AB}} = \frac{( {C}_{1} - {\overline{C}}_{2} )}{{\overline{A}}_{1 - 2}} , \qquad ( 49 )$$

and the bandwidth is calculated numerically using Matlab.

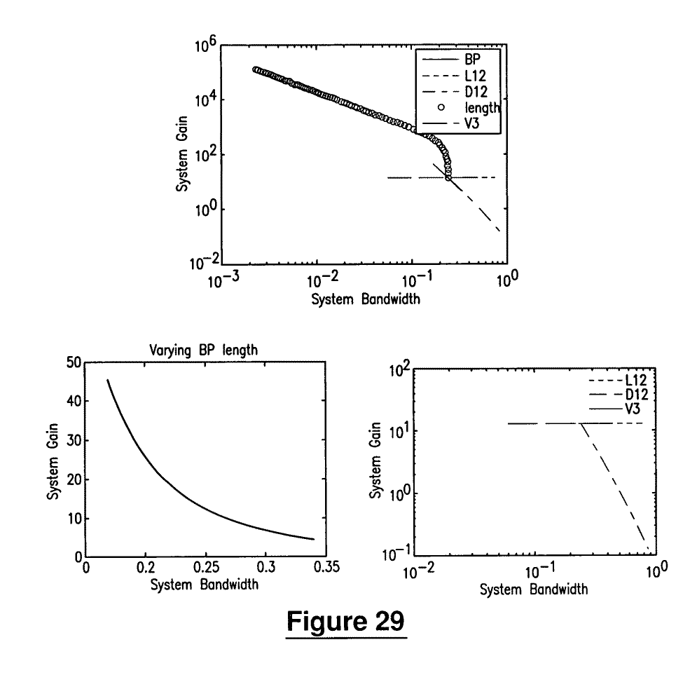

A parametric sensitivity study was completed by varying different parameters and plotting the gain and bandwidth. The parameters studied are the length of the cantilever, the number of base pairs the DNA segment contains and resulting diffusion coefficient, the length and diameter of the valve, and the volume of the mixing chamber. FIG. 29 is the result from varying the different parameters.

Increasing the length of the cantilever or the number of base pairs in the DNA segment, decreases the bandwidth and increases the gain; increasing the length of the valve or the volume of the mixing chamber decreases the bandwidth, but has no impact on the gain; and increasing the diameter of the valve increases the bandwidth and decreases the gain. This result means that the device has a lower gain, but larger bandwidth when the length of the cantilevers, dimensions of the valve, and volume of the mixing chamber are minimized. The opposite is true for the diameter of the valve—minimizing the diameter of the valve results in a smaller bandwidth and higher gain. Ideally, the device would be made as small as possible dimensionally while preserving the largest possible valve diameter within that constraint.

IV.C. Accelerometer Comparison

An important aspect to determining the feasibility of the presently disclosed device is comparing the projected characteristics such as the gain and bandwidth to current commercially available accelerometers.

IV.C.1. System Gain and Bandwidth

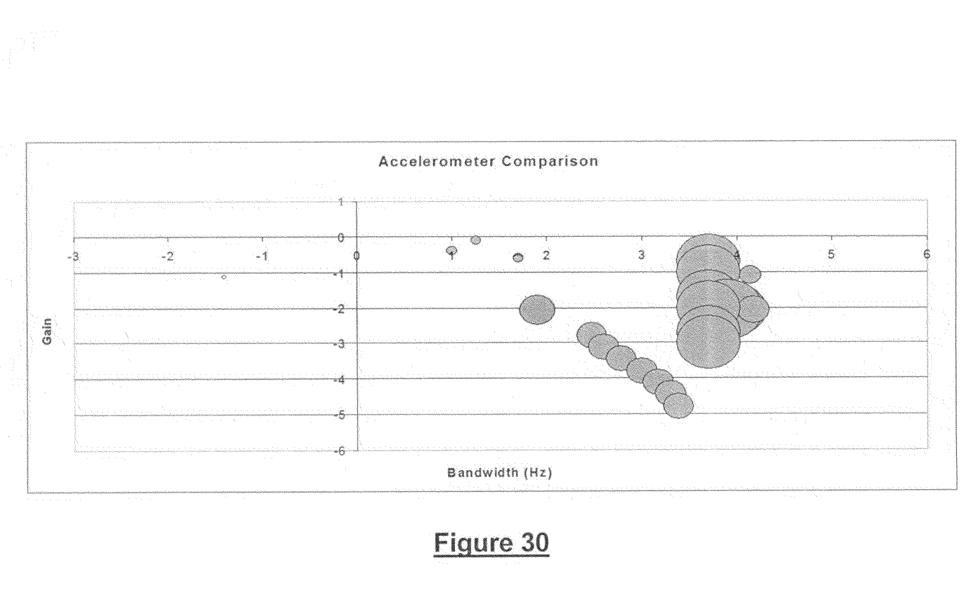

The instantly described device has a bandwidth of 0.247 rad/s and a gain of 12.8 dB. FIG. 30 compares the instantly described device, denoted as a star, to other commercially available accelerometers of various sizes and range capabilities. The graph displays the bandwidth, gain, and relative size of the accelerometers. The instantly described device, however, has such a small size compared to the other accelerometers that its size had to be increased for viewing purposes.

The instantly described device has a smaller bandwidth compared to conventional accelerometers due to the diffusion times of the DNA segments from the holding reservoir to the mixing chamber and the chemical binding of the DNA segments. The gain of the instantly described device is similar to many of the conventional accelerometers. The greatest advantage of the instantly described device is its size. The instantly described device is more than 12 orders of magnitude smaller than any of the current accelerometers.

IV.C.2. Packing Density

As an initial approximation, an 18 bp DNA segment was modeled as a sphere of radius 4 nm using the Stokes-Einstein relationship. Spheres have a packing density of approximately 75%; therefore, in the proposed mixing chamber about 3×10⁶ DNA segments could be recorded. The rougher the resolution of the acceleration recording or number of DNA segments used to record one second of acceleration, the longer the device's history storage capability; the finer the resolution, the shorter the history storage capability.

IV.D. Integration Challenges

There are several potential integration challenges between the use of cantilevers to sense acceleration and chemistry to record the position. Designing and fabricating the device to accommodate the chemistry can be challenging. The chemistry can require a unique reservoir for each cantilever, an opening through which the unit can travel upon deflection, and a main chamber for the units to combine. Ensuring the chemical units are released at a specific time rate upon deflection of the cantilevers can require careful design. Another challenge when the device is fully integrated on the desired size scale is how to review the data that the chemistry has recorded.

### V. Chemical Sensing of Temperature

V.A. General Considerations

Also provided herein are nanodevices for sensing and chemical recording that comprise one or more temperature-sensitive porous liposomes (the reservoirs), the pores of which (the valves) open only above a specified transition temperature. The mixture can contain two or more varieties of liposomes (e.g., 2, 3, 4, 5, 6, 7, 8, 9, 10, or more, depending only on the number of different temperature values are designed to be assayed), with each variety corresponding to a unique transition temperature. Each liposome can be loaded with a quantity of a recording unit selected from, but not limited to, short DNA segments with overhanging “sticky ends” or nucleotides. The sequence of the DNA segments or specific nucleotide for each variety of liposome can be unique to that variety of liposome and identical for all liposomes of that variety. The solution that surrounds the liposomes can also contain appropriate enzymes (e.g., DNA and/or RNA ligase) as well as any other reagents that might be required to allow for the ligation and/or polymerization of the DNA segments or nucleotides, providing a mechanism for the assembly of available segments into a long chain. The mixture can be contained in any of a variety of containers (the reaction chambers), including, but not limited to, a test tube, a microcavity, a large liposome or vesosome, or a cell.

Terminal deoxynucleotidyl transferase (TdTase) is another option for binding together the unique payloads of thermally-sensitive liposomes. TdTase is a template independent polymerase that adds mononucleotides to the 3′ end of DNA segments. Instead of using longer sequences of DNA which could potentially be too large for the liposome pores, TdTase allows for the use of nucleotides which could be released easily.

Given this design, the time history of the temperature experienced by the device can be recorded in some embodiments, as follows: (1) when the temperature is at its initial level, only those liposomes for which the threshold temperature falls below the initial temperature have open pores and the recording units (e.g., the DNA) stored in these liposomes diffuses into the immediate surroundings (e.g., into a reaction chamber) while the DNA present in liposomes with higher threshold temperatures remains locked within the liposomes and thus is unable to diffuse to and/or enter the reaction chamber; (2) the enzyme (e.g., the ligase) assembles the available DNA segments into a long DNA chain as they become available, thereby recording information on the temperature experienced; (3) if the temperature is raised, new liposomes open and the associated DNA sequences also become incorporated into the chain as it assembly continues, whereas if the temperature is lowered, the high-threshold liposome pores close and the high-temperature segments are no longer incorporated; (4) by sequencing the DNA strands that are generated by the ligase activity over the course of a “run”, it becomes possible to deduce the time history of the temperatures experienced by the device from the statistics of the strand sequence. It is noted that since there are multiple strands that are likely to be formed, the sequencing is also expected to be statistical.

In some embodiments, the instant approach takes advantage of the use of vesosomes, which are large lipid bilayer structures that encapsulate other liposomes. The liposomes that harbor the recording units (e.g., the DNA) as described hereinabove can be further encapsulated in a vesosome, wherein the vesosome has a higher transition temperature than any liposome it encapsulates to ensure that its contents are not released during operation. Essentially, in some embodiments, the vesosome acts a container that replaces the test tube in the lab. The ligase can thus be either in the encapsulated solution of the vesosome or can lie in a separate small liposome with the lowest transition temperature, ensuring that it is released first during operation. The vesosome can subsequently enter cells where it can pass on its genetic memory, thus ensuring that while the “machinery” might die (i.e., cease to be functional as a unit), the “memory” continues as a record inside an infected cell.

Furthermore, the liposomes of the presently disclosed subject matter are not limited to only temperature sensitivity as the method of release. In some embodiments, the liposomes are sensitive to various environmental conditions including, but not limited to, light, pH of the microenvironment, ultrasound, and enzymes.

V.B. Temperature-Sensitive Liposomes

Bioinspired nanotechnology has drawn many materials and lessons from nature. The protective coating of every living cell is a lipid bilayer which has many functions including transport across the membrane, recognition, and signaling. Lipids self-assemble in an aqueous environment into vesicles termed liposomes (Szoka Jr. et al., 1980). For the past 40 years, liposomes have been used as drug delivery devices, encapsulating a solution of interest with the end goal of delivering a therapeutic payload in vivo (Gaber et al., 1996; Needham et al., 2000; Needham & Dewhirst, 2001; Ponce et al., 2007). Currently marketed liposomes lack active targeting and rely on passive diffusion and nonspecific degradation of the liposome (Andresen et al., 2004). As a result, active targeting and triggered release have been pursued to maximize payload delivery to the area of interest.

Any hydrophobic molecule stored in the bilayer itself or hydrophilic molecule stored in the aqueous core can be carried within liposomes including chemotherapeutic agents, MRI contrast agents, or for example and as disclosed herein, nucleic acids. Many obstacles can impede the liposomal delivery system and their properties can be modified to characterize liposomes as a system for triggered release. These issues include, for example, loading high concentrations of reagents into the aqueous core of the liposome, as many reagents and ions can present barriers to this task. Stability of liposomes, in vivo and in vitro, can be qualified, and if inadequate, the composition or protocol for loading can be altered. Scientific reproducibility and mass producing in industry can benefit from a standardized product, and thus the size of the liposomes is in some embodiments, uniform between batches.

Finally, release of the encapsulated reagent(s) must be achieved under appropriate conditions. Much of the current research in the field relates to delivery of chemotherapeutics in a tumor microenvironment and as a result, the specificity of release is well-characterized. Active targeting includes labeling the outside of the liposome with any receptor over-expressed in the delivery tissue of interest, such as folate receptors, integrin surface receptors, or with the use of antibodies (Allen et al., 1995; Arap et al., 1998; Park et al., 2001; Hood et al., 2002; Lu & Low, 2002; Park et al., 2002; Lu & Low, 2003; Sapra & Allen, 2003; Aronov et al., 2003). Release can also be triggered by an external stimulus, such as light (Shum et al., 2001), pH of the microenvironment (Yatvin et al., 1980a, 1980b; Drummond et al., 2000), ultrasound (Unger et al., 1998), enzymes (Vermehren et al., 1998; Andresen et al., 2004), or temperature (Iga et al., 1993; Needham et al., 2000; Needham & Dewhirst, 2001).

Temperature-sensitive liposomes (TSL) thus have the ability to release their contents quickly at specific temperatures based on the composition of the liposomes.

V.B.1. Composition

Most TSL are made of several different lipids to give the liposome different properties. However, nearly every TSL uses a neutrally charged 16-carbon phospholipid, 1,2-dipalmitoyl-sn-glycero-3-phosphocholine (DPPC) as the major component of the membrane (Shum et al., 2001). A phospholipid has two fatty acid (nonpolar) tails and a polar head (the phosphate and head group, in this case, choline). DPPC has a transition temperature of anywhere from 41.0 to 41.5° C. (Kong & Dewhirst, 1999; Ono et al., 2002; Chiu et al., 2005). To change the transition temperature, other lipids are included. The simplest changes come from including other phosphatidylcholines of different carbon chain length, such as but not limited to 1,2-dimyristoyl-sn-glycero-3-phosphocholine (DMPC), a 14-carbon phospholipid with a transition temperature of 34° C., and 1,2-dimyristoyl-sn-glycero-3-phosphocholine (DSPC), an 18-carbon phospholipid with a transition temperature of 54° C. By mixing lipids in different molar ratios, the transition temperature can be shifted between the main transition temperatures of the individual lipids alone.

For in vivo applications, polymers conjugated to lipids can be added to the composition of the liposome to prolong circulation time. By way of non-limiting example, 1,2-distearoyl-sn-glycero-3-phosphoethanolamine-N-polyethathleneglycol 2000 (DSPE-PEG 2000) can be employed (Unezaki et al., 1994). DSPE-PEG 2000 has a 2000 dalton (Da) molecular weight chain of poly(ethylene glycol) which has a subunit of -[-o-CH₂—CH₂—]—. Each subunit has a weight of 44.05 Da. Modeled after the red blood cell that has a 1400 angstrom layer of glycolax, the pegalated lipid serves as a steric boundary for the liposome to prevent it from being taken up by the reticuloendothelial system (RES) of the liver and kidneys that protects the body from foreign particles and pathogens with an opsonization system.

It has been shown that the inclusion of lysolipid in the composition can dramatically increase the peak of the transition temperature and release rate (Mills & Needham, 2005). Lysolipids are different from phospholipids in that they have only one fatty acid chain and therefore can create micelles with a higher curvature and lower concentration required. The critical micelle content for the lysolipid used is typically about 3 μM, above which the lysolipid generally separates from the bilayer and creates micelles. Two different exemplary lysolipids that can be employed are 1-palmitoyl-2-hydroxy-sn-Glycero-3-phosphocholine (MPPC), a 16-carbon unsaturated fatty acid, and 1-stearoyl-2-hydroxy-sn-glycero-3-phosphocholine (MSPC), an 18-carbon unsaturated fatty acid. Both of these lysolipids lower the transition temperature when included with DPPC (Ponce, 2007).

Cholesterol is generally not included in the formulation of thermosensitive liposomes because at cholesterol concentrations greater than 25-30 mole percent, the enthalpy required for the transition to take place is reduced or even eliminated such that no transition or increase in permeability is observed (Magin & Niesman, 1984; Sandstrom et al., 2005). Efforts have been made to optimize the amount of cholesterol included in most conventional liposomes because of the benefit of additional membrane stability that was expected (Gregoriadis & Davis, 1979; Kirby et al., 1980; Weinstein et al., 1980; Bassett et al., 1986; Merlin, 1991; Unezaki et al., 1994; Gaber et al., 1995). However, PEG lipid is seen to give the liposome steric stability, in addition to avoiding the RES (Unezaki et al., 1994; Dos Santos et al., 2002).

V.B.2. Characterization

The phase transition of thermosensitive liposomes can be measured with differential scanning calorimetry (DSC; Mills & Needham, 2004). In differential scanning calorimetry, the sample and a standard are slowly heated while the temperature and the energy needed to increase the temperature are measured. When the energy to heat the sample and the energy to heat the standard differ, the sample is undergoing either an exothermic or endothermic reaction depending on the energy input needed to maintain equal temperatures. Using the DSC method, the phase transitions can be determined, as during slow heating from 30-50° C. (for DPPC for example, with a phase transition near 41° C.), much more energy is needed to change the temperature of the DPPC sample than the standard, which is usually hydration buffer or water. The “peak” of the energy input defines the transition temperature, while the start to end of the peak define the range of the transition. Ono et al., 2002 discloses using 900 light scattering measurement at 400 nm to find the pre-transition and main-transition temperature. The pre-transition temperature measured was about 1° C. different from those measured with DSC, while the main transition was closer for pure DPPC and DSPC.

Papahadiopoulos et al., 1973 disclosed that phospholipid bilayers heated through their phase transitions had increased permeability to small molecules at that temperature. Mills & Needham, 2004 discloses an assay to measure the permeability of liposomes to ions as a function of temperature. Briefly, 1.0 mol % NBD-PE was exchanged with 1.0 mol % of the primary lipid (DPPC) in the liposome composition. The nitro group on the NBD-PE absorbs ultraviolet light especially at a 465 nm wavelength. This absorbance is quenched by the addition of the dithionite ion (S₂O₄²⁻) or radical (.SO₂⁻) which exist in equilibrium. The nitro group on the polar head of the lipid is converted to an amine when it comes in contact with the dithionite ion. The NBD labeled formulation, as well as the unlabeled formulation, are prepared by thin film hydration and extrusion and diluted to 3 μM in 509 mM sucrose/NaCl buffer to equilibrate osmolarity. The two samples are equilibrated at temperature in a UV spectrometer and dithionite is added. The permeability is recorded as the outside of the liposomes is quenched first from the NBD on the outer membrane, and the permeability through the membrane is directly proportional to the absorbance quenching. From an exponential model fit of the internal quenching, the permeability rate constant can be found.

V.B.3. Thin Film Method and Extrusion

Liposomes can be formed by the reverse-phase evaporation method (Szoka & Papahadjopoulos, 1978; Kong & Dewhirst, 1999; Ono et al., 2002) or thin lipid extrusion method (Olson et al., 1979; Mayer et al., 1986; Lindner et al., 2004). The thin film method is detailed as follows. Lipids are dissolved and stocked in chloroform in free lipid form. The desired mixture of lipids is added directly to a clean round-bottom flask. The mixture is dried to a thin film using a Rotoevaporator. The flask is covered with aluminum foil, vacuum was pulled for 30 minutes and then the desiccator is sealed off, turning off the vacuum and letting the thin film sit 6 hours or overnight.

Liposome extrusion can be employed to generate unilamilar, same-sized particles. An exemplary procedure is as follows. The lipid film is hydrated with an aqueous solution that forms multilamellar liposomes between 100-800 nm (Mills & Needham, 2004). The water bath to be used for the extruder and hydration is set to about 9° C. above the transition temperature. The round bottom flask is taken out of the desiccator and the DNA buffer solution is added to achieve the desired concentration of lipid. A clean Teflon stir bar is gently swirled in the flask at the appropriate temperature bath for 10-15 minutes to hydrate the lipids. Sonication can also be used alone to make similar sized liposomes (Hayashi et al., 1998), or become a step before extrusion to reduce particle size slightly and extrusion easier.

Next, the extruder is assembled with a drain disk and two polycarbonate

100 nm filters, which can be employed to achieve liposomes of about 100 nm in diameter. A recirculating water bath is attached to the thermobarrel of the extruder to keep the temperature of the liposomes above the transition temperature during extrusion. If the temperature falls to the transition temperature or below, the liposomes can clog the extruder. The buffer solution used to hydrate the thin film is used to wet the filters; then the hydrated lipid solution is extruded 5-10 times through the thermobarrel using nitrogen pressure regulated at 150-200 psi. After the last extrusion, the liposomes can be stored at 4° C. for at least 10-15 minutes to ensure the liposomes anneal into the gel phase.

V.B.4. Loading

Loading into the liposomes can be achieved in numerous ways, either actively, passively, or by utilizing equilibrium on the inside and outside of the liposome. Active loading uses a pH gradient between the inside and the outside of the liposome to draw amphipathic drugs into the aqueous core of the liposome. This method has been used to maximize loading the chemotherapy drug, Doxorubicin (Dox), into the liposome with a citrate buffer. By using a buffer with low pH, like citrate buffer at pH 4, a pH gradient is created as the external solution is taken away and replaced with a buffer near pH 7 (Unezaki et al., 1994; Chiu et al., 2005). When the Dox is added to the solution, it is pulled into the liposome and protonated, unable to escape. Passive loading is achieved by hydrating the dried lipid thin film with the solution to be encapsulated. After extrusion, the external solution is removed using gel filtration columns, and the desired solution is sequestered in the liposomes. Equilibrium loading uses preformed liposomes heated to their transition temperature in a bathing solution that is to be encapsulated. The increased permeability at this temperature allows the interior and exterior solutions to come to equilibrium. The liposomes are cooled below their transition temperature, the elevated permeability no longer exists, and the external solution can be removed by gel filtration leaving the intact liposomes with the solution of interest encapsulated.

V.B.5. Mechanism of Release

The release of thermosensitive liposomes can occur at the transition temperature due to leaky interfacial membranes (Papahadjopoulos et al., 1973; Needham & Dewhirst, 2001). In lysolipid-containing thermosensitive liposomes (LTSL), individual gel phase plates are separated by grain boundaries that occur in the L_{β′} phase of phospholipid membranes when the temperature is decreased from above to below the transition temperature (Ickenstein et al., 2003). The lysolipid enhances the permeable defect structures, either by desorbing rapidly from boundary regions or by stabilizing porous defects. In LTSL, phase transition has been recorded to increase by 20-fold compared to DPPC liposomes (Needham et al., 2000). Ickenstein et al., 2003 proposed that lysolipid and lipid-conjugated PEG accumulates in grain boundaries and that these two micelle forming components (lysolipid and PEG) cause disk formation, as well as, open liposomes, and pore-like defects in LTSL during cycling through the phase transition. However, no disks were present with liposomes containing lysolipid, but lacking lipid-conjugated PEG. Sandström et al., 2005 discloses an assay that has shown that 50% of the lysolipids desorb from LTSL into multilamellar vesicles when incubated at 37° C. U.S. Pat. Nos. 6,726,925; and 6,964,778 also disclose compositions comprising temperature-sensitive liposomes and methods of making and using the same, and are incorporated by reference herein in their entireties.

### VI. Compositions Comprising Pluralities of Reservoirs and Reaction Chambers

In some embodiments, the presently disclosed subject matter provides compositions comprising a plurality of reservoirs and one or more reaction chambers. In some embodiments, the reservoirs are liposomes (e.g., temperature-sensitive liposomes as disclosed herein) that encapsulate one or more chemical units. The reservoirs are designed to release one or more of the chemical units contained therein if and only if the reservoirs experience an environmental state variable above a threshold value. Once released, the chemical units migrate to the reaction chamber in which they are polymerized to generate a polymer of chemical units, the order and number of chemical units present in the polymer being reflective of the environmental state variable experienced by the plurality of reservoirs, including any changes in the environmental state variable experienced by the plurality of reservoirs.

In some embodiments, the plurality of reservoirs comprises temperature-sensitive liposomes (also referred to herein as “thermosensitive” liposomes). In some embodiments, the plurality of reservoirs comprises a plurality of classes of temperature-sensitive liposomes, with each member of a given class of temperature-sensitive liposomes being characterized by a different minimum temperature at which the chemical units encapsulated therein are released.

The plurality of reservoirs and the reaction chamber(s) can be encapsulated with a single closed structure. In some embodiments, the plurality of reservoirs are themselves encapsulated within a lipid bilayer structure called a vesosome. Vesosomes are described in U.S. Pat. Nos. 6,221,401 and 6,565,889 and also in Lasic, 1997 and Walker et al., 1997. Each of these references is incorporated by reference herein in its entirety.

### VII. Other Applications

In some embodiments, an engineered microorganism that can be used as a sensor for tagging, tracking, and locating targets is provided. The engineered microorganism accomplishes these goals by being able to: (1) sense and store the time history (the time series) of an environmental state variable in genetic material for subsequent access and recall by sequencing and/or transcription; (2) transmit this stored information to its offspring; and (3) act on this stored information, including being able to edit the genetic material of its offspring on the basis of the stored information. Potential applications include advanced nanomedicines that incorporate active-feedback mechanisms, microorganisms with adaptively-controlled genomes for drug discovery and/or manufacture, and advanced low-power sensors for traditional engineering applications.

In some embodiments, a high-volume DNA copy machine is provided. The DNA copy machine accomplishes its function by using the information encoded in the nucleotide sequence of the original DNA strand to specify the level of one or more environmental variables such as pH, chemical concentration, or other such variables. These in turn regulate the release of nucleotides from the reservoirs by the mechanisms already discussed to yield multiple copies of a new DNA strand whose contents are a replica of the original strand.

In some embodiments, a strategy for de novo synthesis of long DNA strands is provided. In this embodiment, shorter segments of de novo DNA are synthesized by using fluctuations in an environmental variable such as light or temperature to drive the recording of a specific sequence of DNA nucleotides as in the embodiments already described. These short segments are then replicated in large volume using standard PCR or some other such amplification method before being chained into longer segments to yield a long de novo DNA strand using PCA and PCR or some other such standard method. These embodiments would provide a mechanism for directly writing a de novo genome for a cell or microorganism.

In some embodiments, a strategy for de novo synthesis of polypeptides and proteins is provided. In some embodiments, this is accomplished by augmenting A method for sensing and chemical recording into genetic material with a method for translation of the information stored in the genetic material into a protein or protein complex. In some embodiments, this is accomplished through the use of ribosomes and associated cofactors and reagents, which can be stored in the same reaction chamber as the ligase, or can be stored in a separate reaction chamber, access to which is governed by an environmental state variable via a mechanism like that already described for the governing of the release of chemical subunits from storage reservoirs or by some other mechanism. In some embodiments, ribosomes are used to operate on the DNA written by the chemical recording process, combining amino acids available in the ambient surroundings into polypeptides. In some embodiments, ribosomes are in the reaction chamber. In some embodiments, a collection of ribosomes, amino acids, and cofactors are stored in a separate reservoir, access to which is regulated by valves mechanisms.

In some embodiments, a strategy for audio sensing and recording is provided in the form of a nano-scale acoustic sensor with memory. In this embodiment, the environmental variable which governs the chemical recording process is sound level and the record provided in the long chain polymer written by this chemical process is a time-series record of the sound levels detected.

In some embodiments, a strategy for optical sensing and recording is provided in the form of a nano-scale optical sensor with memory. In this embodiment, the environmental variable which governs the chemical recording process is the intensity of incident light or electromagnetic energy and the record provided in the long chain polymer written by this chemical process is a time-series record of the light levels detected. This embodiment could constitute, for example, a focal plane array.

In some embodiments, a strategy for holographic archival data storage is provided by arraying multiple layers of the sort described by the preceding paragraph into a multi-layer structure.

In some embodiments, a strategy for de novo synthesis of a genome for subsequent insertion into a virus, natural organism, or artificial organism is provided. This is accomplished by implementing one of the already described strategy for sensing and recording the levels of one or more environmental state variables into a DNA sequence or other type of genetic material, and then manipulating the sensed environmental variable so as to write a DNA strand containing nucleotide sequences that correspond to meaningful genes.

In some embodiments, a “multi-spectral” sensor is provided. This multispectral sensor would incorporate into a single reaction chamber a number of types of liposome, each type of which is sensitive to a different environmental variable, enabling simultaneous sensing of temperature, light levels, pH levels, sound levels, protein presence, etc.; and the simultaneous recording of the levels of all of these sensed variables into the same DNA chain or other polymer chain.

In some embodiments, a strategy for producing an organism that exhibits genetic memory is provided. In some embodiments, this is accomplished by implementing a mechanism for sensing and chemical recording where the chemical units are nucleotides, the reservoirs are liposomes, the valves are the pores of these liposomes, the reaction chamber is a liposome or vesosome containing TdTase or ligase, and this complete mixture is contained in a vesosome which is itself contained within the cell. In addition the mixture also contains proteins and cofactors that enable the delivery of the genetic material written in the reaction chamber or chambers into the genome of the host organism via a viral mechanism or some other appropriate mechanism. The device can be inserted into the host cell by micropipette or by lipofection or, in the case of a de novo synthetic organism, as part of the manufacturing process for the de novo organism.

In some embodiments, a strategy for producing an organism with a programmable genome is provided. In some embodiments, this is accomplished by implementing a mechanism for sensing and chemical recording where the chemical units are nucleotides, the reservoirs are liposomes, the valves are the pores of these liposomes, the reaction chamber is a liposome or vesosome containing TdTase or ligase, and this complete mixture is contained in a vesosome which is itself contained within the cell. In addition the mixture also contains proteins and cofactors that enable the delivery of the genetic material written in the reaction chamber or chambers into the genome of the host organism via a viral mechanism or some other appropriate mechanism. The device can be inserted into the host cell by micropipette or by lipofection or, in the case of a de novo synthetic organism, as part of the manufacturing process for the de novo organism. The genes of the host organism can then be programmed by manipulating the environmental variable to which the chemical recording mechanism is sensitive in order to write a desired nucleotide sequence.

In some embodiments, a composition for implementing a microorganism possessed of a genome which is at least part, or in full, programmable is provided. In some embodiments, the microorganism contains within it (a) one or more sensing and chemical recording mechanisms which record the time history of changes to one or more environmental state variables to which the microorganism is exposed into a strand of genetic material in such a way that the nucleotide sequence in the genetic material provides a record of the time series of those environmental variables; (b) a chamber containing a mixture of enzymes and other reagents which are able to incorporate the strand of genetic material written by the chemical recording mechanism into the genetic material of the host microorganism by viral or other mechanisms; and (c) optionally, a mixture of proteins and other factors and cofactors which are able to in some way express the nucleotide sequences of the programmed organism via any one of the usual naturally occurring or laboratory-developed mechanisms by which organisms express genes. In some embodiments, the chemical sensing and recording mechanisms are contained within one or a plurality of vesosomes. In some embodiments, the vesosome is originally inserted into the microorganism using a pipette. In some embodiments, the vesosome is originally inserted into the microorganism using lipofection. In some embodiments, the vesosome is contained in an artificial organism and is originally placed there as part of the process of assembling that microorganism.

In some embodiments, a method for programming all or part of the genome an organism that is capable of recording into genetic material the time history of changes in one or more of the environmental state variables of the environment to which the organism is exposed is provided. In some embodiments, the programming comprises manipulating the relevant environmental state variables in order to yield genetic material with a desired nucleotide sequence.

In some embodiments, a method for volume production of several organisms all of the same species but each possessed of a genome which is in part, or in full, programmable wherein these portions of the genome are independently programmed by exposing each individual organism to unique changes in its environment is provided. In some embodiments, a method is provided for implementing an engineered multicellular organism the genomes of the offspring of which may be programmed by exposing the parent organism to changes in environmental state variables prior to reproduction.

In some embodiments, a method for reading information recorded using a method for recording environmental state variables as described herein is provided. In some embodiments, the reading is accomplished by observing the removal of chemical units from one or both of the ends of one or more of the polymers formed by the recording method. In some embodiments, a reporting signal can comprise a level of fluorescence. In some embodiments, the reading is accomplished by observing the removal of chemical units from one or both of the ends of one or more of the polymers formed by the recording method. In some embodiments, a particular fluorophore is associated with DNA binding so that the detectable fluorescence is altered as units are unbound and/or to particular sequences so that the detectable fluorescence is altered as more chemical units are removed.

In some embodiments, a strategy for producing a multicellular organism the genomes of the offspring of which can be programmed by exposing the parent organism to changes in environmental state variables prior to reproduction is provided. In some embodiments, this is accomplished by implementing a mechanism for sensing and chemical recording that is inside one or more of the germ cells, defines in the broad sense of the term, of the parent organism.

In some embodiments, a strategy for producing a multicellular organism the genomes of the offspring that retain a genetic imprint documenting the time histories of the changes in one or more of the environmental state variables associated with the environments top which the parent organism was exposed over the course of its life up to the point of generation of the relevant offspring is provided. In some embodiments, this is accomplished by implementing a mechanism for sensing and chemical recording that is inside one or more of the germ cells, defines in the broad sense of the term, of the parent organism.

In some embodiments, a strategy for recording the shipping and handling history of a foodstuff, consumer product, seedstock, or some other object is provided. In some embodiments, this is accomplished by coating the product with a biofilm, polymer film, or some other coating containing a mechanism for the sensing and chemical recording of the time history of one or more of the environmental state variables, to include geographical location, of the environments to which the foodstuff or consumer product was exposed. In some embodiments, the coating can be sprayed onto the objects. In some embodiments, the coating can be subsequently sampled to determine the history of the storage, transport, and handling of the object.

In some embodiments, a strategy for recording information on the past locations and experiences of an individual is provided. In some embodiments, this is accomplished by applying to the individual or to the clothing of the individual a particle or particles, or a biofilm, polymer film, or some other object or coating containing a mechanism for the sensing and chemical recording of the time history of one or more of the environmental state variables, to include geographical location, of the environments to which the individual is exposed. In some embodiments, the coating can be applied to the individual surreptitiously. In some embodiments, the particles or coating can be subsequently sampled to determine the history of the storage, transport, and handling of the object.

In some embodiments, a strategy for recording information on the past locations and experiences of a unit of livestock is provided. In some embodiments, this is accomplished by the same mechanisms outlined in the preceding paragraph.

In some embodiments, a strategy for rapidly and inexpensively generating multiple unique identification tags for application to foodstuffs, individuals, or other objects or items of interest is provided. In some embodiments, this is implemented by the writing of a polymer of segment of genetic material in a container, particle, or organism containing a mechanism the sensing and recording of the time history of one or more environmental state variables; and then manipulating these environmental state variables in such a way as to write a desired unique sequence of chemical units.

In some embodiments, a strategy for biomedical diagnostics is provided. In some embodiments, this is accomplished by the detection and recording of protein/receptor binding events. In some embodiments, this is achieved by the use of valves sensitive to such binding events. In some embodiments, this is achieved by the use of protein-sensitive liposomes. In some embodiments, this is achieved by the use of liposomes whose porosity is sensitive to binding events on or in the vicinity of the lipid bilayer of the liposome.

In some embodiments, a sensor for the early detection of very low concentrations of chemical and biological warfare agents is provided. In some embodiments, this is achieved by sensing, recording, and possibly reporting binding events associated with one or a variety of chemical and biological agents.

In some embodiments, a tool for manufacture of synthetic DNA is provided. In some embodiments, this is accomplished photosensitive liposomes containing individual nucleotides which are, on release, assembled into DNA using TdTase. In some embodiments, release of nucleotides is controlled by illuminating the target mixture with different frequencies of light to regulate the release of different nucleotides.

In some embodiments, a tool for data exfiltration that can be used by covert operatives transiting highly-denied areas is provided. In some embodiments, this is accomplished by using liposomes with temperature-sensitive or light-sensitive pores to regulate the chemical recording of a signal originating from a cigarette lighter or flashlight.

In some embodiments, a tool for the process control of pharmaceutical manufacturing is provided. In some embodiments, this is achieved by the inclusion of particles into or films into the constituents of the drugs as they are manufactured in order to provide a complete time history of the experiences of these constituents throughout the manufacturing process.

In some embodiments, a tool for the detection and recording of radiation exposure is provided. In some embodiments, this is achieved through the use of radiation-sensitive liposomes to govern a chemical recording process.

In some embodiments, a strategy for high-density archival data storage is provided. This is accomplished by arraying onto a surface a single layer of reaction chambers into a Cartesian, polar, or other such grid. The reaction chambers would contain the mechanisms for chemical sensing and recording of environmental state information already described in this document. The reaction chambers might be vesosomes bound to the surface or into a gel distributed across the surface, voids or depressions in the material that constitutes the surface, or any other suitable structure. The relevant environmental variable would be temperature, light level, magnetic field orientation, or some other such convenient environmental state variable. This environmental state variable would be manipulated over time in order to write a time sequence of data at each point on the grid. The source of change of the environmental variable might be a mobile write head which is maneuvered over the grid. Or, the grid might be maneuvered under a static write head. Or both the grid and the write head might be maneuvered as in a compact disk writer or DVD writer. The result could be, for example, a disc like a compact disk or DVD where each point illuminated by the writing laser can store one unit of information for each revolution of the disc during the write phase; resulting in a many-orders-of-magnitude increase in the storage density of the disc. The sequence of the starting length of DNA or other polymer at each grid point would be unique to that grid point. The individually written segments could be assembled into a much longer polymer in an order prescribed by the address encoded in their starting lengths.

In some embodiments, methods and compositions for de novo synthesis of DNA are provided. In some embodiments, a very-low-cost mechanism for the de novo synthesis is provided. In some embodiments, this is accomplished with reservoirs that are part of an optical disk that can be exposed to a variety of changes to one or more environmental state variables across different regions of the disk by, for example, writing to the disk using an optical disc writer, and capturing these environmental fluctuations in the form of large quantities of written DNA.

In some embodiments, the chemical recording mechanisms are contained within one or more reaction chambers which each occupy a physical volume. In some embodiments, the reaction chambers can be vesosomes. In some embodiments, the reaction chambers can be polymer shells. In some embodiments, the reaction chambers can consist of depressions pressed, etched, or molded into a disk or wafer made of a sturdy material. In some embodiments, this disk or wafer material can be, but is not limited to be, polycarbonate plastic, glass, or silicon. In some embodiments, this material is polycarbonate plastic. In some embodiments, the reaction chambers are “pits” in a rewritable compact disc, DVD+/-RW disc, BD-RE (Blu-Ray) disc, or other optical disk.

In some embodiments, the floors of each reaction chamber are coated with a material capable of absorbing light. In some embodiments, this light-absorbing material can be, but is not limited to be, an organic dye or a phase change material. In some embodiments, the light-absorbing material is Germanium-Antimony-Tellurium (GST). In some embodiments, the light-absorbing material is Silver-Indium-Antimony-Tellurium. In some embodiments, the floors themselves, or, if one is present, the light-absorbing layer is coated with a layer of reflective material. In some embodiments, this reflective material can be, but is not limited to be, a thin layer of gold, silver, aluminum, copper, or some other metal. In some embodiments, this reflective material is gold. In some embodiments, the reaction chamber contains a mixture of reservoirs containing chemical units; chemical catalysts or other mechanisms capable of chaining these chemical units into a polymer; and, optionally, short lengths of polymer that serves as primers onto which chemical units can be added.

In some embodiments, the reservoirs can be, but are not limited to be, one or more varieties of liposome. In some embodiments, these liposomes can be, but are not limited to be, thermosensitive liposomes, photosensitive liposomes, ph-sensitive liposomes, or liposomes whose porosity is regulated by binding events on the surface of their bilayers. In some embodiments, these reservoirs are four classes of thermosensitive liposomes, each with a different release temperature. In some embodiments, these reservoirs can be, but are not limited to be, proteins capable of undergoing a change in conformation in response to a change in the level of an environmental state variable.

In some embodiments, the reservoirs can be, but are not limited to be, freely floating in solution, or tethered to the walls of the reaction chamber, or tethered to each other, or tethered to multiple locations. In some embodiments, the reservoirs are tethered to the walls of the reaction chamber. In some embodiments, these tethers, if they exist, can be, but are not required to be, made of organic polymers, inorganic polymers, metal wires, or ceramic posts. In some embodiments, the reservoirs are tethered to the wall by streptavidin. In another embodiment, the reservoirs are tethered to the wall by folate. In some embodiments, the reservoirs are tethered to each other by streptavidin.

In some embodiments, the reaction chambers are, but are not required to be, arrayed into geometric patterns, such as a variety of planar Cartesian grids, a variety of planar polar grids, a planar hexagonal close-packed array, or three-dimensional hexagonal close lattice, for convenient access to a source of change of environmental variable or for convenient containment into a physical volume. In some embodiments, the reaction chambers are arrayed as a spiral from a central axis. In some embodiments, the reaction chambers are arrayed along the standard spiral track of a DVD disk. In some embodiments, the reaction chambers are arrayed along the standard spiral track of a compact disc. In some embodiments, the reaction chambers are arrayed in circular tracks concentric about a central axis. In some embodiments, the reaction chambers are arrayed along the standard spiral track of a DVD disk. In some embodiments, the reaction chambers are arrayed along the standard spiral track of a compact disc. In some embodiments, the reaction chambers are liposomes arrayed into a semi-structured three-dimensional cluster by streptavidin tethers that link neighboring liposomes. In some embodiments, the reaction chambers are vesosomes arrayed into a semi-structured three-dimensional cluster by streptavidin tethers that link neighboring vesosomes.

In some embodiments, the reaction chambers are contained within a living cell. In some embodiments, the reaction chambers are inserted into the living cell using a pipette. In some embodiments, the reaction chambers are inserted into the living cell via lipofection. In some embodiments, the living cell is an artificial cell. In some embodiments, the reaction chambers are inserted into an artificial cell at the moment of the formation of it cell membrane by the hydration of a lipid film by inclusion of a solution of the reaction chambers into the hydrating mixture. In some embodiments, the reaction chambers are contained within living cells which are themselves contained into pits or cavities cut into a disk or wafer of sturdy material.

In some embodiments, primer strands of polymer are contained within the reaction chambers. In some embodiments, the primer strands are, but are not limited to being, genetic material, polypeptides, or hydrocarbon chains. In some embodiments, the primers are genetic material. In some embodiments, the primers are oligonucleotides. In some embodiments, the primer strands are DNA. In some embodiments, the primer strands are DNA sticky-end fragments. In some embodiments, the primer strands are single-stranded DNA.

In some embodiments, primer strands of polymer are contained within the reaction chambers. In some embodiments, the primer strands are, but are not limited to being, genetic material, polypeptides, or hydrocarbon chains. In some embodiments, the primers are genetic material. In some embodiments, the primers are oligonucleotides. In some embodiments, the primer strands are DNA. In some embodiments, the primer strands are DNA sticky-end fragments. In some embodiments, the primer strands are single-stranded DNA. In some embodiments, the primers are a self-assembled monolayer (SAM) of an oligonucleotide thiol (5′SH—(CH₂)₆-T₂₅.

In some embodiments, the floors or the walls of the reaction chamber are coated with bound primers. In some embodiments, the dye or phase change layer, of present, is coated with bound primers. In some embodiments, the reflective metal layer, if present, is coated with bound primers. In some embodiments, the primers float freely in the reaction chamber.

### EXAMPLES

The following Examples provide exemplary, non-limiting embodiments of the presently disclosed subject matter. Certain aspects of the following Examples are disclosed in terms of techniques and procedures found or contemplated by the present inventors to work well in the practice of the embodiments. In light of the present disclosure and the general level of skill in the art, those of skill will appreciate that the following Examples are intended to be exemplary only and that numerous changes, modifications, and alterations can be employed without departing from the scope of the presently claimed subject matter.

### Example 1

### Chemical Recording with Thermosensitive Liposomes and DNA Sticky-End Fragments

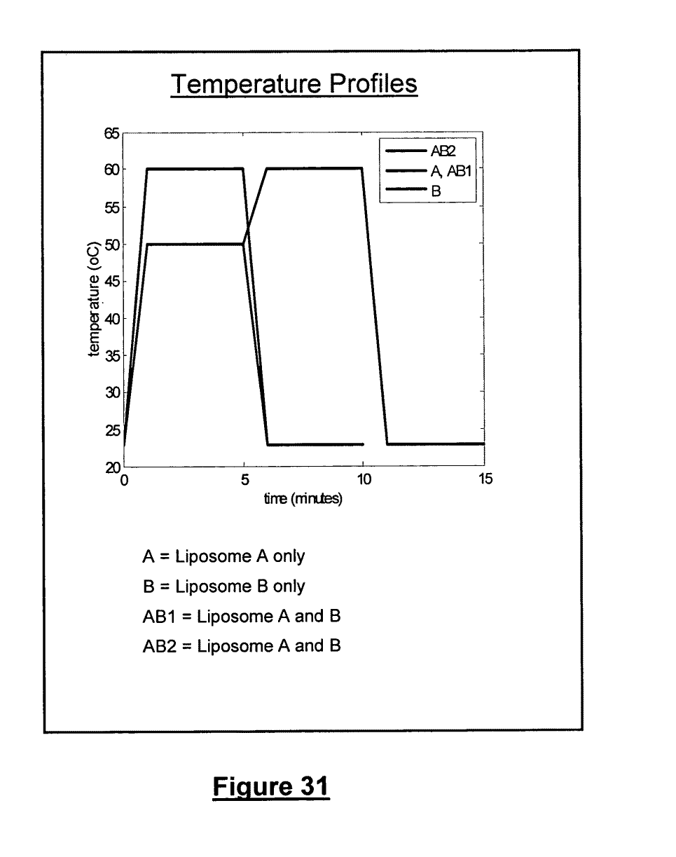

FIGS. 31, 32A-32D, and 33 illustrate an embodiment of the presently disclosed subject matter which was implemented. This embodiment is a composition comprising a plurality of reservoirs, in this particular instance thermosensitive liposomes, and one or more reaction chambers, in this particular instance a test tube, with the reservoirs encapsulating one or more chemical units, in this case DNA sticky-end fragments, with the reservoirs designed to release one or more of the chemical units contained therein if and only if the reservoirs experience an environmental state variable, in this case temperature, above a threshold value, in this case 50° C. or 60° C.

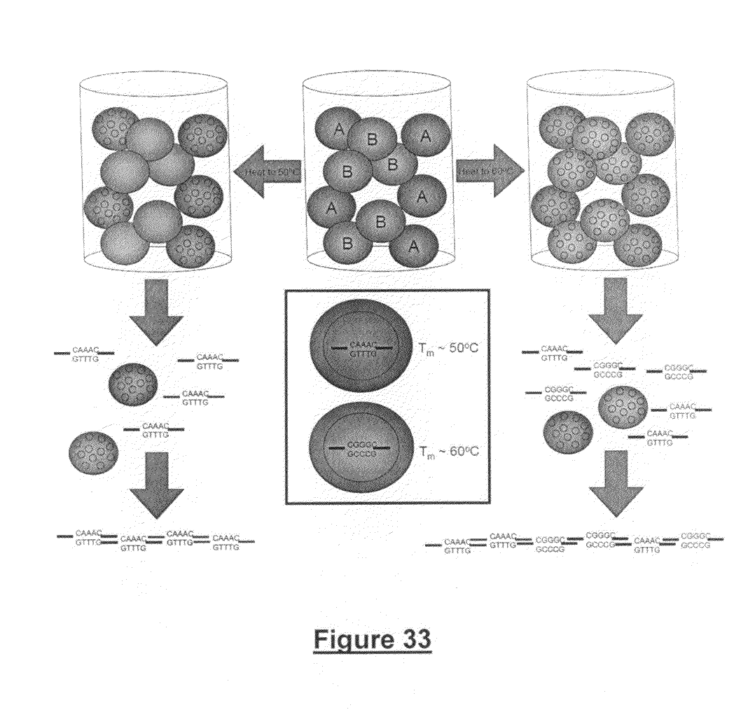

The overall experimental procedure is depicted in FIG. 33. Two varieties of thermosensitive liposomes were used. Liposome A was composed of pure DPPC and generated using the Thin-Film and Extrusion method previously described. Liposome B was composed of pure DSPC. Each liposome variety had a unique transition temperature due to its composition. Liposome A released at approximately 50° C. and Liposome B release at approximately 60° C. The liposomes were passively loaded with a unique DNA sticky end fragment. Liposome A's DNA sticky end fragments had a nucleotide sequence in the overlap region of CAAAG complemented by GTTTG and a sequence ratio of log_{(CG/AT)}=−0.041. Liposome B's DNA sticky end fragments had a nucleotide sequence with an overlap region of CGGGC complemented by GCCCG and sequence ratio of 0.212.

Four temperature profiles were used (see FIG. 31) in order to test the binding of Liposome A and B alone and in combination. In combination, testing was completed to ensure that Liposome B did not release below its transition temperature. In FIG. 31, A denotes Liposome A only; B denotes Liposome B only; and AB1 and AB2 denote both Liposomes A and B.

The ligase was added at one of two stages. Some experiments were run with the ligase and necessary reagents present in solution and 5 μl samples were removed at the appointed times, purified, and sequenced. Other experiments were run without ligase present in solution. In these experiments, ligase was added to the samples taken, which were subsequently purified and sequenced. Multiple samples were taken at each of the appointed time intervals.

Data analysis consisted of taking the sequence data and using a window of varying size and location to determine the logarithm of the ratio of C+G to A+T.

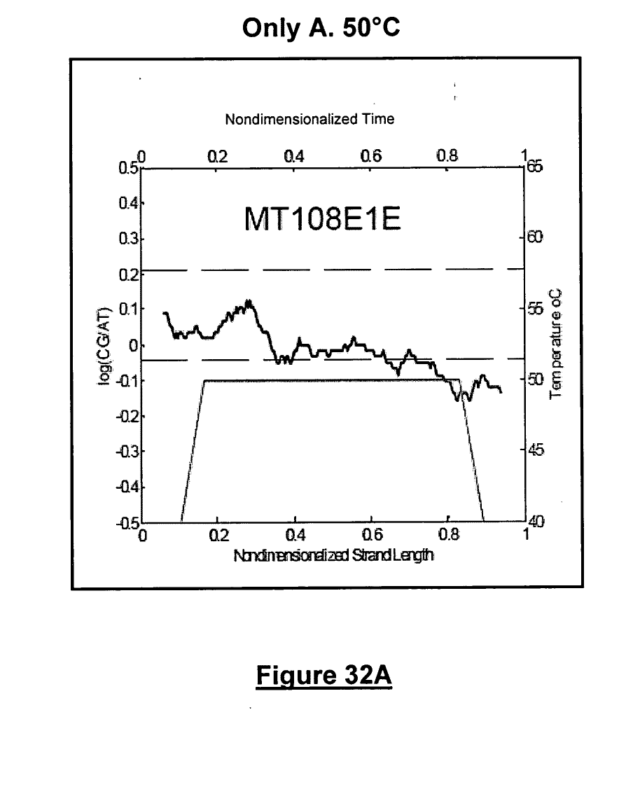

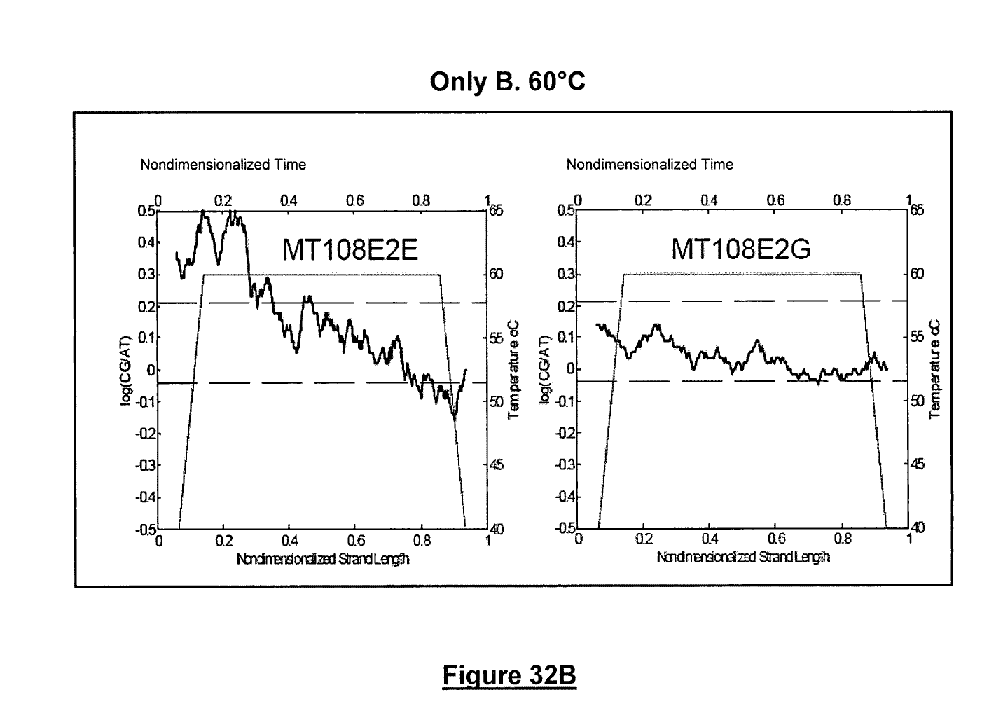

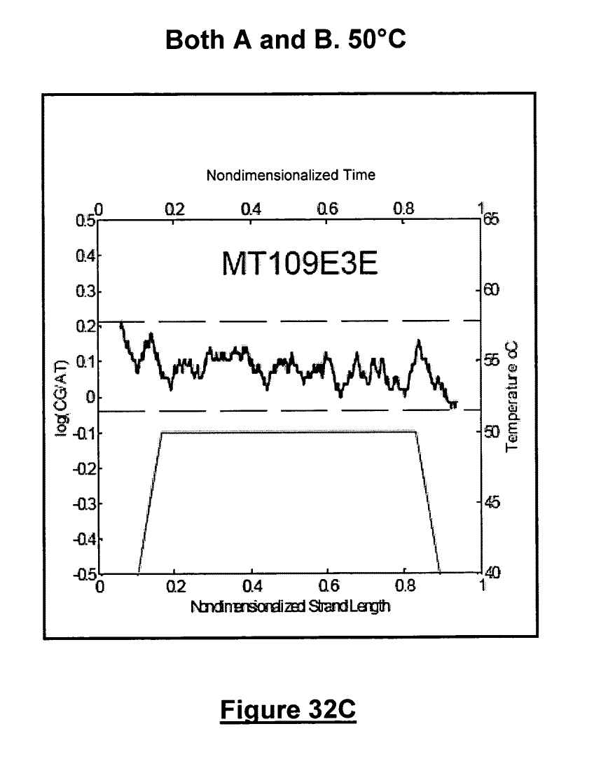

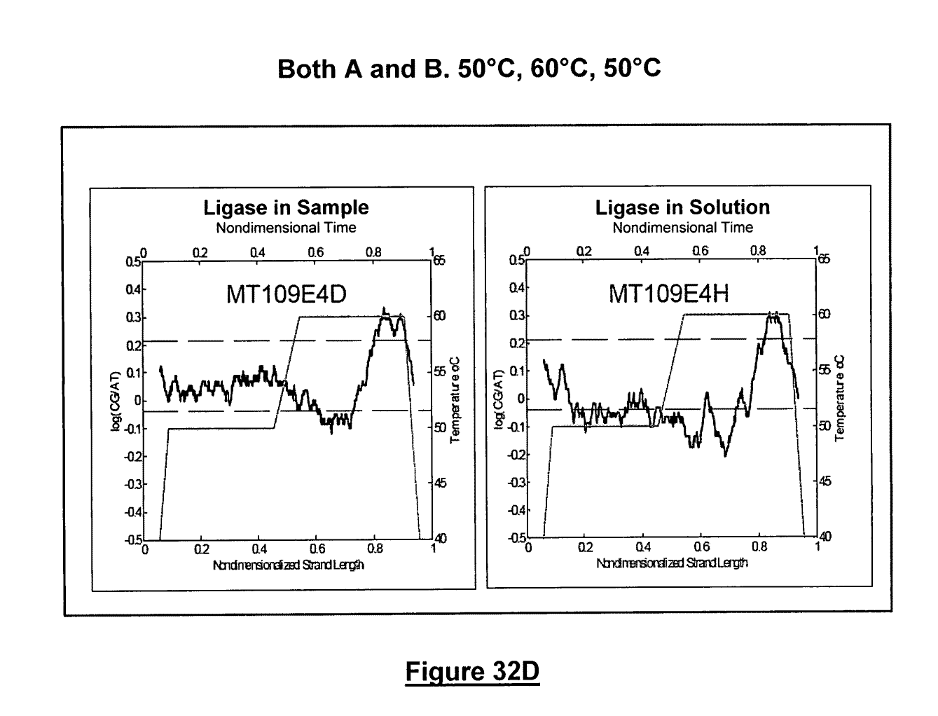

The results from experiments testing Liposome A and B separately at their respective transition temperatures are shown in FIG. 32A. The axes of the figures are clockwise from bottom: Nondimensionalized Strand Length, log_{(CG/AT)}, Nondimensionalized Time, and Temperature (° C.). The blue solid line denotes the log_{(CG/AT)} with respect to time. The red dashed lines represent the respective ratios of the sequences stored in Liposome A and B. The green dotted line represents the temperature profile for the specific experiment.

In these experiments, Liposome A was heated to 50° C. for 5 minutes and then cooled to room temperature immediately by placing the sample on ice. The resulting data strand has a log_{(CG/AT)} value that hovers close to the value of Liposome A of −0.041. Similarly, Liposome B was heated to 60° C. for 5 minutes and then cooled. The data fell into two categories. The data shown in the left of FIG. 32B starts high and decreases throughout the time period while the data shown in the right of the figure keeps a relatively constant value close to the value of Liposome B of 0.212.

The results from the experiments testing the combination of Liposome A and B with two different temperature profiles are shown in FIGS. 32C and 32D. FIG. 32C shows data from experiments testing Liposome A and B at the lower transition temperature of A. FIG. 32D shows data from experiments testing Liposome A and B at both transition temperatures, along with two methods of adding ligase to the experiment. The axes for the figures are, clockwise from bottom: Nondimensionalized Strand Length, log_{(CG/AT)}, Nondimensionalized Time, and Temperature (° C.). The blue solid line denotes the log_{(CG/AT)} with respect to time. The red dashed lines represent the respective ratios of the sequences stored in Liposome A and B. The green dotted line represents the temperature profile for the specific experiment.

In these experiments, both liposomes were present in solution. The data shown in FIG. 32C are from a temperature profile where the temperature was held at 50° C. to allow Liposome A to release but not Liposome B. The resulting data strand appeared to support this design. The data shown in FIG. 32D are from experiments where the temperature profile started at 50° C. and was then increased to 60° C. The data on the left of the figure are from an experiment where the ligase was added to the sample after the sample was taken from the solution. The data on the right is from an experiment where the ligase was present in solution throughout the duration of the experiment. The results from these experiments are particularly interesting. Both share similar data profiles and start as a relatively constant lower ratio value and then increase to the upper ratio value after the temperature was increased.

### Example 2

### Chemical Recording with Thermosensitive Liposomes and Single Strand DNA

In these experiments, a single strand DNA concept was implemented in order to avoid premature binding of the DNA within the liposomes prior to release. In this concept, shown in FIG. 34, each variety of thermosensitive liposome encapsulates a unique 20 to 24 nucleotide long single strand of DNA. The length of the oligonucleotides was balanced between efforts to make it as short as possible while also ensuring that the melting temperature of the strand would be high enough to withstand the elevated liposome transition temperatures. This was done primarily through the GC content. The sequence design also had to ensure that there were no internal structures that would prevent the sequences from properly binding with each other.

In the surrounding solution, in addition to ligase and cofactors, the complement of each of these DNA strands is present, as well as the linkage complement. The linkage complement takes the last half of one DNA strand complement and the first half of another DNA strand complement. As depicted in FIG. 34, solid blue and red bars denote the unique DNA strands (chemical recording unit) and hashed blue and red bars denote the complements to the solid blue and red bars, respectively. In addition to the exact complement of red and blue, linkage complements must also be available. Thus, if a “red” DNA strand and a “blue” DNA strand are released, a complement link must be present in order to record the order. Upon encountering the threshold temperature the unique DNA strands encapsulated within the liposomes (circles) are released, whereby the DNA strands bind with the corresponding complement to form a single strand of DNA (inset), wherein the position and number of each DNA strand (chemical recording unit) in the single strand of DNA is indicative of a reading of the environmental state variable, e.g. temperature, at a given point in time.

Also present in solution is a start sequence. The start sequence is either 20 or 22 nucleotides long. At one end of the start is a double strand stretch of 10 bases that is the same across all the start sequences. The remainder of the start sequence is half of the complement of one of the DNA strand sequences. The start sequence can be selected to be the complement to any of the DNA strands with the original DNA present in solution.

This DNA scheme uses T4 DNA ligase present in solution. It is also possible to add the ligase to samples removed from the solution during experiments.

Experiments were first preformed using DNA and ligase to confirm binding and determine optimal experimental conditions and then were conducted to include liposomes with the DNA and ligase. Experiments that focused on using just DNA strands involved varying the concentration of the DNA, primer, and ligase. The solutions were incubated at the recommended temperature for the ligase and then heated to deactivate the ligase. Then, the solutions were filtered using SEPHADEX® and either prepared for sequencing or run on a 10% polyacrylamide gel. SEPHADEX® filtering is accomplished by adding 1 mL of hydrated SEPHADEX®-50 to a spin column that is placed inside of a 1.5 mL centrifuge tube. It is briefly centrifuged to remove the extra solvent and then centrifuged at 13,000 g for 2-3 minutes to remove the remaining extra solvent. The sample is added down the center of the SEPHADEX® column and centrifuged at 13,000 g for another 2-3 minutes. To prepare for sequencing, known tails must be added to the DNA in order to be able to add successful sequencing primers. Tails are accomplished either through added a single nucleotide with TdTase or through the use of a designed “end cap” with T4 DNA ligase. After the tails are added, the ligase is again deactivated and the solution is filtered with SEPHADEX® to remove extraneous tails.

The preparation of liposomes involved the drying of a lipid film, hydration with the desired encapsulation solution, preparation of SEPHADEX® columns, the extrusion of the liposomes to achieve a homogenous distribution, and the filtering of the solution through the SEPHADEX® columns to remove any extraneous DNA from the external solution. Experiments that involved liposomes, ligase, and DNA were heated cycled through various temperature profiles which had built-in incubation periods. The ligase was deactivated and the solution was filtered using SEPHADEX® columns and was either prepared for sequencing or run on a 10% polyacrylamide gel.

In order to confirm binding of the selected DNA and ligase, several experimental trials were undertaken. The combination of the single strand DNA with T4 DNA ligase was also completed. In each sample, 1 μL of a 150 μM solution of “start” was added. Half the samples had “Start 4” and the other half had “Start 5”. The samples that had Start 4 contained either 150 μM of liposome DNA “4” or 150 μM of both liposome DNA “4” and “5”. The samples that had Start contained either 150 μM liposome DNA “5” or 150 μM of both liposome DNA “4” and “5”. All the samples contained 150 μM of each of the complements to the liposome DNA. The experiment sample set-ups are summarized in Table 10, including the amounts of single strand DNA used in each sample.

TABLE 10
Experimental set-up for ligation experiments.

| | |
|---|---|
| Bucket DNA | Liposome DNA |

| | Name | μL | Name | μL |
|---|---|---|---|---|
| A | Start 4 | 1 | 4 | 1.8 |
| | 4 bar | 1.8 | | |
| | 5 bar | 1.8 | | |
| | 4 bar/5 bar | 1.8 | | |
| | 5 bar/4 bar | 1.8 | | |
| B | Start 5 | 1 | 5 | 1.8 |
| | 4 bar | 1.8 | | |
| | 5 bar | 1.8 | | |
| | 4 bar/5 bar | 1.8 | | |
| | 5 bar/4 bar | 1.8 | | |
| C | Start 4 | 1 | 4 | 1.5 |
| | 4 bar | 1.5 | 5 | 1.5 |
| | 5 bar | 1.5 | | |
| | 4 bar/5 bar | 1.5 | | |
| | 5 bar/4 bar | 1.5 | | |
| D | Start 5 | 1 | 4 | 1.5 |
| | 4 bar | 1.5 | 5 | 1.5 |
| | 5 bar | 1.5 | | |
| | 4 bar/5 bar | 1.5 | | |
| | 5 bar/4 bar | 1.5 | | |

The T4 DNA ligase used were from Promega (Promega Corp., Madison, Wis., United States of America) and USB (USB Corp., Cleveland, Ohio, United States of America)—one of each sample for each ligase. The Bucket DNA and Liposome DNA were mixed in solution and the ligase added. As per manufacturer's instructions, the solutions were incubated for 10 minutes at room temperature and then the ligases were heat inactivated at 65° C. for 10 minutes. The samples were then filtered using SEPHADEX®-50 spin columns to remove any DNA sequences that were not bound from the solution. The concentrations of the solutions were measured on a Nanodrop spectrophotometer (Thermo Fisher Scientific, Inc., Waltham, Mass., United States of America) and the results are summarized in 11.

TABLE 11
Concentration data from samples obtained from Nanodrop.

| Sample | ng/μL | 260/280 | 260/230 |
|---|---|---|---|
| A (Promega) | 20.5 | 2.00 | 1.67 |
| B (Promega) | 97.9 | 2.04 | 2.04 |
| C (Promega) | 88.1 | 2.02 | 1.97 |
| D (Promega) | 147.9 | 2.00 | 1.99 |
| A (USB) | 2.5 | 2.09 | 0.84 |
| B (USB) | 147.8 | 2.01 | 2.12 |
| C (USB) | 168.8 | 1.94 | 2.10 |
| D (USB) | 32.6 | 1.84 | 1.90 |

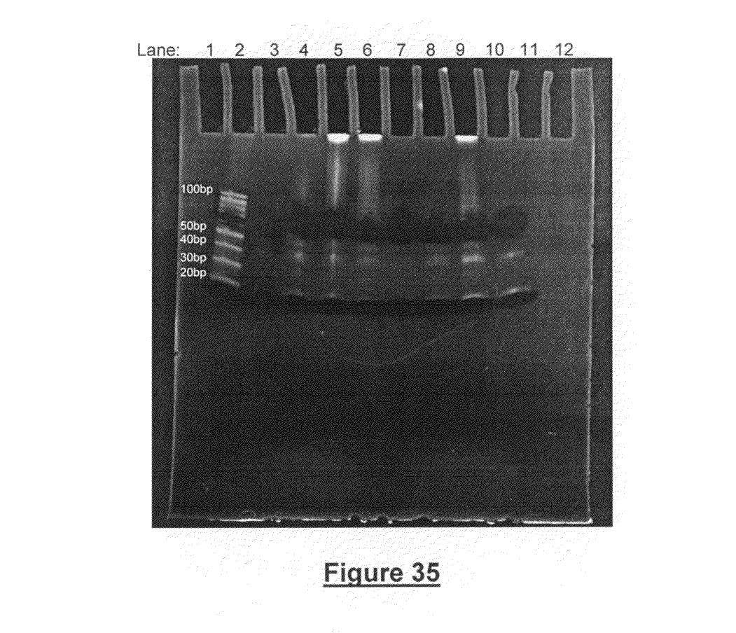

The samples were then run on a 10% polyacrylamide gel and the results are shown in FIG. 35. In FIG. 35, Lane 2 is a 20/100 DNA ladder (DNA fragments of 20, 30, 40, 50, 60, 70, 80, 90 and 100 bp); Lanes 3-6 are samples A-D ligated with the Promega kit; Lanes 7-10 are samples A-D ligated with the USB kit. All the lanes show a band at 20 bp which would indicate that the unbound sequences were not removed with the SEPHADEX® column. The bands at 30 bp would indicate that the liposome DNA bound with a start. Bands at 40 bp indicate that the liposome DNA bound with one of the complement sequences. Bands at 50 bp indicate that the liposome DNA bound with a start and a complement sequence. The smears above 50 bp indicate that the liposome DNA and complement sequences bound into longer strands. Thus, as expected, Lanes 5, 6, and 9, which contain either Samples C and D, demonstrate the generation of the longest sequences as evidenced by the smears above 50 bp.

These experiments were also conducted to prove and quantify the amount of DNA that is encapsulated in the liposomes after hydration. A 50 mg DPPC film was dried and subsequently hydrated with 2 mL of 150 μM single strand DNA solution or 3.45E-7 mol. After hydration, 1.78 mL were recovered and distributed among three samples: 0.20 mL went into sample (A), 0.38 mL went into sample (B) and 1.2 mL went into sample (C). The supernatants of samples A and B were measured to be 1095.9 ng/μl or 3.6E-8 mol and 1046 ng/μl or 6.55E-8 mol respectively. Sample C went on to be extruded using a 1 μm filter. After extrusion, 0.8 mL of solution was recovered and the supernatant was measured to be 656 ng/μl or 2.13E-8 mol. The extruded solution was distributed among two samples: 0.6 mL went into Sample (D) and 0.2 mL went into Sample (E). Sample (D) was filtered using a SEPHADEX® column and Samples (B) and (E) were filtered using a settling method in which the liposomes were repeatedly allowed to settle at the bottom of the tube, the supernatant was carefully removed, and an equal volume of DNase/RNase free water was added back. Once the solution surrounding the liposomes decreased to approximately 6 ng/μl, the samples were heated to 41° C. for 20 minutes. Afterwards, the supernatant concentration was measured at 193.1 ng/μl for Sample (B) and 158.3 ng/μl for Sample (E). This significant increase indicates that the liposomes did in fact encapsulate DNA, which was subsequently released upon heating to the transition temperature.

It will be understood that various details of the presently disclosed subject matter may be changed without departing from the scope of the presently disclosed subject matter. Furthermore, the foregoing description is for the purpose of illustration only, and not for the purpose of limitation.

### REFERENCES

All references cited herein, including but not limited to patents, patent applications and publications thereof, scientific publications, database entries, and references available on the World Wide Web, are incorporated herein by reference to the extent that they supplement, explain, provide a background for, or teach methodology, techniques, and/or compositions employed herein.
Adleman (1994) Molecular Computation of Solutions to Combinatorial Problems, Science 266:1021-1024.
Allen et al., (1995) A new strategy for attachment of antibodies to sterically stabilized liposomes resulting in efficient targeting to cancer cells. Biochimica et Biophysica Acta 1237:99-108.
Andresen et al., (2004) Enzymatic Release of Antitumor Ether Lipids by Specific Phospholipase A₂Activation of Liposome-Forming Prodrugs. J Med Chem 47:1694-1703.
Arap et al. (1998) Cancer treatment by targeted drug delivery to tumor vasculature in a mouse model. Science 279:377-380.
Aronov et al., (2003) Folate-Targeted PEG as a Potential Carrier for Carboplatin Analogs. Synthesis and in Vitro Studies. Bioconjugate Chem, 14:563-574.
Austin et al., (2004) Fabrication of 5 nm linewidth and 14 nm pitch features by nanoimprint lithography, Applied Physics Letters 84:5299-5301.
Bachtold et al. (2001) Logic Circuits with Carbon Nanotube Transistors, Science 294:1317-1320.
Bassett et al., (1986) Use of temperature-sensitive liposomes in the selective delivery of methotrexate and cis-platinum analogues to murine bladder tumor. J Urol 135:612-615.
Cavanihac (2002) Statoliths, in Micscape Magazine, December 2002.
Cherepanov & de Vries (2003) Kinetics and thermodynamics of nick sealing by T4 DNA ligase, European Journal of Biochemistry 270:4315-4325.
Chiu et al. (2005) Encapsulation of doxorubicin into thermosensitive liposomes via complexation with the transition metal manganese. Journal of Controlled Release 104:271-288.
Chou et al. (1996) Nanoimprint lithography, Journal of Vacuum Science & Technology B 14:4129-4133.
Chow et al. (2005) Enzymatic Fabrication of DNA Nanostructures: Extension of a Self-assembled Oligonucleotide Monolayer on Gold Arrays. J Am Chem Soc 127:14122-14123.
Conlisk et al. (2002) Mass Transfer and Flow in Electrically Charged Micro- and Nanochannels, Analytical Chemistry 74:2139-2150.
“Critical point drying incorporating Emitech K850” (1999) Emitech Ltd. de Boer et al. (2000) Micromachining of buried micro channels in silicon, Journal of Microelectromechanical Systems 9:94-103.
“Deep reactive ion etching”, Wikipedia, The Free Encyclopedia, available on the World Wide Web.
Dos Santos et al. (2002) Improved retention of idarubicin after intravenous injection obtained for cholesterol-free liposomes. Biochim Biophys Acta 1561:188-201.
Drummond et al. (2000) Current status of pH-sensitive liposomes in drug delivery. Progress in Lipid Research 39:409-460.
Ferretti & Sgaramella (1981) Specific and reversible inhibition of the blunt end joining activity of the T4 DNA ligase, Nucleic Acids Research 9:3695-3705.
Fowler & Suo (2006) Biochemical, Structural, and Physiological Characterization of Terminal Deoxynucleotidyl Transferase. Chem. Rev 106:2092-2110.
Gaber et al. (1995) Thermosensitive Sterically Stabilized Liposomes: Formulation and in Vitro Studies on Mechanism of Doxorubicin Release by Bovine Serum and Human Plasma. Pharmaceutical Research 12:1407-1416.
Gaber et al. (1996) Thermosensitive Liposomes: Extravasation and Release of Contents in Tumor Microvascular Networks. Int J Radiation Oncology Biol Phys 36:1177-1187.
Gates et al. (2004) Unconventional nanofabrication, Annual Review of Materials Research 34:339-372.
Gregoriadis & Davis (1979) Stability of liposomes in vivo and in vitro is promoted by their cholesterol content and the presence of blood cells. Biochem Biophys Res Commun 89:1287-1293.
Hayashi et al. (1998) Temperature-dependent associating property of liposomes modified with a thermosensitive polymer. Bioconjugate Chem 9:382-389.
Hood et al. (2002) Tumor regression by targeted gene delivery to the neovasculature. Science 296:2404-2407.
Ickenstein et al. (2003) Disc formation in cholesterol-free liposomes during phase transition. Biochim Biophys Acta 1614:135-138.
Iga et al. (1993) Rates of systemic degradation and reticuloendothelial system (RES) uptake of thermosensitive liposome encapsulating cisplatin in rats. Pharmaceutical Research 10:1332-1337.
Itani et al. (2002) Effect of high numerical aperture lens on lithographic performance in 157 nm lithography, Journal of Vacuum Science & Technology B 20:2562-2566.
Kato et al. (1967) Deoxynucleotide-polymerizing Enzymes of Calf Thymus Gland. II. Properties Of The Terminal Deoxynucleotidyltransferase. J Biol Chem 242:2780-2789.
Kirby et al. (1980) Effect of the cholesterol content of small unilamellar liposomes on their stability in vivo and in vitro. Biochem J 186:591-598.
Kong & Dewhirst (1999) Hyperthermia and liposomes. International Journal of Hyperthermia 15:345-70.
Lasic (1997) Liposomes with in liposomes. Nature 387:26-27.
Lasic & Needham (1995) The “Stealth” Liposome: A Prototypical Biomaterial. Chem Rev 95:2601-2628.
Lehman (1974) DNA Ligase: Structure, Mechanism, and Function. Science 186:790-797.
Lindner et al. (2004) Novel temperature-sensitive liposomes with prolonged circulation time. Clin Cancer Res 10:2168-2178.
Lu & Low (2002) Folate-mediated delivery of macromolecular anticancer therapeutic agents. Advanced Drug Delivery Reviews 54:675-693.
Lu & Low (2003) Immunotherapy of folate receptor-expressing tumors: review of recent advances and future prospects. Journal of Controlled Release 91:17-29.
Lukacs et al. (2000) Size-dependent DNA mobility in cytoplasm and nucleus, Journal of Biological Chemistry 275:1625-1629.
Luo & Chakraborty (2005) A novel approach to fabricate metallic nano-cantilevers, Proceedings of SPIE 5763:175-183.
Madou (2002) Fundamentals of Microfabrication, Second ed., CRC Press, Boca Raton, Fla.
Magin & Niesman (1984) Temperature-dependent drug release from large unilamellar liposomes. Cancer Drug Deliv 1:109-117.
“Material Index” (2006) MEMS and Nanotechnology Clearinghouse.
Mayer et al. (1986) Vesicles of variable sizes produced by a rapid extrusion procedure. Biochim Biophys Acta 858:161-168.
Merlin (1991) Encapsulation of doxorubicin in thermosensitive small unilamellar vesicle liposomes. Eur J Cancer 27:1026-1030.
Mijatovic et al. (2005) Technologies for nanofludic systems: top-down vs. bottom-up—a review, Lab on a Chip 5:492-500.
Mills & Needham (2004) The Materials Engineering of Temperature-Sensitive Liposomes. Methods in Enzymology 387:82-113.
Mills & Needham (2005) Lysolipid incorporation in dipalmitoylphosphatidylcholine bilayer membranes enhance the ion permeability and drug release rates at the membrane phase transition. Biochimica et Biophysica Acta 1716:77-96.
Needham & Dewhirst (2001) The development and testing of a new temperature-sensitive drug delivery system for the treatment of solid tumors. Advanced Drug Delivery Reviews 53:285-305.
Needham et al. (2000) A New Temperature-sensitive Liposome for Use with Mild Hyperthermia: Characterization and Testing in a Human Tumor Xenograft Model. Cancer Res 60:1197-1201.
Ness et al. (2004) Buoyancy-Driven Polymerase Chain Reaction (PCR) Devices, presented at Transport Phenomena in Micro and Nano Devices, Kona Coast, Hi.
Nilsson et al. (2003) Fabrication and mechanical characterization of ultrashort nanocantilevers, Applied Physics Letters 83:990-992.
Olson et al. (1979) Preparation of liposomes of defined size distribution by extrusion through polycarbonate membranes. Biochim BiophysActa 557:9-23.
Ono et al. (2002) Reconsideration of drug release from temperature-sensitive liposomes. Biol Pharm Bull 25:97-101.
Papahadjopoulos et al. (1973) Phase transitions in phospholipid vesicles. Fluorescence polarization and permeability measurements concerning the effect of temperature and cholesterol. Biochimica et Biophysica Acta 311:330-348.
Park et al. (2001) Tumor targeting using anti-her2 immunoliposomes. Journal of Controlled Release 74:95-113.
Park et al. (2002) Anti-HER2 Immunoliposomes: Enhanced EfficacyAftributable to Targeted Delivery. Clin Cancer Res 8:1172-1181.
Park et al. (2006) Encapsulated submillimeter piezoresistive accelerometers, Journal of Microelectromechanical Systems 15:507-514.
Perry & Kandlikar (2005) Review of fabrication of nanochannels for single phase liquid flow, presented at 3rd International Conference on Microchannels and Minichannels, Toronto, Ontario, Canada.
Ponce (2007) The development and utilization of magnetic resonance-imageable temperature sensitive liposomes. Duke University: Durham, N.C.
Ponce et al. (2007) Hyperthermia mediated liposomal drug delivery. Int J Hyperthennia 22:205-213.
Rossi et al. (1997) Functional characterization of the T4 DNA ligase: a new insight into the mechanism of action. Nucleic Acids Research 25:2106-2113.
Sandström et al. (2005) Effects of lipid segregation and lysolipid dissociation on drug release from thermosensitive liposomes. J Control Release 107:131-42.
Sapra & Allen (2003) Ligand-targeted liposomal anticancer drugs. Progress in Lipid Research 42:439-462.
Sgaramella et al. (1970) Studies on Polynucleotides, C.* A Novel Joining Reaction Catalyzed by the T4-Polynucleotide Ligase. Proc Natl Acad Sci USA 67:1468-1475.
Shum et al. (2001) Phototriggering of liposomal drug delivery systems. Advanced Drug Delivery Reviews 55:273-284.
Sievers et al. (1989) Statoliths and microfilaments in plant cells, Planta 179:275-278.
Sugino et al. (1977) Interaction of Bacteriophage T4 RNA and DNA Ligases in Joining of Duplex DNA at Base-paired Ends. Journal of Biological Chemistry 252:3987-3994.
Szoka & Papahadjopoulos (1978) Procedure for preparation of liposomes with large internal aqueous space and high capture by reverse-phase evaporation. Proc Natl Acad Sci USA 75:419-44198.
Szoka Jr. et al. (1980) Comparative Properties and Methods of Preparation of Lipid Vesicles (Liposomes). Ann Rev Biophys Bioeng 9:467-508.
Tanner & Protz (2007a) The feasibility of a nano-interial measurement unit that uses chemistry to record position. in Fountain (ed.), Chemical and Biological Sensing VIII, Orlando, Fla., SPIE—the International Society for Optical Engineering, Bellingham, Wash., United States of America.
Tanner & Protz (2007b) Design of a nano-IMU with chemical recording. in 10th Joint Conference on Information Sciences, 2007, Salt Lake City. Utah, World Scientific Publishing Co., Inc., Hackensack, N.J., United States of America.
Tans et al. (1998) Room-temperature transistor based on a single carbon nanotube. Nature 393:49-52.
Tas et al. (1996) Stiction in surface micromachining, Journal of Micromechanics and Microengineering 6:385-397.
Tegenfeldt et al., (2004) Micro- and nanofluidics for DNA analysis, Anal Bioanal Chem 278:1678-1692.
Unezaki et al. (1994) Enhanced delivery and antitumor activity of doxorubicin using long-circulating thermosensitive liposomes containing amphipathic polyethylene gylcol in combination with local hyperthermia. Pharmaceutical Research 11:1180-1185.
Unger et al. (1998) Acoustically active lipospheres containing paclitaxel: a new therapeutic ultrasound contrast agent. Investigative Radiology 33:886-892.
U.S. Pat. Nos. 6,221,401; 6,565,889; 6,726,925; and 6,964,778.
Vermehren et al. (1998) Increase in phospholipase A2 activity towards lipopolymer-containing liposomes. Biochimica et Biophysica Acta 1373:27-36.
Vieu (2000) Electron beam lithography: resolution limits and applications, Applied Surface Science 164:111-117.
Walker et al. (1997) Encapsulation of bilayer vesicles by self assembly. Nature 387:61-64.
Weinstein et al. (1980) Treatment of solid L1210 murine tumors with local hyperthermia and temperature-sensitive liposomes containing methotrexate. Cancer Res 40:1388-1395.
Weiss (1996) Cellular Biophysics: Transport, vol. 1, Massachusetts Institute of Technology Press, Cambridge, Mass.
Weiss et al. (1968) Enzymatic Breakage and Joining of Deoxyribonucleic Acid VI. Further purification and properties of polynucleotide ligase from Escherichia Coli infected with bacteriophage T4, Journal of Biological Chemistry 243:4543-4555.
Yatvin et al. (1980a) Induced drug release from lipid vesicles in serum by pH-change. Biophys Struct Mech 6:233-234.
Yatvin et al. (1980b) pH-sensitive liposomes: possible clinical implications. Science 210:1253-1255.

What is claimed is:

1. A method for recording of an environmental state variable, the method comprising generating a polymer comprising an ordered series of chemical units, wherein the position and number of each chemical unit in the polymer is indicative of a reading of the environmental state variable at a given point in time.

2. The method of claim 1, wherein the environmental state variable is selected from the group consisting of position, velocity, acceleration, temperature, pressure, fluorescence, concentration, and pH, intensity of sound, intensity of light or electromagnetic radiation, and strength of magnetic field.

3. The method of claim 1, wherein the chemical units are selected from the group consisting of sugars, amino acids, and nucleotides.

4. The method of claim 3, wherein the chemical units are nucleotides.

5. The method of claim 4, further comprising determining the nucleotide sequence of the polymer.

6. The method of claim 1, wherein the chemical units are present within a plurality of reservoirs, and further wherein:
(a) each member of the plurality of reservoirs is designed to release one or more of the chemical units present therein when the reservoir experiences an environmental state variable that exceeds a minimum threshold; and
(b) each of the one or more chemical units that are released enters a reaction chamber in which the polymer is generated.

7. The method of claim 6, wherein each of the plurality of reservoirs comprises a thermosensitive liposome designed to release one or more chemical units contained therein if the thermosensitive liposome experiences a temperature exceeding a minimum temperature.

8. The method of claim 7, wherein the plurality of reservoirs comprises at least two different classes of thermosensitive liposomes, each class of thermosensitive liposomes having a different threshold above which the thermosensitive liposome releases one or more chemical units contained therein.

9. The method of claim 8, wherein the chemical units present within the thermosensitive liposomes are identical among members of the same class of thermosensitive liposomes but are different among different classes of thermosensitive liposomes.

10. The method of claim 9, wherein each chemical unit comprises a double stranded region and a single stranded overhang, and further wherein the chemical units present within different classes of thermosensitive liposomes differ in the sequence of the double stranded region but not the sequence of the single stranded overhang.

11. The method of claim 9, wherein each chemical unit comprises a single nucleotide, and further wherein the chemical units present within different classes of thermosensitive liposomes differ in the particular type of nucleotide.

12. The method of claim 9, wherein each chemical unit comprises a single DNA strand, and further wherein the chemical units present within different classes of thermosensitive liposomes differ in the sequence of the single DNA strand.

13. The method of claim 12, further comprising a DNA strand complement.

14. The method of claim 12, further comprising a start sequence.

15. The method of claim 6, wherein the reaction chamber comprises an enzyme that polymerizes the chemical units present therein to form the polymer.

16. The method of claim 15, wherein the enzyme is selected from the group consisting of a ligase and a terminal deoxynucleotidyl transferase.

17. The method of claim 16, wherein the enzyme is a ligase and the reaction chamber further comprises all reagents necessary to produce the polymer.

18. The method of claim 6, wherein each of the plurality of reservoirs comprises a photosensitive liposome designed to release one or more chemical units contained therein if the photosensitive liposome experiences an incident light intensity exceeding a minimum threshold.

19. The method of claim 18, wherein the plurality of reservoirs comprises at least two different classes of photosensitive liposomes, each class of photosensitive liposomes having a different threshold above which the photosensitive liposome releases one or more chemical units contained therein.

20. The method of claim 19, wherein the chemical units present within the photosensitive liposomes are identical among members of the same class of photosensitive liposomes but are different among different classes of photosensitive liposomes.

21. The method of claim 20, wherein each chemical unit comprises a double stranded region and a single stranded overhang, and further wherein the chemical units present within different classes of photosensitive liposomes differ in the sequence of the double stranded region but not the sequence of the single stranded overhang.

22. The method of claim 20, wherein each chemical unit comprises a single DNA strand, and further wherein the chemical units present within different classes of photosensitive liposomes differ in the sequence of the single DNA strand.

23. The method of claim 22, further comprising a DNA strand complement.

24. The method of claim 22, further comprising a start sequence.

25. The method of claim 6, wherein each of the plurality of reservoirs comprises a protein or protein complex designed to release one or more chemical units contained therein by undergoing a conformational change in response to a stimulus such as a change in the temperature, pressure, salinity, or pH, of the environment which surround the protein or protein complex or to a binding event with an antigen or other molecule or particle.

26. The method of claim 25, wherein the plurality of reservoirs comprises at least two different classes of protein or protein complex, each class of which is responsive to a different stimulus.

27. The method of claim 26, wherein the chemical units present within the proteins or protein complexes are identical among members of the same class of proteins or protein complexes but are different among different classes of proteins or protein complexes.

28. The method of claim 27, wherein each chemical unit comprises a double stranded region and a single stranded overhang, and further wherein the chemical units present within different classes of proteins or protein complexes differ in the sequence of the double stranded region but not the sequence of the single stranded overhang.

29. The method of claim 6, wherein each of the plurality of reservoirs comprises a porous or perforated shell designed to release one or more chemical units contained therein by undergoing a change in porosity in response to a stimulus such as a change in the temperature, pressure, salinity, or pH, of the environment which surround the protein or protein complex or to a binding event with an antigen or other molecule or particle.

30. The method of claim 29, wherein the porous or perforated shell is made from a plastic or organic polymer.

31. The method of claim 30, wherein the plurality of reservoirs comprises at least two different classes of porous or perforated shell, each class of which is responsive to a different stimulus.

32. The method of claim 31, wherein the chemical units present within the porous or perforated shells are identical among members of the same class of proteins or protein complexes but are different among different classes of porous or perforated shells.

33. The method of claim 32, wherein each chemical unit comprises a double stranded region and a single stranded overhang, and further wherein the chemical units present within different classes of porous or perforated shells differ in the sequence of the double stranded region but not the sequence of the single stranded overhang.

34. The method of claim 31, wherein the porous or perforated shell is made from a ceramic.

35. The method of claim 31, wherein the porous or perforated shell is made from a metal.

36. The method of claim 32, wherein each chemical unit comprises a single DNA strand, and further wherein the chemical units present within different classes of porous or perforated shells differ in the sequence of the single DNA strand.

37. The method of claim 36, further comprising a DNA strand complement.

38. The method of claim 36, further comprising a start sequence.

39. The method of claim 6, wherein the reaction chamber is a liposome.

40. The method of claim 39, wherein the reaction chamber is a liposome contained within a vesosome.

41. The method of claim 6, wherein the reaction chamber is a vesosome.

42. The method of claim 6, wherein sensed information is simultaneously reported it is being chemically recorded.

43. The method of claim 42, wherein the reporting signal is a level of fluorescence.

44. The method of claim 43, wherein a particular fluorophore is associated with DNA binding so that the detectable fluorescence is altered as units are bound and/or to particular sequences so that the detectable fluorescence is altered in the mixing chamber as more chemical units are added.

45. The method of claim 1, wherein the chemical units are selected from the group of small particles, which may or may not be chemically inert.

46. The method of claim 45, wherein the small particles are selected from the group consisting of metal beads, plastic beads, ferromagnetic beads, electrostatically-charged dielectric beads.

47. The method of claim 1, wherein the chemical units are selected from the group consisting of bacteria, archaea, and eukaryotic cells.

48. The method of claim 1, wherein the chemical units are bound to a plurality of surfaces, and further wherein:
(a) each member of the plurality of surfaces is designed to release one or more of the chemical units present thereon when the surface experiences an environmental state variable that exceeds a minimum threshold; and
(b) each of the one or more chemical units that are released enters a reaction chamber in which the polymer is generated.

49. The method of claim 1, wherein the chemical units are initially possessed of one of a plurality of specific conformations, each of which makes the chemical units unavailable for incorporation into a polymer, and further wherein:
(a) one or more of the chemical units which possess a conformation corresponding to each member of the plurality of conformations is designed to take on a new conformation when the unit experiences a change in an environmental state variable that exceeds a minimum threshold; and
(b) each of the one or more chemical units that undergoes a change in conformation adopts a new conformation that makes it available for incorporation into the polymer that is being generated in the reaction chamber.

50. A composition for chemical recording comprising:
(a) a plurality of reservoirs each containing one or more chemical units, wherein each reservoir is characterized by a thermal stability point at or above which one or more of the chemical units present within the reservoir is released from the reservoir;
(b) a reaction chamber in which the one or more chemical units that have been released collect; and
(c) an enzyme and all other reagents necessary for polymerizing the one or more chemical units present in the reaction chamber to form a polymer.

51. The composition of claim 50, wherein the plurality of reservoirs comprises one or more different classes of reservoirs, each class of reservoir having a different threshold above which the reservoir releases one or more chemical units contained therein.

52. The composition of claim 51, wherein the plurality of reservoirs comprises at least two different classes of thermosensitive liposomes, and further wherein each class of thermosensitive liposomes is characterized by a different threshold above which the thermosensitive liposome releases one or more of the chemical units contained therein.

53. The composition of claim 52, wherein the chemical units present within the thermosensitive liposomes are identical among members of the same class of thermosensitive liposomes but are different among different classes of thermosensitive liposomes.

54. The composition of claim 53, wherein each chemical unit comprises a nucleic acid molecule comprising a double stranded region and a single stranded overhang, and further wherein the chemical units present within different classes of thermosensitive liposomes differ in the sequence of the double stranded region but not the sequence of the single stranded overhang.

55. The composition of claim 53, wherein each chemical unit comprises a single DNA strand, and further wherein the chemical units present within different classes of thermosensitive liposomes differ in the sequence of the single DNA strand.

56. The composition of claim 55, further comprising a DNA strand complement.

57. The composition of claim 55, further comprising a start sequence.

58. The composition of claim 50, wherein the reaction chamber comprises an enzyme that polymerizes the chemical units present therein to form the polymer.

59. The composition of claim 58, wherein the enzyme is selected from the group consisting of a ligase and a terminal deoxynucleotidyl transferase.

60. The composition of claim 59, wherein the enzyme is a ligase and the reaction chamber further comprises all reagents necessary to produce the polymer.

61. A composition for chemical recording comprising:
(a) a plurality of photosensitive liposomes each containing one or more chemical units, wherein each photosensitive liposome is characterized by a stability point governed by the intensity of light or electromagnetic radiation incident on the reservoir such that beyond a given threshold for said light intensity one or more of the chemical units present within the reservoir is released from the reservoir;
(b) a reaction chamber in which the one or more chemical units that have been released collect; and
(c) an enzyme and all other reagents necessary for polymerizing the one or more chemical units present in the reaction chamber to form a polymer.

62. The composition of claim 61, wherein the plurality of reservoirs comprises one or more different classes of photosensitive liposomes, each class of reservoir having a different threshold above which the reservoir releases one or more chemical units contained therein.

63. The composition of claim 62, wherein the plurality of reservoirs comprises at least two different classes of photosensitive liposomes, and further wherein each class of photosensitive liposomes is characterized by a different threshold above which the photosensitive liposome releases one or more of the chemical units contained therein.

64. The composition of claim 63, wherein the chemical units present within the photosensitive liposomes are identical among members of the same class of photosensitive liposomes but are different among different classes of photosensitive liposomes.

65. The composition of claim 64, wherein each chemical unit comprises a nucleic acid molecule comprising a double stranded region and a single stranded overhang, and further wherein the chemical units present within different classes of photosensitive liposomes differ in the sequence of the double stranded region but not the sequence of the single stranded overhang.

66. The composition of claim 64, wherein each chemical unit comprises a single DNA strand, and further wherein the chemical units present within different classes of photosensitive liposomes differ in the sequence of the single DNA strand.

67. The composition of claim 66, further comprising a DNA strand complement.

68. The composition of claim 66, further comprising a start sequence.

69. The composition of claim 61, wherein the reaction chamber comprises an enzyme that polymerizes the chemical units present therein to form the polymer.

70. The composition of claim 69, wherein the enzyme is selected from the group consisting of a ligase and a terminal deoxynucleotidyl transferase.

71. The composition of claim 70, wherein the enzyme is a ligase and the reaction chamber further comprises all reagents necessary to produce the polymer.

72. A method for creating a microorganism that is capable of exhibiting genetic memory by recording the time history of one or more environmental state variables into genetic material stored within its cell or cells, the method comprising inserting a chemical sensing and recording mechanism into a microorganism.

73. The method of claim 72, wherein the chemical sensing and recording mechanism comprises one or more chemical units, reservoirs, valves and reaction chambers.

74. The method of claim 73, wherein the chemical units are nucleotides.

75. The method of claim 73, wherein the reservoirs are liposomes.

76. The method of claim 75, wherein the valves are pores in the liposomes.

77. The method of claim 73, wherein the reaction chamber comprises a liposome or vesosome.

78. The method of claim 73, further comprising TdTase or ligase in the reaction chamber.

79. The method of claim 72, wherein the nucleotide sequence of the stored genetic material can be expressed.

80. The method of claim 72, wherein the recorded genetic material is incorporated into the genome of the microorganism and is inheritable by the offspring of the organism.

81. The method of claim 80, wherein the nucleotide sequence of the stored genetic material can be expressed.

82. The method of claim 72, wherein the recording of the time history of one or more environmental state variables into genetic material comprises generating a polymer comprising an ordered series of chemical units, wherein the position and number of each chemical unit in the polymer is indicative of a reading of the environmental state variable at a given point in time.

83. The method of claim 72, wherein the chemical sensing and recording mechanism is contained within a vesosome.

84. The method of claim 83, wherein the vesosome is inserted into the microorganism using a pipette.

85. The method of claim 83, wherein the vesosome is inserted into the microorganism using a lipofection.

86. The method of claim 83, wherein the vesosome is inserted into an artificial organism as part of the process of assembling that microorganism.

87. The method of claim 72, wherein the chemical and sensing recording mechanism is inserted into an artificial microorganism as part of the process of assembling that microorganism.

88. The method of claim 87, wherein the chemical and sensing recording mechanism is inserted into an artificial organism by including it a mixture or solution that is used to hydrate the lipid film that ultimately becomes the lipid bilayer which constitutes the cell membrane of the artificial organism.

89. The method of claim 72, wherein the method for creating a microorganism that is capable of exhibiting genetic memory by recording the time history of one or more environmental state variables into genetic material stored within its cell or cells yields an organism that is possessed of a programmable genome.

90. A composition for implementing an microorganism which exhibits genetic memory wherein the microorganism contains within it:
(a) one or more sensing and chemical recording mechanisms which record the time history of changes to one or more environmental state variables to which the microorganism is exposed into a strand of genetic material in such a way that the nucleotide sequence in the genetic material provides a record of the time series of those environmental variables; and
(b) a chamber containing a mixture of enzymes and other reagents which are able to incorporate the strand of genetic material written by the chemical recording mechanism into the genetic material of the host microorganism by viral or other mechanisms.

91. The composition of claim 90, wherein the chemical sensing and recording mechanisms comprise:
(a) a plurality of reservoirs each containing one or more chemical units, wherein each reservoir is characterized by a thermal stability point at or above which one or more of the chemical units present within the reservoir is released from the reservoir;
(b) a reaction chamber in which the one or more chemical units that have been released collect; and
(c) an enzyme and all other reagents necessary for polymerizing the one or more chemical units present in the reaction chamber to form a polymer.

92. The composition of claim 90, wherein the chemical sensing and recording mechanisms comprise:
(a) a plurality of photosensitive liposomes each containing one or more chemical units, wherein each photosensitive liposome is characterized by a stability point governed by the intensity of light or electromagnetic radiation incident on the reservoir such that beyond a given threshold for said light intensity one or more of the chemical units present within the reservoir is released from the reservoir;
(b) a reaction chamber in which the one or more chemical units that have been released collect; and
(c) an enzyme and all other reagents necessary for polymerizing the one or more chemical units present in the reaction chamber to form a polymer.

93. The composition of claim 90, wherein the chemical sensing and recording mechanisms are contained within one or more vesosomes.

94. The composition of claim 93, wherein the one or more vesosomes are originally inserted into the microorganism using a pipette.

95. The composition of claim 93, wherein the one or more vesosomes are originally inserted into the microorganism using lipofection.
